(19) United States
(12) Patent Application Publication
(10) Pub. No.: US 2026/0347309 A1
(43) Pub. Date: Nov. 5, 2026

(54) SALTS OF 2-S RIMANTADINE AND 2-R RIMANTADINE FOR TREATING CANCER

(71) Applicant: Toragen, Inc., San Diego, CA (US)

(72) Inventors: Richard Lumpkin, San Diego, CA (US); Daniel Walters, San Diego, CA (US)

(21) Appl. No.: 19/103,769

(22) PCT Filed: Aug. 17, 2023
(86) PCT No.: PCT/US2023/072405

§ 371 Date: Feb. 13, 2025

Related U.S. Application Data
(60) Provisional application No. 63/398,826, filed on Aug. 17, 2022.

(51) Int. Cl. C07C 211/34; A61K 31/13; A61K 31/225; A61P 31/20; A61P 35/00

(52) U.S. Cl. CPC C07C 211/34; A61K 31/13; A61K 31/225; A61P 31/20; A61P 35/00

(57) ABSTRACT

Disclosed herein is the use of purified 2-S rimantadine or purified 2-R rimantadine or a pharmaceutically acceptable thereof to treat cancers and precancer lesions, including cancers and precancer lesions associated with papilloma virus in subjects in need of treatment.

### CROSS-REFERENCE TO RELATED APPLICATION

This application claims the benefit of U.S. Provisional Application No. 63/398,826 filed on Aug. 17, 2022, which is hereby incorporated by reference in its entirety.

### INCORPORATION BY REFERENCE

All publications, patents, and patent applications disclosed herein are incorporated by reference to the same extent as if each individual publication, patent, or patent application was specifically and individually indicated to be incorporated by reference. In the event of a conflict between a term disclosed herein and a term in an incorporated reference, the term herein controls.

### FIELD OF THE INVENTION

The present disclosure relates to methods of treating or preventing cancer, including cancers caused by papilloma viruses comprising administering enantiomerically pure salt of 2-S enantiomer of rimantadine.

### BACKGROUND OF THE DISCLOSURE

Genital human papillomavirus (HPV) is the most common sexually transmitted infection in the United States. The Centers for Disease Control and Prevention (CDC) states that 90% of HPV infections cause no symptoms and resolve spontaneously within two years. However, in some cases, an HPV infection persists and results in either warts or precancerous lesions. These lesions, depending on the site affected, increase the risk of cancer of the cervix, vulva, vagina, penis, anus, rectum, and oropharynx. HPV types associated with cervical oncogenicity are classified into 15 “high-risk types” (HPV 16, 18, 31, 33, 35, 39, 45, 51, 52, 56, 58, 59, 68, 73 and 82) and 3 “possibly high-risk types” (HPV 26, 53 and 66). Researchers have also shown an association of HPV 16 and 18 with breast cancer. HPV types (HPV 6, 11, 40, 42, 43, 44, 54, 61, 70, 72 and 81) are classified as “low risk types” and are known to show benign low-grade cervical changes, genital warts and recurrent respiratory papillomatosis. Skin HPV types 5, 8, and 92 are associated with skin cancer.

Rimantadine hydrochloride (α-methyl-1-adamantane-methalamine hydrochloride) is an oral medication sold under the brand name Flumadine® that is used to treat influenza A. Rimantadine inhibits influenza activity by binding to amino acids in the virus M2 transmembrane channel and blocking proton transport across the M2 channel. Flumadine® contains a racemic mixture of rimantadine. One study found evidence that the R-enantiomer binds the M2 channel pore with greater affinity than the S-enantiomer. However, that finding is in conflict with several earlier findings that found no differences between the enantiomers against M2. The absence of a distinction between the enantiomers against M2 was confirmed in later studies. Rimantadine has also been suggested to have some anti-Parkinsonian activity. However, its use for this indication has not been developed or approved.

Flumadine® has gastrointestinal and central nervous system adverse effects including nausea, upset stomach, vomiting, anorexia, dry mouth, abdominal pain, asthenia, nervousness, tiredness, lightheadedness, dizziness, headache, trouble sleeping, difficulty concentrating, confusion and anxiety. Anxiety and insomnia are the most commonly cited toxicities for discontinuation of treatment.

### SUMMARY OF THE DISCLOSURE

The present disclosure, in some aspects, relates to various salts and solid state forms of 2-S-Rimantidine. In some embodiments, the composition comprises a salt comprising 2-S-Rimantidine and fumaric acid. In some embodiments, the salt is an anhydrous salt. In some embodiments, the salt is crystalline. In some embodiments, the salt has an X-ray powder diffraction comprising peaks at about 6.09 2θ. In some embodiments, the salt has an X-ray powder diffraction further comprises peaks at about 14.36 2θ, about 17.56 2θ, about 18.45 2θ, about 18.81 2θ, and/or about 27.17 2θ. In some embodiments, the composition comprises a salt comprising 2-S-Rimantidine and tartaric acid. In some embodiments, the salt is anhydrous. In some embodiments, the salt is crystalline. In some embodiments, the salt has an x-ray powder diffraction pattern comprising a peak at about 7.50 2θ. In some embodiments, the salt has an x-ray powder diffraction pattern further comprising peaks at about 17.64 2θ, about 18.68 2θ, about 15.43 2θ, about 19.37 2θ, and/or about 22.48 2θ. In some embodiments, the composition comprises a salt comprising 2-S-Rimantidine and galactaric acid. In some embodiments, the salt is anhydrous. In some embodiments, the salt is crystalline. In some embodiments, the salt has an x-ray powder diffraction pattern comprising a peak at about 5.71 2θ. In some embodiments, the salt has an x-ray powder diffraction pattern further comprising peaks at about 15.85 2θ, about 16.96 2θ, about 19.76 2θ, and/or about 19.43 2θ. In some embodiments, the salt has an x-ray powder diffraction pattern comprising a peak at 5.83 2θ. In some embodiments, the salt has an x-ray powder diffraction pattern further comprising peaks at about 14.89 2θ, about 16.87 2θ, about 17.62 2θ, about 30.87 2θ, and/or about 19.72 2θ. In some embodiments, the composition comprises a salt comprising 2-S-Rimantidine and benzoic acid. In some embodiments, the salt is anhydrous. In some embodiments, the salt is crystalline. In some embodiments, the salt has an x-ray powder diffraction pattern comprising a peak at 7.85 2θ. In some embodiments, the salt has an x-ray powder diffraction pattern further comprising peaks at about 9.72 2θ, about 11.49 2θ, about 12.29 2θ, about 15.66 2θ, about 19.06 2θ, about 20.38 2θ, and/or about 29.91 2θ. In some embodiments, the composition comprises a salt comprising 2-S-Rimantidine and benzenesulfonic acid salt. In some embodiments, the salt is anhydrous. In some embodiments, the salt is crystalline. In some embodiments, the salt has an x-ray diffraction powder pattern comprising a peak at 6.21 2θ. In some embodiments, the salt has an x-ray diffraction powder pattern further comprising peaks at about 8.02 2θ, about 8.44 2θ, about 14.11 2θ, about 15.39 2θ, about 16.01 2θ, and/or about 19.08 2θ. In some embodiments, the salt has an x-ray diffraction powder pattern comprising a peak at 8.04 2θ. In some embodiments, the salt has an x-ray diffraction powder pattern further comprising peaks at about 8.45 2θ, about 14.13 2θ, about 15.37 2θ, about 16.86 2θ, and/or about 19.10 2θ. In some embodiments, the composition comprises a crystalline form of 2-S-Rimantidine, wherein the XRPD peaks are about 14.36 2θ, about 17.56 2θ, about 18.45 2θ, about 18.81 2θ, and/or about 27.17 2θ. In some embodiments, the composition comprises a composition comprising a salt of 2-S rimantadine. In some embodiments, the composition comprises a fumarate salt, tartrate salt, galactarate salt, benzoate salt, benzenesulfonate salt, or a combination thereof. In some embodiments, the composition comprises a fumarate salt. In some embodiments, the fumarate salt is fumarate salt type A. In some embodiments, the composition is characterized by one or more of: a peak in an x-ray powder diffraction pattern (“XRPD”) diffractogram at about 6.09° 2θ; a peak in an XRPD diffractogram at about 14.36° 2θ; a peak in an XRPD diffractogram at about 17.56° 2θ; a peak in an XRPD diffractogram at about 18.45° 2θ; a peak in an XRPD diffractogram at about 18.81° 2θ; or a peak in an XRPD diffractogram at about 27.17° 2θ. In some embodiments, the salt is a tartrate salt. In some embodiments, tartrate salt is tartrate salt type A. In some embodiments, the composition is characterized by one or more of: a peak in an x-ray powder diffraction pattern (“XRPD”) diffractogram at about 6.09° 2θ; a peak in an XRPD diffractogram at about 7.50° 2θ; a peak in an XRPD diffractogram at about 17.64° 2θ; a peak in an XRPD diffractogram at about 18.68° 2θ; a peak in an XRPD diffractogram at about 15.43° 2θ; a peak in an XRPD diffractogram at about 19.37° 2θ; or a peak in an XRPD diffractogram at about 22.48° 2θ. In some embodiments, the salt is a galactarate salt. In some embodiments, galactarate salt is galactarate salt type A. In some embodiments, the salt is characterized by one or more of: a peak in an XRPD diffractogram at about 5.71° 2θ; a peak in an XRPD diffractogram at about 15.85° 2θ; a peak in an XRPD diffractogram at about 16.96° 2θ; peak in an XRPD diffractogram at about 19.76° 2θ; or a peak in an XRPD diffractogram at about 19.43° 2θ. In some embodiments, the galactarate salt is galactarate salt type B. In some embodiments, the composition is characterized by one or more of: a peak in an XRPD diffractogram at about 5.83° 2θ; a peak in an XRPD diffractogram at about 14.89° 2θ; a peak in an XRPD diffractogram at about 16.87° 2θ; a peak in an XRPD diffractogram at about 17.62° 2θ; a peak in an XRPD diffractogram at about 30.87° 2θ; or a peak in an XRPD diffractogram at about 19.72° 2θ. In some embodiments, the the salt is a benzoate salt. In some embodiments, the benzoate salt is a benzoate salt type A. In some embodiments, the composition is characterized by one or more of: a peak in an XRPD diffractogram at about 7.85° 2θ; a peak in an XRPD diffractogram at about 9.72° 2θ; a peak in an XRPD diffractogram at about 11.49° 2θ; a peak in an XRPD diffractogram at about 12.29° 2θ; a peak in an XRPD diffractogram at about 15.66° 2θ; a peak in an XRPD diffractogram at about 19.06° 2θ; a peak in an XRPD diffractogram at about 20.38° 2θ; or a peak in an XRPD diffractogram at about 29.91° 2θ. In some embodiments, the salt is a benzenesulfonate salt. In some embodiments, the benzenesulfonate salt is a benzenesulfonate salt type A. In some embodiments, the salt is characterized by one or more of: a peak in an XRPD diffractogram at about 6.21° 2θ; a peak in an XRPD diffractogram at about 8.02° 2θ; a peak in an XRPD diffractogram at about 8.44° 2θ; a peak in an XRPD diffractogram at about 14.11° 2θ; a peak in an XRPD diffractogram at about 15.39° 2θ; a peak in an XRPD diffractogram at about 16.01° 2θ; or a peak in an XRPD diffractogram at about 19.08° 2θ. In some embodiments, the benzenesulfonate salt is a benzenesulfonate salt type B. In some embodiments, the salt is characterized by one or more of: a peak in an x-ray powder diffraction pattern (“XRPD”) diffractogram at about; a peak in an XRPD diffractogram at about 8.04° 2θ; a peak in an XRPD diffractogram at about 8.45° 2θ; a peak in an XRPD diffractogram at about 14.13° 2θ; a peak in an XRPD diffractogram at about 15.37° 2θ; a peak in an XRPD diffractogram at about 16.86° 2θ; or a peak in an XRPD diffractogram at about 19.10° 2θ. In some embodiments, the composition is crystalline. In some embodiments, the differential scanning calorimetry is substantially as illustrated in FIG. 11. In some embodiments, the differential scanning calorimetry is substantially as illustrated in FIG. 12. In some embodiments, the differential scanning calorimetry is substantially as illustrated in FIG. 13. In some embodiments, the differential scanning calorimetry is substantially as illustrated in FIG. 14. In some embodiments, the differential scanning calorimetry is substantially as illustrated in FIG. 15. In some embodiments, the differential scanning calorimetry is substantially as illustrated in FIG. 16. In some embodiments, the differential scanning calorimetry is substantially as illustrated in FIG. 17. In some embodiments, the differential scanning calorimetry is substantially as illustrated in FIG. 18. In some embodiments, the differential scanning calorimetry is substantially as illustrated in FIG. 19. In some embodiments, the differential scanning calorimetry is substantially as illustrated in FIG. 20. In some embodiments, wherein the differential scanning calorimetry is substantially as illustrated in FIG. 21. In some embodiments, the composition comprises a salt of 2-S-Rimantidine, characterized by a sample weight loss of about 0.01 w/w % loss in the thermogravimetric analysis between a temperature of 0 °C and 150 °C. In some embodiments, the salt is a tartaric acid salt. In some embodiments, the composition comprises a salt of 2-S-Rimantidine, characterized by a sample weight loss of about 6.72 w/w % loss in the thermogravimetric analysis between a temperature of 0 °C and 150 °C. In some embodiments, the salt is a maleic acid salt. In some embodiments, the composition comprises a salt of 2-S-Rimantidine, characterized by a sample weight loss of about 10.30 w/w % loss in the thermogravimetric analysis between a temperature of 0 °C and 85 °C. In some embodiments, the salt is a hippuric acid salt. In some embodiments, the composition comprises a salt of 2-S-Rimantidine, characterized by a sample weight loss of about 0.15 w/w % in the thermogravimetric analysis between a temperature of 0 °C and 150 °C. In some embodiments, the salt is a galactaric acid salt. In some embodiments, the composition comprises a salt of 2-S-Rimantidine, characterized by a sample weight loss of about 0.00 w/w % in the thermogravimetric analysis between a temperature of 0 °C and 150 °C. In some embodiments, the salt is a galactaric acid salt. In some embodiments, the composition comprises a salt of 2-S-Rimantidine, characterized by a sample weight loss of about 0.26 w/w % in the thermogravimetric analysis between a temperature of 0 °C and 100 °C. In some embodiments, the salt is a benzoic acid salt. In some embodiments, the composition comprises a salt of 2-S-Rimantidine, characterized by a sample weight loss of about 14.77 w/w % in the thermogravimetric analysis between a temperature of 0 °C and 110 °C. In some embodiments, salt is a benzoic acid salt. In some embodiments, the composition comprises a salt of 2-S-Rimantidine, characterized by a sample weight loss of about 1.68 w/w % loss in the thermogravimetric analysis between a temperature of 0 °C and 150 °C. In some embodiments, the salt is a gentisic acid salt. In some embodiments, the composition comprises a salt of 2-S-Rimantidine, characterized by a sample weight loss of about 3.55 w/w % loss in the thermogravimetric analysis between a temperature of 0 °C and 200 °C. In some embodiments, the salt is a toluensulfonic acid salt. In some embodiments, the composition comprises a salt of 2-S-Rimantidine, characterized by a sample weight loss of about 0.02 w/w % loss in the thermogravimetric analysis between a temperature of 0 °C and 200 °C. In some embodiments, the salt is a benzenesulfonic acid salt. In some embodiments, the composition comprises a salt of 2-S-Rimantidine, characterized by a sample weight loss of about 0.09 w/w % loss in the thermogravimetric analysis between a temperature of 0 °C and 200 °C. In some embodiments, the salt is a benzenesulfonic acid salt. In some embodiments, the composition comprises a salt of 2-S-Rimantidine, wherein the salt exhibits a differential scanning calorimetry comprising an endothermic peak at about 221 °C. In some embodiments, the salt is a tartaric acid salt. In some embodiments, the composition comprises a salt of 2-S-Rimantidine, wherein the salt exhibits a differential scanning calorimetry comprising an endothermic peak at about 63 °C, 74 °C, and 121 °C. In some embodiments, the salt is a maleic acid salt. In some embodiments, the composition comprises a salt of 2-S-Rimantidine, wherein the salt exhibits a differential scanning calorimetry comprising an endothermic peak at about 62 °C. In some embodiments, the salt is a hippuric acid salt. In some embodiments, the composition comprises a salt of 2-S-Rimantidine, wherein the salt exhibits a differential scanning calorimetry comprising an endothermic peak at about 193 °C. In some embodiments, the salt is a galactaric acid salt. In some embodiments, the composition comprises a salt of 2-S-Rimantidine, wherein the salt exhibits a differential scanning calorimetry comprising an endothermic peak at about 195 °C. In some embodiments, the salt is a galactaric acid salt. In some embodiments, the composition comprises a salt of 2-S-Rimantidine, wherein the salt exhibits a differential scanning calorimetry comprising an endothermic peak at about 199 °C. In some embodiments, the salt is a benzoic acid salt. In some embodiments, the composition comprises a salt of 2-S-Rimantidine, wherein the salt exhibits a differential scanning calorimetry comprising an endothermic peak at about 198 °C. In some embodiments, the salt is a benzoic acid salt. In some embodiments, the composition comprises a salt of 2-S-Rimantidine, wherein the salt exhibits a differential scanning calorimetry comprising an endothermic peak at about 206 °C. In some embodiments, the salt is a gentisic acid salt. In some embodiments, the composition comprises a salt of 2-S-Rimantidine, wherein the salt exhibits a differential scanning calorimetry comprising an endothermic peak at about 214 °C. In some embodiments, the salt of the previous claim, wherein the salt is a toluenesulfonic acid salt. In some embodiments, the composition comprises a salt of 2-S-Rimantidine, wherein the salt exhibits a differential scanning calorimetry comprising an endothermic peak at about 223 °C. In some embodiments, the salt is a benzenesulfonic acid salt. In some embodiments, the composition comprises a salt of 2-S-Rimantidine, wherein the salt exhibits a differential scanning calorimetry comprising an endothermic peak at about 232 °C. In some embodiments, the salt is a benzenesulfonic acid salt. In some embodiments, the composition comprises a salt of 2-S-Rimantidine, characterized by crystal particle size between 1-50 μm. In some embodiments, the composition comprises a salt of 2-S-Rimantidine, characterized by an XRPD diffractogram as substantially illustrated in FIG. 4. In some embodiments, the composition comprises a salt of 2-S-Rimantidine, characterized by an XRPD diffractogram as substantially illustrated in FIG. 5. In some embodiments, the composition comprises a salt of 2-S-Rimantidine, characterized by an XRPD diffractogram as substantially illustrated in FIG. 6. In some embodiments, the composition comprises a salt of 2-S-Rimantidine, characterized by an XRPD diffractogram as substantially illustrated in FIG. 7. In some embodiments, the composition comprises a salt of 2-S-Rimantidine, characterized by an XRPD diffractogram as substantially illustrated in FIG. 8. In some embodiments, the composition comprises a salt of 2-S-Rimantidine, characterized by an XRPD diffractogram as substantially illustrated in FIG. 9. In some embodiments, the composition comprises a salt of 2-S-Rimantidine, characterized by an XRPD diffractogram as substantially illustrated in FIG. 10. In some embodiments, the salt of 2-S-rimantadine is a tartaric acid salt of 2-S-rimantadine. In some embodiments, the salt of 2-S-rimantadine is a fumaric acid salt of 2-S-rimantadine. In some embodiments, the salt of 2-S-rimantadine is a galactaric acid salt of 2-S-rimantadine. In some embodiments, the salt of 2-S-rimantadine is a benzoic acid salt of 2-S-rimantadine. In some embodiments, the salt of 2-S-rimantadine is a benzenesulfonic acid salt of 2-S-rimantadine. In some embodiments, the method of the present disclosure comprises a method comprising administering to the subject a therapeutically effective amount of the salt or composition of any one of the previous claims. In some embodiments, the cancer is selected from one or more of melanoma, head and neck cancer, lung cancer, colon cancer, breast cancer, esophageal cancer, pancreatic cancer, prostate cancer, cervical cancer, and stomach cancer. In some embodiments, the cancer is a sarcoma, carcinoma, lymphoma, or leukemia. In some embodiments, the carcinoma is a squamous cell carcinoma. In some embodiments, the squamous cell carcinoma is head and neck squamous cell carcinoma. In some embodiments, the cancer is selected from the group consisting of head and neck cancer, breast cancer, and melanoma. In some embodiments, the cancer is an HPV-associated cancer. In some embodiments, the HPV-associated cancer is associated with the alpha genus of HPV. In some embodiments, one or more cancer cells from the subject express a human papilloma virus (HPV) protein. In some embodiments, the HPV protein is E5, HPV protein. In some embodiments, the HPV E5, protein is from one or more HPV subtypes selected from the group consisting of HPV 6, HPV 11, HPV 16, HPV 18, HPV 31, HPV 33, HPV 35, HPV 39, HPV 45, HPV 51, HPV 52, HPV 56, HPV58, HPV 66, and HPV 69. In some embodiments, the HPV protein is E5 from HPV 16 In some embodiments, the HPV protein is E5 from HPV 18. In some embodiments, the method described comprises a method of treating cancer in a subject, the method comprising: detecting in a sample from the subject a cancer cell that expresses a human papilloma virus (HPV) protein; and administering to the subject a therapeutically effective amount of the salt or the composition of anyone of the previous claims. In some embodiments, the cancer is associated with the alpha genus of HPV. In some embodiments, the HPV protein is one or more of an E5, E6, or E7 HPV protein. In some embodiments, the HPV E5, E6, or E7 protein is from one or more HPV subtypes selected from the group consisting of HPV 6, HPV 11, HPV 16, HPV 18, HPV 31, HPV 33, HPV 35, HPV 39, HPV 45, HPV 51, HPV 52, HPV 56, HPV 58, HPV 66, and HPV 69. In some embodiments, the cancer is selected from the group consisting of head and neck cancer, mucosal squamous cell carcinomas, cutaneous squamous cell carcinomas, hepatic cancer, cervical cancer, vaginal cancer, vulvar cancer, penile cancer, and anal cancer. In some embodiments, the method further comprises administering an additional anti-cancer agent. In some embodiments, the additional anti-cancer agent is selected from the group consisting of: carboplatin, cisplatin, gemcitabine, methotrexate, paclitaxel, pemetrexed, lomustine, temozolomide, dacarbazine, and a combination thereof. In some embodiments, the additional anti-cancer agent is an immunotherapy. In some embodiments, the additional anti-cancer agent is an immune checkpoint inhibitor. In some embodiments, the immune checkpoint inhibitor targets one or more of: CTLA-4, PD-1, PD-L1, BTLA, LAG-3, A2AR, TIM-3, B7-H3, VISTA, and IDO. In some embodiments, the immune checkpoint inhibitor is selected form the group consisting of: ipilimumab, nivolumab, pembrolizumab, atezolizumab, avelumab, durvalumab, tremelimumab, cemiplimab, and a combination thereof. In some embodiments, the method further comprises subjecting the subject to radiation therapy, surgery, or a combination thereof. In some embodiments, the subject is a human. In some embodiments, the method described herein comprises a method of treating a precancerous HPV lesion in a subject needing treatment comprising administering a therapeutically effective amount of the salt or composition of 2-S-Rimantidine. In some embodiments, the HPV lesion is associated with the alpha genus of HPV. In some embodiments, the HPV precancerous lesion is a lesion of the cervix, skin, urethra, nasal cavity, paranasal sinus, larynx, tracheobronchial mucosa or oral cavity. In some embodiments, the HPV precancerous lesion expresses one or more HPV proteins selected from one or more of E5, E6, or E7 HPV protein. In some embodiments, the HPV E5, E6, or E7 protein is from one or more HPV subtypes selected from the group consisting of one or more of HPV 6, HPV 11, HPV 16, HPV 18, HPV 31, HPV 33, HPV 35, HPV 39, HPV 45, HPV 51, HPV 52, HPV 56, HPV 58, HPV 66, and HPV 69. In some embodiments, the rimantadine is administered topically, orally, subcutaneously, or parenterally. In some embodiments, the method described herein comprises a method of treating or preventing avian bird flu in poultry comprising administering a therapeutically effective amount of the salt or the composition of anyone of the previous claims. In some embodiments, the avian bird flu is H5N1. In some embodiments, the side effects associated with administration of the salt or composition of 2-S rimantadine are reduced as compared to the side effects associated with racemic rimantadine or enantiomerically pure 2-R rimantadine. In some embodiments, the composition comprises a composition comprising: a salt or composition of 2-S-Rimantidine, and one or more immune checkpoint inhibitors. In some embodiments, the one or more immune checkpoint inhibitors comprises CTLA-4, PD-1, PD-L1, BTLA, LAG-3, A2AR, TIM-3, B7-H3, VISTA, IDO, or any combination thereof. In some embodiments, the salt or composition comprises enantiomerically pure 2-S-rimantadine. In some embodiments, the salt or composition does not comprise R-rimantadine. In some embodiments, the salt or composition is formulated for an injection. In some embodiments, the methods disclosed herein comprise a method of preparing a salt of 2-S-Rimantidine comprising: dissolving 2-S-Rimantidine in a first solvent to form a first solution; adding a second solvent to the solution to form a second solution; cooling the second solution; and evaporating the second solution. In some embodiments, the first solvent is selected from a group consisting of methanol, ethanol, chloroform, and water.

### BRIEF DESCRIPTION OF THE DRAWINGS

FIGS. 1A-B shows the peak current amplitude measurements and steady state current measurements of 2-S rimantadine (TGN-S15) and 2-R rimantadine (TGN-S16) for NR2A. FIGS. 1C-D shows the peak current amplitude measurements and steady state current measurements of 2-S rimantadine (TGN-S15) and 2-R rimantadine (TGN-S16) for NR2B.

FIG. 2 shows the proliferation of CAL-27 cells with varying concentrations of RS-rimantadine (TGN-S11), S-rimantadine (TGN-S15), R-rimantadine (TGN-S16), and memantine (TGN-S13).

FIG. 3 shows an interaction map for 2-S-Rimantidine.

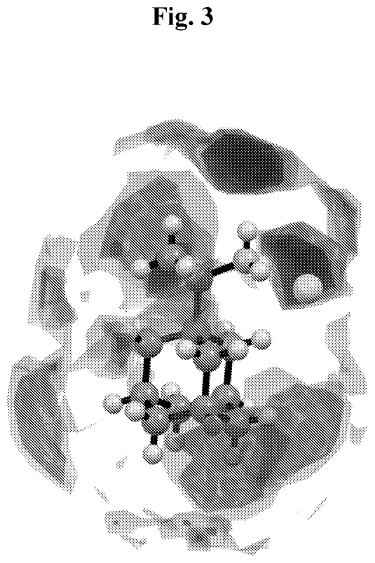

FIG. 4 shows an X-ray powder diffraction (“XRPD”) diffractogram of a Fumarate Salt Type A of 2-S-rimantidine.

FIG. 5 shows an XRPD diffractogram of a Tartrate Salt Type A of 2-S-rimantidine.

FIG. 6 shows an XRPD diffractogram of Galactarate Salt Type A of 2-S-rimantidine.

FIG. 7 shows an XRPD diffractogram of Galactarate Salt Type B of 2-S-rimantidine.

FIG. 8 shows an XRPD diffractogram of Benzoate Salt Type A of 2-S-rimantidine.

FIG. 9 shows an XRPD diffractogram of Benzenesulfonate Salt Type A of 2-S-rimantidine.

FIG. 10 shows an XRPD diffractogram of Benzenesulfonate Salt Type B of 2-S-rimantidine.

FIG. 11 shows thermogravimetric analysis and differential scanning calorimetry of Tartrate Salt Type A of 2-S-rimantidine.

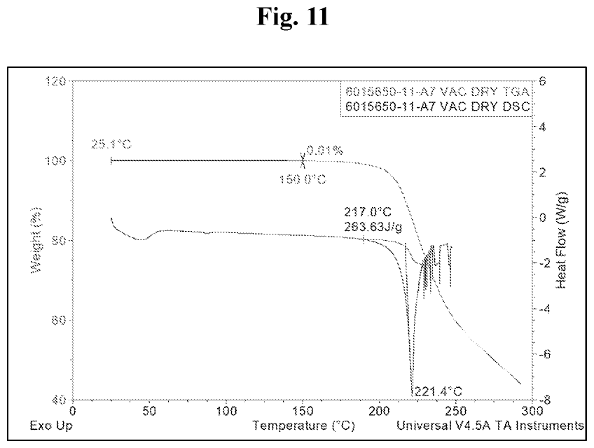

FIG. 12 shows thermogravimetric analysis and differential scanning calorimetry of Maleate Salt Type A of 2-S-rimantidine.

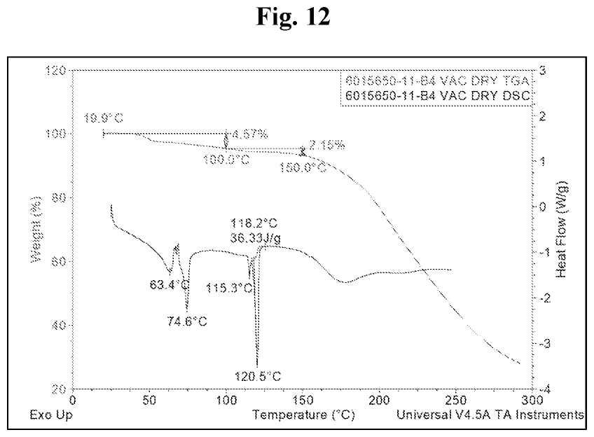

FIG. 13 shows thermogravimetric analysis and differential scanning calorimetry of Hippurate Salt Type A of 2-S-rimantidine.

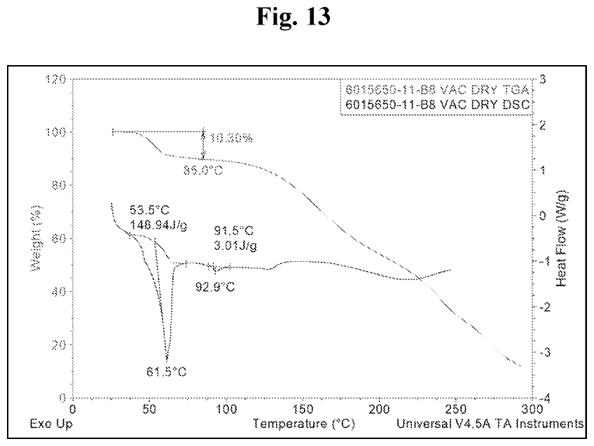

FIG. 14 shows thermogravimetric analysis and differential scanning calorimetry of Galactarate Salt Type A of 2-S-rimantidine.

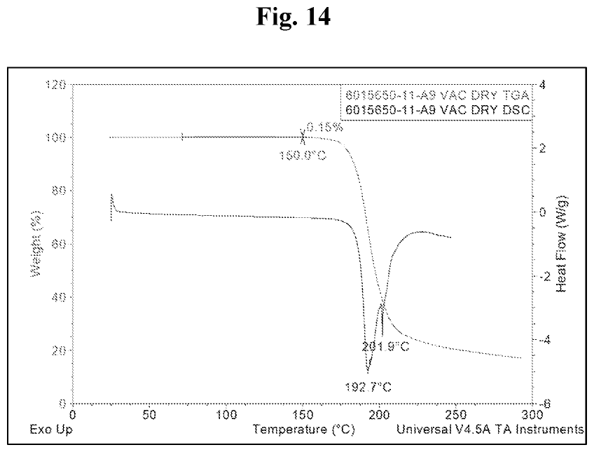

FIG. 15 shows thermogravimetric analysis and differential scanning calorimetry of Galactarate Salt Type B of 2-S-rimantidine.

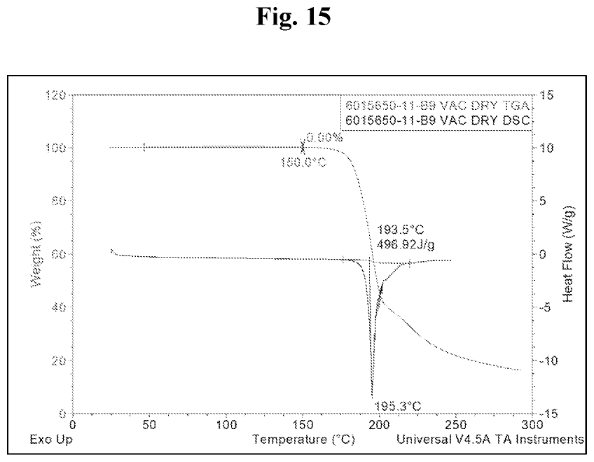

FIG. 16 shows thermogravimetric analysis and differential scanning calorimetry of Benzoate Salt Type A of 2-S-rimantidine.

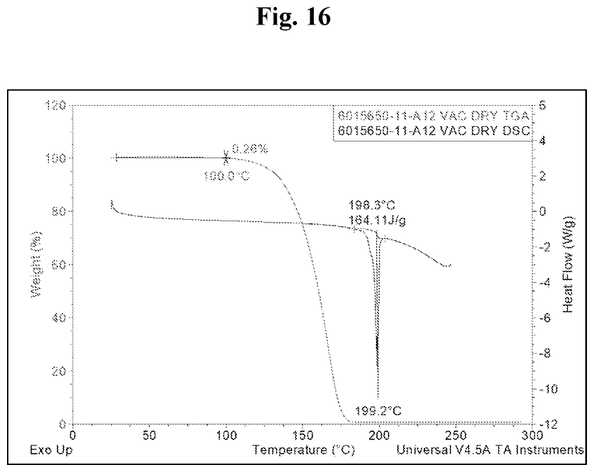

FIG. 17 shows thermogravimetric analysis and differential scanning calorimetry of Benzoate Salt Type B of 2-S-rimantidine.

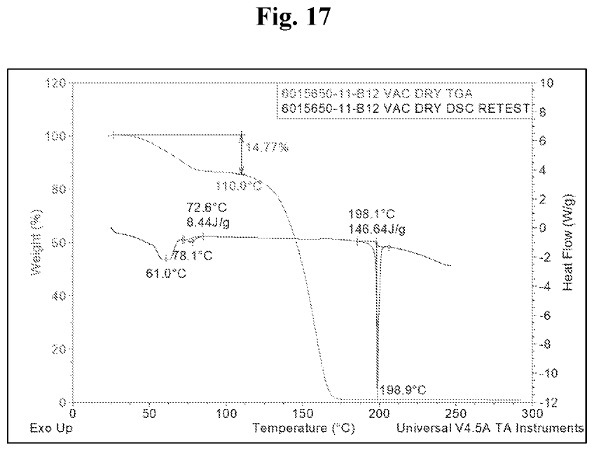

FIG. 18 shows thermogravimetric analysis and differential scanning calorimetry of Gentisate Salt Type A of 2-S-rimantidine.

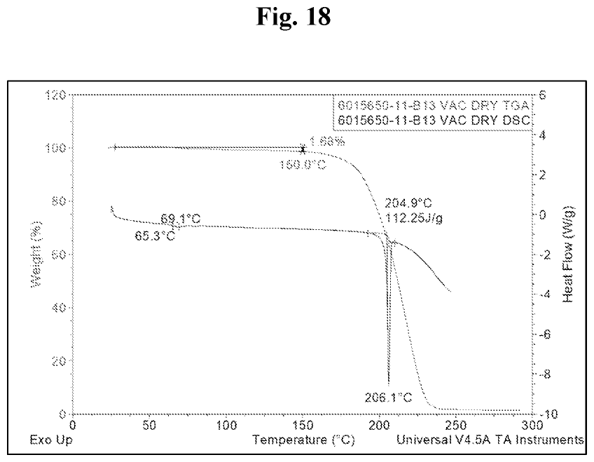

FIG. 19 shows thermogravimetric analysis and differential scanning calorimetry of Toluenesulfonate Salt Type A of 2-S-rimantidine.

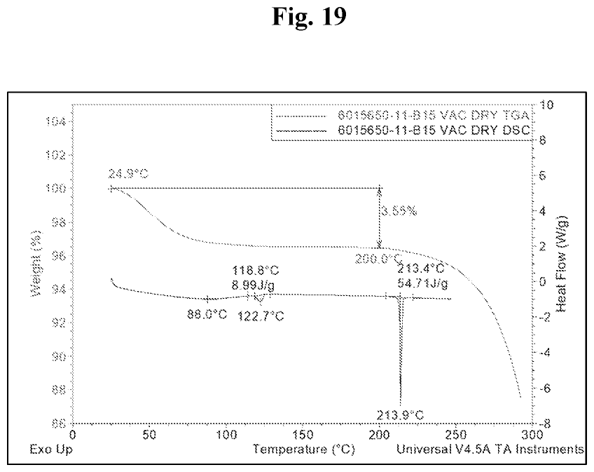

FIG. 20 shows thermogravimetric analysis and differential scanning calorimetry of Benzenesulfonate Salt Type A of 2-S-rimantidine.

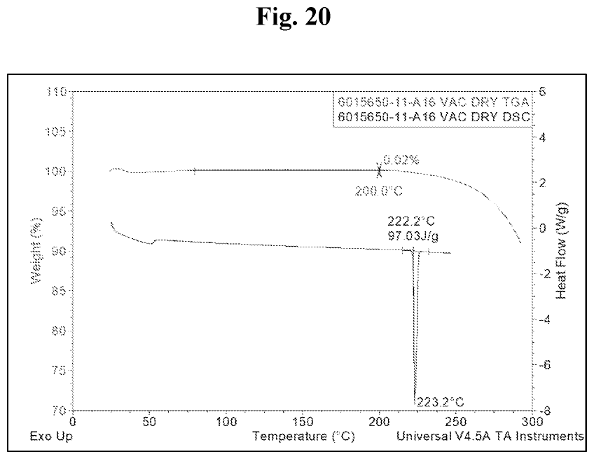

FIG. 21 shows thermogravimetric analysis and differential scanning calorimetry of Benzenesulfonate Salt Type B of 2-S-rimantidine.

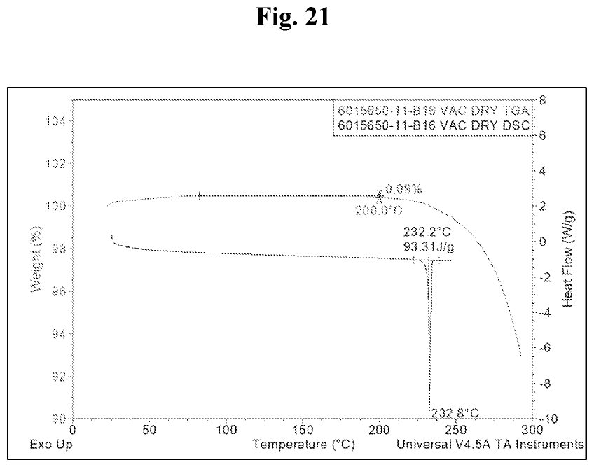

FIG. 22 shows polarized light microscopy analysis of Fumarate Salt Type A of 2-S-rimantidine.

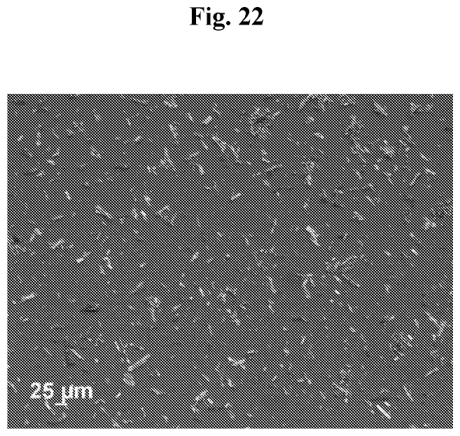

FIG. 23 shows polarized light microscopy analysis of Tartrate Salt Type A of 2-S-rimantidine.

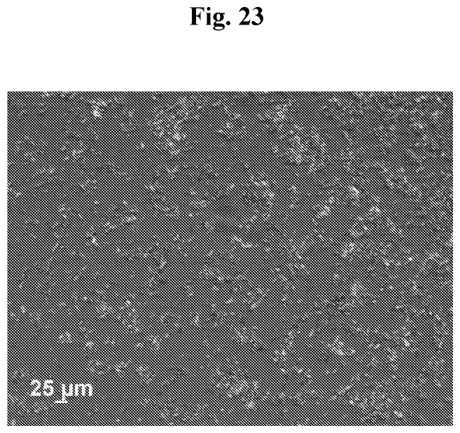

FIG. 24 shows polarized light microscopy analysis of Maleate Salt Type A of 2-S-rimantidine.

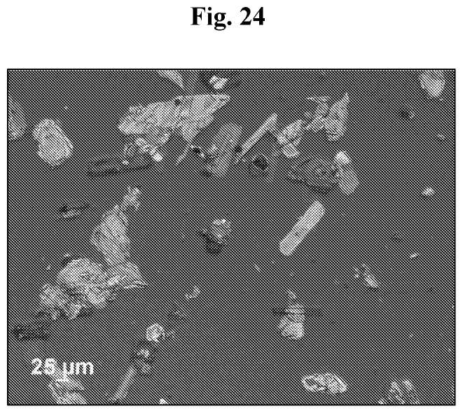

FIG. 25 shows polarized light microscopy analysis of Hippurate Salt Type A of 2-S-rimantidine.

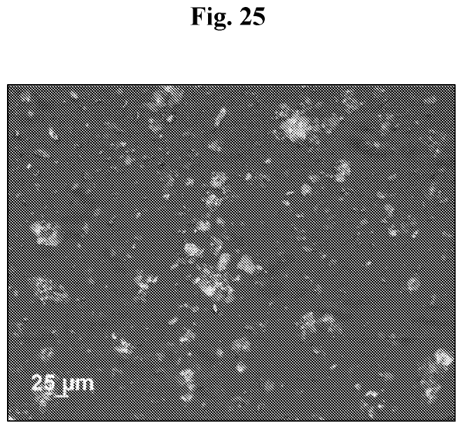

FIG. 26 shows polarized light microscopy analysis of Galactarate Salt Type A of 2-S-rimantidine.

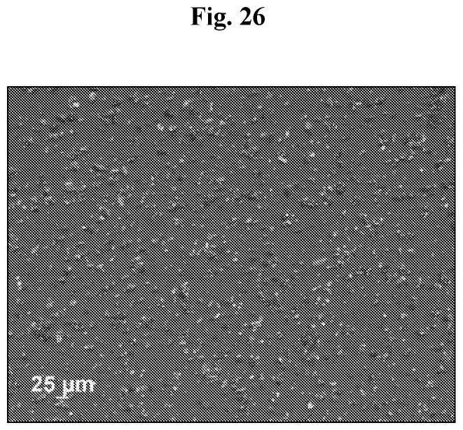

FIG. 27 shows polarized light microscopy analysis of Galactarate Salt Type B of 2-S-rimantidine.

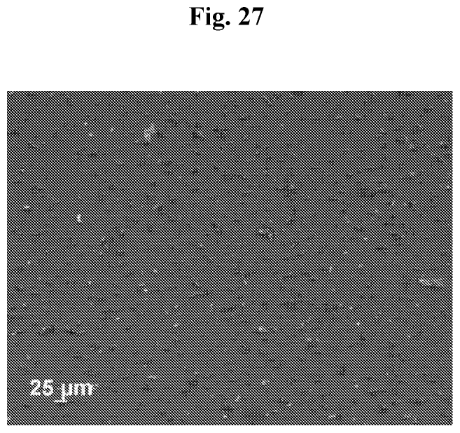

FIG. 28 shows polarized light microscopy analysis of Benzoate Salt Type A of 2-S-rimantidine.

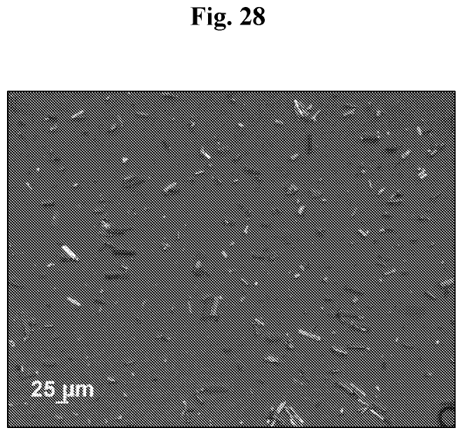

FIG. 29 shows polarized light microscopy analysis of Benzoate Salt Type B of 2-S-rimantidine.

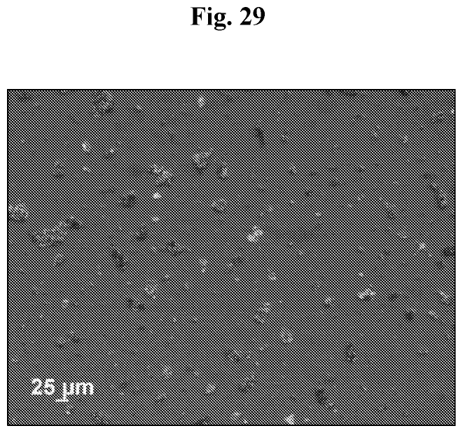

FIG. 30 shows polarized light microscopy analysis of Gentisate Salt Type A of 2-S-rimantidine.

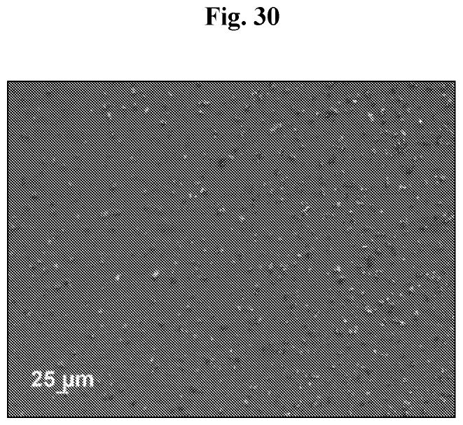

FIG. 31 shows polarized light microscopy analysis of Toluenesulfonate Salt Type A of 2-S-rimantidine.

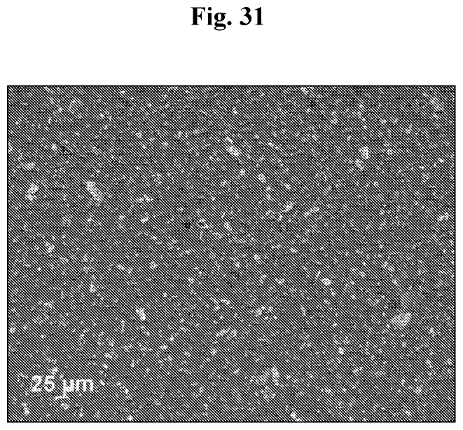

FIG. 32 shows polarized light microscopy analysis of Benzenesulfonate Salt Type A of 2-S-rimantidine.

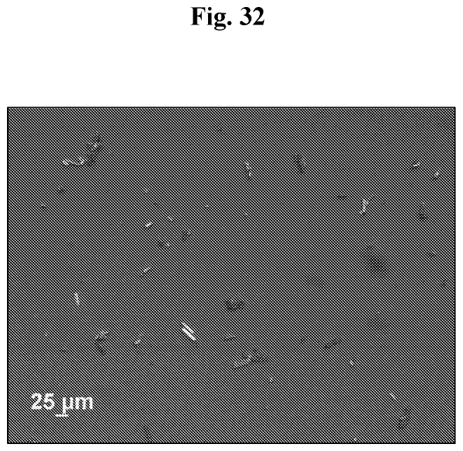

FIG. 33 shows polarized light microscopy analysis of Benzenesulfonate Salt Type B of 2-S-rimantidine.

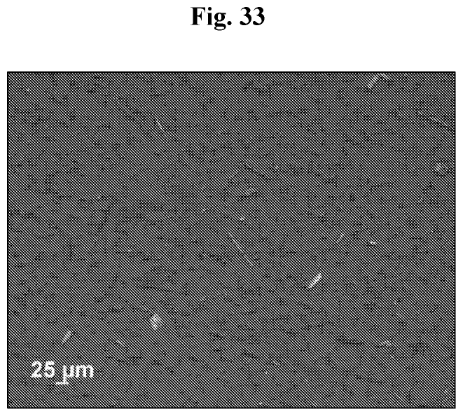

### DETAILED DESCRIPTION OF THE EMBODIMENTS

An aspect of the present disclosure is the use of enantiomerically pure 2-S rimantadine (e.g., a salt of enantiomerically pure 2-S rimantadine (e.g., a fumaric acid salt, a tartaric acid salt, a galactaric acid salt, a benzoic acid salt, a benzenesulfonic acid salt, or any combination thereof) or enantiomerically pure 2-R rimantadine for treating cancers. In some embodiments disclosed herein is the use of 2-S rimantadine (e.g., a salt of 2-S rimantadine) (also referred to as “S-rimantadine”) or enantiomerically pure 2-R rimantadine for treating cancers associated with papilloma viruses, such as human papilloma viruses (HPV). In some embodiments, the HPV is an HPV from the alpha genus.

Another aspect of the disclosure is the use of enantiomerically pure 2-S rimantadine (e.g., a salt of 2-S rimantadine) or enantiomerically pure 2-R rimantadine for treating precancerous lesions associated with papilloma viruses, such as human papilloma viruses.

Racemic rimantadine has side effects at currently prescribed doses. The side effects include central nervous system (CNS) side effects, sleep side effects, gastrointestinal side effects, and atropinic side effects, such as, without limitation light headedness, dizziness, depression, confusion, difficulty concentrating, anxiety (such as nervousness), irritability, hallucinations, and headache, insomnia, excess fatigue, loss of appetite, nausea, vomiting, constipation, dry mouth, blurred vision, difficulty voiding and difficulty swallowing. Anxiety and insomnia are the most commonly cited toxicities of racemic rimantadine for discontinuation of treatment.

As disclosed herein, 2-S rimantadine inhibits the N-methyl-D-aspartate subtype glutamate receptors (NMDA) subunit NR2B subunits to a lesser degree as compared to 2-R rimantadine and racemic rimantadine (See, Table 2 in Example 2 below).

In some embodiments, disclosed herein is the use of 2-S rimantadine (e.g., a salt of 2-S rimantadine (e.g., enantiomerically pure salt)) for treating cancer, cancer associated with HPV, precancerous lesions associated with HPV, and/or influenza A. Due to its lower ability to inhibit NR2B as compared to racemic rimantadine, 2-S rimantadine can have less side effects as compared to treating these conditions with racemic rimantadine or 2-R rimantadine. Due to its ability to inhibit NR2B to a greater degree than 2-S rimantadine, 2-R rimantadine can have less side effects as compared to treating these conditions with racemic rimantadine or 2-S rimantadine.

In some embodiments, disclosed herein is the use of 2-R rimantadine for treating cancer, cancer associated with HPV, precancerous lesions associated with HPV, and/or influenza A. Due to its greater ability to inhibit NR2B as compared to racemic rimantadine, 2-R rimantadine can have less side effects as compared to treating these conditions with racemic rimantadine or 2-S rimantadine.

In some embodiments, disclosed herein 2-S rimantadine (e.g., a salt of 2-S rimantadine (e.g., enantiomerically pure salt)) can have less side effects as compared to treating these conditions with racemic rimantadine or 2-R rimantadine due to 2-S rimantadine's lower ability to antagonize NMDA receptors and/or inhibit NMDA-mediated biological pathways. In some embodiments, the degree of NMDA receptor inhibition caused by 2-S rimantadine, with respect to 2-R rimantadine or racemic rimantadine is about 10% less to about 100% less. In some embodiments, the degree of NMDA receptor inhibition caused by 2-S rimantadine, with respect to 2-R rimantadine or racemic rimantadine is about 10% less to about 2θ% less, about 10% less to about 30% less, about 10% less to about 40% less, about 10% less to about 50% less, about 10% less to about 60% less, about 10% less to about 70% less, about 10% less to about 80% less, about 10% less to about 90% less, about 10% less to about 100% less, about 2θ% less to about 30% less, about 2θ% less to about 40% less, about 2θ% less to about 50% less, about 2θ% less to about 60% less, about 2θ% less to about 70% less, about 2θ% less to about 80% less, about 2θ% less to about 90% less, about 2θ% less to about 100% less, about 30% less to about 40% less, about 30% less to about 50% less, about 30% less to about 60% less, about 30% less to about 70% less, about 30% less to about 80% less, about 30% less to about 90% less, about 30% less to about 100% less, about 40% less to about 50% less, about 40% less to about 60% less, about 40% less to about 70% less, about 40% less to about 80% less, about 40% less to about 90% less, about 40% less to about 100% less, about 50% less to about 60% less, about 50% less to about 70% less, about 50% less to about 80% less, about 50% less to about 90% less, about 50% less to about 100% less, about 60% less to about 70% less, about 60% less to about 80% less, about 60% less to about 90% less, about 60% less to about 100% less, about 70% less to about 80% less, about 70% less to about 90% less, about 70% less to about 100% less, about 80% less to about 90% less, about 80% less to about 100% less, or about 90% less to about 100% less. In some embodiments, the degree of NMDA receptor inhibition caused by 2-S rimantadine, with respect to 2-R rimantadine or racemic rimantadine is about 10% less, about 2θ% less, about 30% less, about 40% less, about 50% less, about 60% less, about 70% less, about 80% less, about 90% less, or about 100% less. In some embodiments, the degree of NMDA receptor inhibition caused by 2-S rimantadine, with respect to 2-R rimantadine or racemic rimantadine is at least about 10% less, about 2θ% less, about 30% less, about 40% less, about 50% less, about 60% less, about 70% less, about 80% less, or about 90% less. In some embodiments, the degree of NMDA receptor inhibition caused by 2-S rimantadine, with respect to 2-R rimantadine or racemic rimantadine is at most about 2θ% less, about 30% less, about 40% less, about 50% less, about 60% less, about 70% less, about 80% less, about 90% less, or about 100% less. In some embodiments, the NMDA receptor is NR2A. In some embodiments, the NMDA receptor is NR2B.

In some embodiments, disclosed herein 2-R rimantadine can have less side effects as compared to treating these conditions with racemic rimantadine or 2-S rimantadine due to 2-R rimantadine's lower ability to antagonize NMDA receptors and/or inhibit NMDA-mediated biological pathways. In some embodiments, the degree of NMDA receptor inhibition caused by 2-R rimantadine, with respect to 2-S rimantadine or racemic rimantadine is about 10% less to about 100% less. In some embodiments, the degree of NMDA receptor inhibition caused by 2-R rimantadine, with respect to 2-S rimantadine or racemic rimantadine is about 10% less to about 2θ% less, about 10% less to about 30% less, about 10% less to about 40% less, about 10% less to about 50% less, about 10% less to about 60% less, about 10% less to about 70% less, about 10% less to about 80% less, about 10% less to about 90% less, about 10% less to about 100% less, about 2θ% less to about 30% less, about 2θ% less to about 40% less, about 2θ% less to about 50% less, about 2θ% less to about 60% less, about 2θ% less to about 70% less, about 2θ% less to about 80% less, about 2θ% less to about 90% less, about 2θ% less to about 100% less, about 30% less to about 40% less, about 30% less to about 50% less, about 30% less to about 60% less, about 30% less to about 70% less, about 30% less to about 80% less, about 30% less to about 90% less, about 30% less to about 100% less, about 40% less to about 50% less, about 40% less to about 60% less, about 40% less to about 70% less, about 40% less to about 80% less, about 40% less to about 90% less, about 40% less to about 100% less, about 50% less to about 60% less, about 50% less to about 70% less, about 50% less to about 80% less, about 50% less to about 90% less, about 50% less to about 100% less, about 60% less to about 70% less, about 60% less to about 80% less, about 60% less to about 90% less, about 60% less to about 100% less, about 70% less to about 80% less, about 70% less to about 90% less, about 70% less to about 100% less, about 80% less to about 90% less, about 80% less to about 100% less, or about 90% less to about 100% less. In some embodiments, the degree of NMDA receptor inhibition caused by 2-R rimantadine, with respect to 2-S rimantadine or racemic rimantadine is about 10% less, about 2θ% less, about 30% less, about 40% less, about 50% less, about 60% less, about 70% less, about 80% less, about 90% less, or about 100% less. In some embodiments, the degree of NMDA receptor inhibition caused by 2-R rimantadine, with respect to 2-S rimantadine or racemic rimantadine is at least about 10% less, about 2θ% less, about 30% less, about 40% less, about 50% less, about 60% less, about 70% less, about 80% less, or about 90% less. In some embodiments, the degree of NMDA receptor inhibition caused by 2-R rimantadine, with respect to 2-S rimantadine or racemic rimantadine is at most about 2θ% less, about 30% less, about 40% less, about 50% less, about 60% less, about 70% less, about 80% less, about 90% less, or about 100% less. In some embodiments, the NMDA receptor is NR2A. In some embodiments, the NMDA receptor is NR2B.

In some embodiments, 2-S rimantadine (e.g., a salt of 2-S rimantadine (e.g., enantiomerically pure salt)) can have less side effects as compared to treating these conditions with racemic rimantadine or 2-R rimantadine due to 2-S rimantadine's lower ability to antagonize GABA receptors and/or inhibit GABA-mediated biological pathways. In some embodiments, the degree of GABA receptor and/or GABA-mediated biological pathway inhibition caused by 2-S rimantadine, with respect to 2-R rimantadine or racemic rimantadine is about 10% less to about 100% less. In some embodiments, the degree of GABA receptor and/or GABA-mediated biological pathway inhibition caused by 2-S rimantadine (e.g., a salt of 2-S rimantadine), with respect to 2-R rimantadine or racemic rimantadine is about 10% less to about 2θ% less, about 10% less to about 30% less, about 10% less to about 40% less, about 10% less to about 50% less, about 10% less to about 60% less, about 10% less to about 70% less, about 10% less to about 80% less, about 10% less to about 90% less, about 10% less to about 100% less, about 2θ% less to about 30% less, about 2θ% less to about 40% less, about 2θ% less to about 50% less, about 2θ% less to about 60% less, about 2θ% less to about 70% less, about 2θ% less to about 80% less, about 2θ% less to about 90% less, about 2θ% less to about 100% less, about 30% less to about 40% less, about 30% less to about 50% less, about 30% less to about 60% less, about 30% less to about 70% less, about 30% less to about 80% less, about 30% less to about 90% less, about 30% less to about 100% less, about 40% less to about 50% less, about 40% less to about 60% less, about 40% less to about 70% less, about 40% less to about 80% less, about 40% less to about 90% less, about 40% less to about 100% less, about 50% less to about 60% less, about 50% less to about 70% less, about 50% less to about 80% less, about 50% less to about 90% less, about 50% less to about 100% less, about 60% less to about 70% less, about 60% less to about 80% less, about 60% less to about 90% less, about 60% less to about 100% less, about 70% less to about 80% less, about 70% less to about 90% less, about 70% less to about 100% less, about 80% less to about 90% less, about 80% less to about 100% less, or about 90% less to about 100% less. In some embodiments, the degree of GABA receptor and/or GABA-mediated biological pathway inhibition caused by 2-S rimantadine, with respect to 2-R rimantadine or racemic rimantadine is about 10% less, about 2θ% less, about 30% less, about 40% less, about 50% less, about 60% less, about 70% less, about 80% less, about 90% less, or about 100% less. In some embodiments, the degree of GABA receptor and/or GABA-mediated biological pathway inhibition caused by 2-S rimantadine, with respect to 2-R rimantadine or racemic rimantadine is at least about 10% less, about 2θ% less, about 30% less, about 40% less, about 50% less, about 60% less, about 70% less, about 80% less, or about 90% less. In some embodiments, the degree of GABA receptor and/or GABA-mediated biological pathway inhibition caused by 2-S rimantadine, with respect to 2-R rimantadine or racemic rimantadine is at most about 2θ% less, about 30% less, about 40% less, about 50% less, about 60% less, about 70% less, about 80% less, about 90% less, or about 100% less.

In some embodiments, disclosed herein 2-R rimantadine can have less side effects as compared to treating these conditions with racemic rimantadine or 2-S rimantadine due to 2-R rimantadine's lower ability to antagonize GABA receptors and/or inhibit GABA-mediated biological pathways. In some embodiments, the degree of GABA receptor and/or GABA-mediated biological pathway inhibition caused by 2-R rimantadine, with respect to 2-S rimantadine or racemic rimantadine is about 10% less to about 100% less. In some embodiments, the degree of GABA receptor and/or GABA-mediated biological pathway inhibition caused by 2-R rimantadine, with respect to 2-S rimantadine or racemic rimantadine is about 10% less to about 2θ% less, about 10% less to about 30% less, about 10% less to about 40% less, about 10% less to about 50% less, about 10% less to about 60% less, about 10% less to about 70% less, about 10% less to about 80% less, about 10% less to about 90% less, about 10% less to about 100% less, about 2θ% less to about 30% less, about 2θ% less to about 40% less, about 2θ% less to about 50% less, about 2θ% less to about 60% less, about 2θ% less to about 70% less, about 2θ% less to about 80% less, about 2θ% less to about 90% less, about 2θ% less to about 100% less, about 30% less to about 40% less, about 30% less to about 50% less, about 30% less to about 60% less, about 30% less to about 70% less, about 30% less to about 80% less, about 30% less to about 90% less, about 30% less to about 100% less, about 40% less to about 50% less, about 40% less to about 60% less, about 40% less to about 70% less, about 40% less to about 80% less, about 40% less to about 90% less, about 40% less to about 100% less, about 50% less to about 60% less, about 50% less to about 70% less, about 50% less to about 80% less, about 50% less to about 90% less, about 50% less to about 100% less, about 60% less to about 70% less, about 60% less to about 80% less, about 60% less to about 90% less, about 60% less to about 100% less, about 70% less to about 80% less, about 70% less to about 90% less, about 70% less to about 100% less, about 80% less to about 90% less, about 80% less to about 100% less, or about 90% less to about 100% less. In some embodiments, the degree of GABA receptor and/or GABA-mediated biological pathway inhibition caused by 2-R rimantadine, with respect to 2-S rimantadine or racemic rimantadine is about 10% less, about 2θ% less, about 30% less, about 40% less, about 50% less, about 60% less, about 70% less, about 80% less, about 90% less, or about 100% less. In some embodiments, the degree of GABA receptor and/or GABA-mediated biological pathway inhibition caused by 2-R rimantadine, with respect to 2-S rimantadine or racemic rimantadine is at least about 10% less, about 2θ% less, about 30% less, about 40% less, about 50% less, about 60% less, about 70% less, about 80% less, or about 90% less. In some embodiments, the degree of GABA receptor and/or GABA-mediated biological pathway inhibition caused by 2-R rimantadine, with respect to 2-S rimantadine or racemic rimantadine is at most about 2θ% less, about 30% less, about 40% less, about 50% less, about 60% less, about 70% less, about 80% less, about 90% less, or about 100% less.

In some embodiments, disclosed herein 2-S rimantadine (e.g., a salt of 2-S rimantadine (e.g., enantiomerically pure salt)) can have less side effects as compared to treating these conditions with racemic rimantadine or 2-R rimantadine due to 2-S rimantadine's lower ability to antagonize dopamine receptors and/or inhibit dopamine-mediated biological pathways. In some embodiments, the degree of dopamine receptor and/or dopamine-mediated biological pathway inhibition caused by 2-S rimantadine, with respect to 2-R rimantadine or racemic rimantadine is about 10% less to about 100% less. In some embodiments, the degree of dopamine receptor and/or dopamine-mediated biological pathway inhibition caused by 2-S rimantadine, with respect to 2-R rimantadine or racemic rimantadine is about 10% less to about 2θ% less, about 10% less to about 30% less, about 10% less to about 40% less, about 10% less to about 50% less, about 10% less to about 60% less, about 10% less to about 70% less, about 10% less to about 80% less, about 10% less to about 90% less, about 10% less to about 100% less, about 2θ% less to about 30% less, about 2θ% less to about 40% less, about 2θ% less to about 50% less, about 2θ% less to about 60% less, about 2θ% less to about 70% less, about 2θ% less to about 80% less, about 2θ% less to about 90% less, about 2θ% less to about 100% less, about 30% less to about 40% less, about 30% less to about 50% less, about 30% less to about 60% less, about 30% less to about 70% less, about 30% less to about 80% less, about 30% less to about 90% less, about 30% less to about 100% less, about 40% less to about 50% less, about 40% less to about 60% less, about 40% less to about 70% less, about 40% less to about 80% less, about 40% less to about 90% less, about 40% less to about 100% less, about 50% less to about 60% less, about 50% less to about 70% less, about 50% less to about 80% less, about 50% less to about 90% less, about 50% less to about 100% less, about 60% less to about 70% less, about 60% less to about 80% less, about 60% less to about 90% less, about 60% less to about 100% less, about 70% less to about 80% less, about 70% less to about 90% less, about 70% less to about 100% less, about 80% less to about 90% less, about 80% less to about 100% less, or about 90% less to about 100% less. In some embodiments, the degree of dopamine receptor and/or dopamine-mediated biological pathway inhibition caused by 2-S rimantadine, with respect to 2-R rimantadine or racemic rimantadine is about 10% less, about 2θ% less, about 30% less, about 40% less, about 50% less, about 60% less, about 70% less, about 80% less, about 90% less, or about 100% less. In some embodiments, the degree of dopamine receptor and/or dopamine-mediated biological pathway inhibition caused by 2-S rimantadine, with respect to 2-R rimantadine or racemic rimantadine is at least about 10% less, about 2θ% less, about 30% less, about 40% less, about 50% less, about 60% less, about 70% less, about 80% less, or about 90% less. In some embodiments, the degree of dopamine receptor and/or dopamine-mediated biological pathway inhibition caused by 2-S rimantadine, with respect to 2-R rimantadine or racemic rimantadine is at most about 2θ% less, about 30% less, about 40% less, about 50% less, about 60% less, about 70% less, about 80% less, about 90% less, or about 100% less. In some embodiments, the dopamine receptor is the D2/3 receptor.

In some embodiments, 2-R rimantadine can have less side effects as compared to treating these conditions with racemic rimantadine or 2-S rimantadine due to 2-R rimantadine's lower ability to antagonize dopamine receptors and/or inhibit dopamine-mediated biological pathways. In some embodiments, the degree of dopamine receptor and/or dopamine-mediated biological pathway inhibition caused by 2-R rimantadine, with respect to 2-S rimantadine or racemic rimantadine is about 10% less to about 100% less. In some embodiments, the degree of dopamine receptor and/or dopamine-mediated biological pathway inhibition caused by 2-R rimantadine, with respect to 2-S rimantadine or racemic rimantadine is about 10% less to about 2θ% less, about 10% less to about 30% less, about 10% less to about 40% less, about 10% less to about 50% less, about 10% less to about 60% less, about 10% less to about 70% less, about 10% less to about 80% less, about 10% less to about 90% less, about 10% less to about 100% less, about 2θ% less to about 30% less, about 2θ% less to about 40% less, about 2θ% less to about 50% less, about 2θ% less to about 60% less, about 2θ% less to about 70% less, about 2θ% less to about 80% less, about 2θ% less to about 90% less, about 2θ% less to about 100% less, about 30% less to about 40% less, about 30% less to about 50% less, about 30% less to about 60% less, about 30% less to about 70% less, about 30% less to about 80% less, about 30% less to about 90% less, about 30% less to about 100% less, about 40% less to about 50% less, about 40% less to about 60% less, about 40% less to about 70% less, about 40% less to about 80% less, about 40% less to about 90% less, about 40% less to about 100% less, about 50% less to about 60% less, about 50% less to about 70% less, about 50% less to about 80% less, about 50% less to about 90% less, about 50% less to about 100% less, about 60% less to about 70% less, about 60% less to about 80% less, about 60% less to about 90% less, about 60% less to about 100% less, about 70% less to about 80% less, about 70% less to about 90% less, about 70% less to about 100% less, about 80% less to about 90% less, about 80% less to about 100% less, or about 90% less to about 100% less. In some embodiments, the degree of dopamine receptor and/or dopamine-mediated biological pathway inhibition caused by 2-R rimantadine, with respect to 2-S rimantadine or racemic rimantadine is about 10% less, about 2θ% less, about 30% less, about 40% less, about 50% less, about 60% less, about 70% less, about 80% less, about 90% less, or about 100% less. In some embodiments, the degree of dopamine receptor and/or dopamine-mediated biological pathway inhibition caused by 2-R rimantadine, with respect to 2-S rimantadine or racemic rimantadine is at least about 10% less, about 2θ% less, about 30% less, about 40% less, about 50% less, about 60% less, about 70% less, about 80% less, or about 90% less. In some embodiments, the degree of dopamine receptor and/or dopamine-mediated biological pathway inhibition caused by 2-R rimantadine, with respect to 2-S rimantadine or racemic rimantadine is at most about 2θ% less, about 30% less, about 40% less, about 50% less, about 60% less, about 70% less, about 80% less, about 90% less, or about 100% less. In some embodiments, the dopamine receptor is the D2/3 receptor.

In another aspect, disclosed herein is the use of 2-S rimantadine (e.g., a salt of 2-S rimantadine (e.g., enantiomerically pure salt)) for the treatment/prevention of flu in veterinary animals, for example, poultry (e.g., chickens, turkeys, and ducks) and horses. Use of 2-S rimantadine can have less side effects as compared to treating these animals with racemic rimantadine or 2-R rimantadine.

In another aspect, disclosed herein is the use of 2-R rimantadine for the treatment/prevention of flu in veterinary animals, for example, poultry (e.g., chickens, turkeys, and ducks) and horses. Use of 2-R rimantadine can have less side effects as compared to treating these animals with racemic rimantadine or 2-S rimantadine.

### DEFINITIONS

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed invention.

In some embodiments, S-rimantadine (e.g., a salt of 2-S rimantadine), R-rimantadine, racemic rimantadine, or a rimantadine derivative as described herein is PEGylated. As used herein, “PEGylated” or “PEGylation” describes the conjugation of a compound with a polyethylene glycol (PEG) moiety. The PEG moiety can be of any length. For example, the PEG moiety can have from 2 to 500 repeating units. In some embodiments, the PEG moiety can have an average molecular weight of about 300 g/mol to about 10,000,000 g/mol. In some embodiments, the PEG moiety can be a high molecular weight PEG or low molecular weight PEG. For example, a high molecular weight PEG has a molecular weight greater than or equal to 5 kDa, and a low molecular weight PEG has a molecular weight of less than 5 kDa. In some embodiments, the PEG is selected from the group consisting of: PEG 200, PEG 300, PEG 400, PEG 600, PEG 800, PEG 1000, PEG 1500, PEG 2000, and PEG 3350. A PEG moiety can be a linear PEG or the PEG moiety can be a branched PEG. For example, branched PEGs includes any PEG having one or more branches of PEG groups extending from a PEG backbone.

As used herein the term “pure” when applied to a chiral compound, refers to an enantiomer of the chiral compound substantially free from its opposite enantiomer (i.e., in enantiomeric excess). For example, the pure “R” form of a compound is substantially free from the “S” form of the compound and is, thus, in enantiomeric excess of the “S” form. The term “enantiomerically pure” or “pure enantiomer” denotes that the compound comprises an excess of an enantiomer, e.g. more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of the compound, i.e. all enantiomers of the compound. In certain embodiments, one enantiomer can be in excess by 30-80%, or by 30-70%, 30-60%, 30%, 35%, 40%, 45%, 50%, 55% or 60%, or any percentage in between.

As used herein and unless otherwise indicated, the term “enantiomerically pure 2-S rimantadine” refers, e.g., to at least about 80% by weight 2-S rimantadine and at most about 2θ% by weight 2-R rimantadine, at least about 90% by weight 2-S rimantadine and at most about 10% by weight 2-R rimantadine, at least about 95% by weight 2-S rimantadine and at most about 5% by weight 2-R rimantadine, at least about 99% by weight 2-S rimantadine and at most about 1% by weight 2-R rimantadine or at least about 99.9% by weight 2-S rimantadine and at most about 0.1% by weight 2-R rimantadine. In certain embodiments, the weights are based upon total weight of rimantadine, i.e., both or all of the enantiomers of rimantadine.

As used herein and unless otherwise indicated, the term “enantiomerically pure “2-R rimantadine” refers, e.g., to at least about 80% by weight 2-R rimantadine and at most about 2θ% by weight 2-S rimantadine, at least about 90% by weight 2-R rimantadine and at most about 10% by weight 2-S rimantadine, at least about 95% by weight 2-R rimantadine and at most about 5% by weight 2-S rimantadine, at least about 99% by weight 2-R rimantadine and at most about 1% by weight 2-S rimantadine, at least about 99.9% by weight 2-R rimantadine or at most about 0.1% by weight 2-S rimantadine. In certain embodiments, the weights are based upon total weight of rimantadine, i.e., both or all enantiomers of the rimantadine.

In the compositions provided herein, enantiomerically pure rimantadine or a pharmaceutically acceptable salt, solvate, hydrate, ester or prodrug thereof can be present with other active or inactive ingredients. For example, a pharmaceutical composition comprising enantiomerically pure 2-S rimantadine can comprise, for example, about 90% excipient and about 10% enantiomerically pure 2-S rimantadine. In certain embodiments, the enantiomerically pure 2-S rimantadine in such compositions can, for example, comprise, at least about 99.9% by weight 2-S rimantadine and at most about 0.1% by weight 2-S rimantadine. In certain embodiments, the active ingredient can be formulated with little or no carrier, excipient or diluent.

As used herein, the terms “subject,” “individual,” or “patient,” used interchangeably, refer to any animal, including poultry, such as chickens, ducks, turkeys and mammals such as mice, rats, other rodents, rabbits, dogs, cats, swine, cattle, sheep, horses, primates, and humans. In some embodiments, the subject is a human.

As used herein, the terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

The term “preventing” as used herein means the prevention of the onset, recurrence or spread, in whole or in part, of the disease or condition as described herein, or a symptom thereof.

The term “administration” or “administering” refers to a method of giving a dosage of a compound or pharmaceutical composition to a vertebrate or invertebrate, including a mammal, a bird, a fish, or an amphibian. The preferred method of administration can vary depending on various factors, e.g., the components of the pharmaceutical composition, the site of the disease, and the severity of the disease. By “therapeutically effective amount” or “pharmaceutically effective amount” of a compound as provided herein is an amount which is sufficient to achieve the desired effect and can vary according to the nature and severity of the disease condition, and the potency of the compound. A therapeutic effect is the relief, to some extent, of one or more of the symptoms of the disease, and can include curing a disease.

“Curing” means that the symptoms of active disease are eliminated. However, certain long-term or permanent effects of the disease can exist even after a cure is obtained (such as, e.g., extensive tissue damage).

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. An “effective amount” is an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “immunotherapy” refers to an agent that modulates the immune system. In some embodiments, an immunotherapy can increase the expression and/or activity of a regulator of the immune system. In some embodiments, an immunotherapy can decrease the expression and/or activity of a regulator of the immune system. In some embodiments, an immunotherapy can recruit and/or enhance the activity of an immune cell.

### Pharmaceutically Acceptable Salts, Prodrugs, Stereoisomers and Tautomers

The pure R or S enantiomers of rimantadine ((e.g., a salt of 2-S rimantadine (e.g., a fumaric acid salt, a tartaric acid salt, a galactaric acid salt, a benzoic acid salt, a benzenesulfonic acid salt, or any combination thereof) provided herein can be administered as any salt or prodrug that upon administration to the recipient is capable of providing directly or indirectly the parent compound, or that exhibits activity itself. As used herein, the term “pharmaceutically acceptable salt” refers to a salt that retains the desired biological activity of the subject compound and exhibits minimal undesired toxicological effects. The phrase “pharmaceutically acceptable salt or prodrug” is used throughout the specification to describe any pharmaceutically acceptable form (such as an ester, amide, salt of an ester, salt of an amide or related group) of a compound that, upon administration to a patient, provides an active compound of the present disclosure. Modifications like these can affect the biological activity of the compound, in some cases increasing the activity over the parent compound. The pharmaceutically acceptable salt may be prepared in situ during the final isolation and purification of the compound, or by separately reacting the purified compound in its free acid or free base form with a suitable base or acid, respectively. In some embodiments, a pharmaceutically acceptable salt can be preferred over the respective free base or free acid because such a salt imparts greater stability or solubility to the molecule thereby facilitating formulation into a dosage form. Basic compounds are generally capable of forming pharmaceutically acceptable acid addition salts by treatment with a suitable acid. Suitable acids include pharmaceutically acceptable inorganic acids and pharmaceutically acceptable organic acids. In some embodiments, a pharmaceutically acceptable acid addition salt includes fumaric acid, tartaric acid, galactaric acid, benzoic acid, benzenesulfonic acid, or any combination thereof. In some embodiments, a pharmaceutically acceptable acid addition salt includes fumaric acid. In some embodiments, a pharmaceutically acceptable acid addition salt includes tartaric acid. In some embodiments, a pharmaceutically acceptable acid addition salt includes galactaric acid. In some embodiments, a pharmaceutically acceptable acid addition salt includes benzoic acid. In some embodiments, a pharmaceutically acceptable acid addition salt includes benzenesulfonic acid. Further non-limiting examples of a pharmaceutically acceptable acid addition salt include hydrochloride, hydrobromide, nitrate, methylnitrate, sulfate, bisulfate, sulfamate, phosphate, acetate, hydroxyacetate, phenylacetate, propionate, butyrate, isobutyrate, valerate, maleate, hydroxymaleate, acrylate, fumarate, malate, tartrate, citrate, salicylate, p-aminosalicyclate, glycollate, lactate, heptanoate, phthalate, oxalate, succinate, benzoate, o-acetoxybenzoate, chlorobenzoate, methylbenzoate, dinitrobenzoate, hydroxybenzoate, methoxybenzoate, mandelate, tannate, formate, stearate, ascorbate, palmitate, oleate, pyruvate, pamoate, malonate, laurate, glutarate, glutamate, estolate, methanesulfonate (mesylate), ethanesulfonate (esylate), 2-hydroxyethanesulfonate, benzenesulfonate (besylate), p-aminobenzenesulfonate, p-toluenesulfonate (tosylate), napthalene-2-sulfonate, ethanedisulfonate, 2,5-dihydroxybenzoate, and/or any combination thereof.

In some embodiments, the rimantadine salt (e.g., 2-S-rimantadine salt) is synthesized via mixing of a slurry of starting materials. In some embodiments, the salt is synthesized at 0 °C. In some embodiments, the salt is synthesized at 5 °C. In some embodiments, the salt is synthesized by a temperature cycling method. In some embodiments, the salt is synthesized by a slow evaporation method. In some embodiments, the salt is synthesized by solvation of 2-S-Rimantidine with methanol, ethanol, chloroform, acetone, isopropyl or water, or any combinations thereof. In some embodiments, the salt is synthesized using an anti-solvent such as acetone, n-heptane, or methyl isobutyl ketone.

A pharmaceutically acceptable prodrug refers to a compound that is metabolized (i.e., hydrolyzed or oxidized, for example) in the host to form a compound of the present disclosure. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, and/or dephosphorylated to produce the active compound.

In some embodiments, a method as described herein comprises administering pure 2-S rimantadine, or pure 2-R rimantadine or a pharmaceutically acceptable salt thereof.

In some embodiments, the PK properties of the 2-S-Rimantidine are improved in salt form. In some embodiments, the salt of 2-S-Rimantidine increases AUC as compared to 2-S-Rimantidine in a non-salt form.

In certain embodiments, a sample containing a 2-S-Rimantidine solid form, such as a crystal, provided herein may be substantially free of other solid forms such as the amorphous form and/or other crystal forms. In certain embodiments, such a sample containing a crystal form of a 2-S-Rimantidine salt provided herein may contain less than 1%, 2%, 3%, 4%, 5%, 6%, 7%, 8%, 9%, 10%, 15%, 2θ%, 25%, 30%, 35%, 40%, 45% or 50% of one or more other solid forms of 2-S-Rimantidine salts such as the amorphous form.

In some embodiments, XRPD is measured by a Malvern-Panalytical Aeris X-ray powder diffractometer. In some embodiments the differential scanning calorimetry is measured by TA 2500 DSC instrument. In some embodiments, thermogravimetric analysis is measured by a TA Discovery 550 instrument.

In some embodiments of the crystalline salt, the average crystal particle size is between about 5 to about 45 μm. In some embodiments of the crystalline salt, the average crystal particle size is between about 5 to about 40 μm. In some embodiments of the crystalline salt, the average crystal particle size is between about 5 to about 35 μm. In some embodiments of the crystalline salt, the average crystal particle size is between about 5 to about 30 μm. In some embodiments of the crystalline salt, the average crystal particle size is between about 5 to about 25 μm. In some embodiments of the crystalline salt, the average crystal particle size is between about 5 to about 20 μm. In some embodiments of the crystalline salt, the average crystal particle size is between about 5 to about 15 μm. In some embodiments of the crystalline salt, the average crystal particle size is between about 5 to about 10 μm. In some embodiments of the crystalline salt, the average crystal particle size is between about 1 to about 35 μm. In some embodiments of the crystalline salt, the average crystal particle size is between about 1 to about 30 μm. In some embodiments of the crystalline salt, the average crystal particle size is between about 1 to about 25 μm. In some embodiments of the crystalline salt, the average crystal particle size is between about 1 to about 20 μm. In some embodiments of the crystalline salt, the average crystal particle size is between about 1 to about 15 μm. In some embodiments of the crystalline salt, the average crystal particle size is between about 1 to about 10 μm. In some embodiments of the crystalline salt, the average crystal particle size is between about 1 to about 5 μm. In some embodiments of the crystalline salt, the average crystal particle size is about 1 μm. In some embodiments of the crystalline salt, the average crystal particle size is about 2 μm. In some embodiments of the crystalline salt, the average crystal particle size is about 5 μm. In some embodiments of the crystalline salt, the average crystal particle size is about 10 μm. In some embodiments of the crystalline salt, the average crystal particle size is about 15 μm. In some embodiments of the crystalline salt, the average crystal particle size is about 20 μm. In some embodiments of the crystalline salt, the average crystal particle size is about 25 μm. In some embodiments of the crystalline salt, the average crystal particle size is about 30 μm. In some embodiments of the crystalline salt, the average crystal particle size is about 35 μm. In some embodiments of the crystalline salt, the average crystal particle size is about 40 μm. In some embodiments of the crystalline salt, the average crystal particle size is about 50 μm.

### Pharmaceutical Compositions

Also provided herein are pharmaceutical compositions comprising pure 2-S rimantadine, pure 2-R rimantadine or pharmaceutically acceptable salt thereof (e.g., a salt of 2-S rimantadine (e.g., a fumaric acid salt, a tartaric acid salt, a galactaric acid salt, a benzoic acid salt, a benzenesulfonic acid salt, or any combination thereof)), as described herein. Any of the pharmaceutical compositions described herein can be administered to a subject to treat a cancer as described herein.

Administration of 2-S rimantadine, pure 2-R rimantadine or the pharmaceutically acceptable salts thereof, or pharmaceutical compositions thereof, can be via any of the accepted modes of administration, including, but not limited to, orally, subcutaneously, intravenously, intranasally, topically, transdermally, intraperitoneally, intramuscularly, intrapulmonarilly, vaginally, rectally, ontologically, neurotologically, intraocularly, subconjuctivally, via anterior eye chamber injection, intravitreally, intraperitoneally, intrathecally, intracystically, intrapleurally, via wound irrigation, intrabuccally, intra-abdominally, intra-articularly, intra-aurally, intrabronchially, intracapsularly, intrameningeally, via inhalation, via endotracheal or endobronchial instillation, via direct instillation into pulmonary cavities, intraspinally, intrasynovially, intrathoracically, via thoracostomy irrigation, epidurally, intratympanically, intracisternally, intravascularly, intraventricularly, intraosseously, via irrigation of infected bone, or via application as part of any admixture with a prosthetic devices. In some embodiments, the administration method includes oral or parenteral administration.

Pharmaceutically acceptable compositions can include solid, semi-solid, liquid, solutions, colloidal, liposomes, emulsions, suspensions, complexes, coacervates and aerosols. Pharmaceutically acceptable compositions can also include dosage forms, such as, e.g., tablets, capsules, powders, liquids, suspensions, suppositories, aerosols, implants, controlled release or the like. 2-S rimantadine, pure 2-R rimantadine or the pharmaceutically acceptable salts thereof, or pharmaceutical compositions thereof, can also be administered in sustained or controlled release dosage forms, including depot injections, osmotic pumps, pills (tablets and or capsules), transdermal (including electrotransport) patches, implants and the like, for prolonged and/or timed, pulsed administration at a predetermined rate.

In some embodiments, the pharmaceutical composition is a tablet. In some embodiments, the pharmaceutical composition is a film-coated tablet.

Pure 2-S rimantadine, pure 2-R rimantadine or the pharmaceutically acceptable salts thereof, or pharmaceutical compositions thereof (e.g., a salt of 2-S rimantadine), can be administered either alone or in combination with a conventional pharmaceutical carrier, excipient or the like. Pharmaceutically acceptable excipients include, but are not limited to, ion exchangers, alumina, aluminum stearate, lecithin, self-emulsifying drug delivery systems (SEDDS) such as d-α-tocopherol, polyethylene glycol 1000, succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins can also be used to enhance delivery of compounds described herein.

In some embodiments, a pharmaceutical composition described herein will take the form of a unit dosage form such as a pill or tablet and thus the composition may contain, along with 2-S rimantadine, pure 2-R rimantadine or the pharmaceutically acceptable salt thereof (e.g., a salt of 2-S rimantadine), a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which 2-S rimantadine, pure 2-R rimantadine or the pharmaceutically acceptable salts thereof, provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

In some embodiments, the rimantadine, or pharmaceutically acceptable salt thereof, is PEGylated. In some embodiments, the PEGylated rimantadine, or pharmaceutically acceptable salt thereof, comprises a high molecular weight PEG. In some embodiments, the PEGylated rimantadine, or pharmaceutically acceptable salt thereof, comprises a low molecular weight PEG. In some embodiments, the rimantadine, or a pharmaceutically acceptable salt thereof, is modified. In some embodiments, the modification is PEGylation.

In some embodiments, the PEGylated rimantadine, or a pharmaceutically acceptable salt thereof, is PEGylated with a high molecular weight PEG. In some embodiments, the PEGylated rimantadine, or a pharmaceutically acceptable salt thereof, is PEGylated with a low molecular weight PEG. Accordingly, also provided herein are methods of treating cancer in a subject in need thereof, the method comprising administering to the subject a therapeutically effective amount of PEGylated rimantadine, or a pharmaceutically acceptable salt thereof.

In some embodiments, the pharmaceutical composition includes one or more excipients selected from the group consisting of: hypromellose, magnesium stearate, microcrystalline 5 cellulose, and sodium starch glycolate.

Liquid pharmaceutically administrable compositions can, for example, be prepared by dissolving, dispersing, etc. a compound provided herein and optional pharmaceutical adjuvants in a carrier (e.g., water, saline, aqueous dextrose, glycerol, glycols, ethanol or the like) to form a solution, colloid, liposome, emulsion, complexes, coacervate or suspension. If desired, the pharmaceutical composition can also contain minor amounts of nontoxic auxiliary substances such as wetting agents, emulsifying agents, co-solvents, solubilizing agents, pH buffering agents and the like (e.g., sodium acetate, sodium citrate, cyclodextrin derivatives, sorbitan monolaurate, triethanolamine acetate, triethanolamine oleate, and the like).

Dosage forms or compositions containing 2-S rimantadine, pure 2-R rimantadine or a pharmaceutically acceptable salt thereof (e.g., a salt of 2-S rimantadine), as described herein in the range of 0.005% to 100% with the balance made up from nontoxic carrier may be prepared. The contemplated compositions may contain 0.001%-100% of a compound provided herein, in one embodiment 0.1-95%, in another embodiment 75-85%, in a further embodiment 2θ-80%. Actual methods of preparing such dosage forms are known, or will be apparent, to those skilled in this art; for example, see Remington: The Science and Practice of Pharmacy, 22nd Edition (Pharmaceutical Press, London, U K. 2012).

The pharmaceutical compositions herein can contain, per unit dosage unit, e.g., tablet, capsule, suspension, solution, sachet for reconstitution, powder, injection, I.V., suppository, sublingual/buccal film, teaspoonful and the like, from about 0.1-1000 mg of 2-S rimantadine, pure 2-R rimantadine or pharmaceutically acceptable salt thereof (e.g., a salt of 2-S rimantadine). Pure 2-S rimantadine, pure 2-R rimantadine or pharmaceutically acceptable salt thereof, can be given at a dosage of from about 0.01-300 mg/kg/day, or any range therein, preferably from about 0.5-50 mg/kg/day, or any range therein. In some embodiments, the pharmaceutical compositions provided herein contain, per unit dosage unit, about 25 mg to about 500 mg of a compound provided herein (for example, about 25 mg to about 400 mg, about 25 mg to about 300 mg, about 25 mg to about 250 mg, about 25 mg to about 200 mg, about 25 mg to about 150 mg, about 25 mg to about 100 mg, about 25 mg to about 75 mg, about 50 mg to about 500 mg, about 100 mg to about 500 mg, about 150 mg to about 500 mg, about 200 mg to about 500 mg, about 250 mg to about 500 mg, about 300 mg to about 500 mg, about 400 mg to about 500 mg, about 50 to about 200 mg, about 100 to about 250 mg, about 50 to about 150 mg). In some embodiments, the pharmaceutical compositions provided herein 5 contain, per unit dosage unit, about 25 mg, about 50 mg, about 100 mg, about 150 mg, about 200 mg, about 250 mg, about 300 mg, about 400 mg, or about 500 mg of a compound provided herein. The dosages, however, may be varied depending upon the requirement of the patients, the severity of the condition being treated and the compound being employed. In some embodiments, the dosages are administered once daily (QD) or twice daily (BID).

In some embodiments, the present disclosure comprises a composition comprising pure 2-R rimantadine or a pharmaceutically acceptable salt thereof, pure 2-S rimantadine or a pharmaceutically acceptable salt thereof

### Methods of Treatment

Also provided herein are methods of treating cancer in a subject. In some embodiments, the method comprises administering to the subject a therapeutically effective amount of one or more of the pharmaceutical compositions described herein. In some embodiments, the pharmaceutical compositions comprise either enantiomerically pure 2-S rimantadine or a pharmaceutically acceptable salt thereof (e.g., a salt of 2-S rimantadine). In some embodiments, the pharmaceutical compositions comprise either enantiomerically pure 2-R rimantadine or a pharmaceutically acceptable salt thereof. In some embodiments, a pharmaceutically acceptable acid addition salt includes fumaric acid, tartaric acid, galactaric acid, benzoic acid, benzenesulfonic acid, or any combination thereof. In some embodiments, a pharmaceutically acceptable acid addition salt includes fumaric acid. In some embodiments, a pharmaceutically acceptable acid addition salt includes tartaric acid. In some embodiments, a pharmaceutically acceptable acid addition salt includes galactaric acid. In some embodiments, a pharmaceutically acceptable acid addition salt includes benzoic acid. In some embodiments, a pharmaceutically acceptable acid addition salt includes benzenesulfonic acid. In some embodiments, the pharmaceutically acceptable salt is a hydrochloride salt.

In some embodiments, the cancer is a sarcoma, carcinoma, melanoma, lymphoma, or leukemia. Non-limiting examples of a sarcoma include: bone sarcoma (e.g., angiosarcoma, fibrosarcoma, liposarcoma, chondrosarcoma, chordoma, Ewing's sarcoma, giant cell tumor, osteosarcoma, rhabdomyosarcoma, and synovial sarcoma) and soft tissue sarcoma (e.g., fibrosarcoma, 5 gastrointestinal stromal tumor (GIST), Kaposi's sarcoma, leiomyosarcoma, liposarcoma, rhabdomyosarcoma, and soft tissue Ewing's sarcoma). Non-limiting examples of a carcinoma include: basal cell carcinoma, squamous cell carcinoma, renal cell carcinoma, invasive ductal carcinoma, hepatocellular carcinoma, and adenocarcinoma. Non-limiting examples of lymphoma include: Non-Hodgkin's lymphoma (e.g., B-cell lymphoma, T-cell lymphoma, Burkitt's lymphoma, follicular lymphoma, mantle cell lymphoma, primary mediastinal B-cell lymphoma, small lymphocytic lymphoma, Waldenstrom macroglobulinemia) and Hodgkin's lymphoma (e.g., lymphocyte-depleted Hodgkin's disease, lymphocyte-rich Hodgkin's disease, mixed cellularity Hodgkin's lymphoma, nodular lymphocyte-predominant Hodgkin's disease, and nodular sclerosis Hodgkin's lymphoma). Non-limiting examples of leukemia include: acute hairy cell leukemia, acute lymphocytic leukemia, acute myeloid leukemia, acute promyelocytic leukemia, chronic lymphocytic leukemia, chronic myeloid leukemia, a myeloproliferative neoplasm, and systemic mastocytosis.

In some embodiments, the cancer is selected from the group consisting of: melanoma, head and neck cancer, lung cancer, colon cancer, anal cancer, breast cancer, esophageal cancer, pancreatic cancer, prostate cancer, cervical cancer, hepatic cancer, and stomach cancer.

In some embodiments, the cancer is a carcinoma. In some embodiments, the carcinoma is selected from the group consisting of: an adenocarcinoma, a squamous cell carcinoma, a transitional cell carcinoma, a hepatocellular carcinoma, and a clear cell carcinoma. In some embodiments, the cancer is a squamous cell carcinoma. In some embodiments, the squamous cell carcinoma is head and neck squamous cell carcinoma. In some embodiments, the cancer is a hepatocellular carcinoma.

In some embodiments, the cancer is selected from the group consisting of head and neck cancer, breast cancer, and melanoma.

In some embodiments, pure 2-S rimantadine or pure 2-R rimantadine, or pharmaceutically acceptable salt thereof (e.g., a salt of 2-S rimantadine), as described herein can be used to treat a hepatitis B virus (HBV)-associated cancer in a subject. An “HBV-associated cancer” as used herein is a cancer in which one or more of the cancerous cells express at least one HBV protein (for example, see, Liu et al., Hepatitis B Virus X Protein Induces RHAMM-Dependent Motility in Hepatocellular Carcinoma Cells via PI3K-Akt-Oct-1 Signaling. Mol Cancer Res. 2020 March; 18 (3): 375-389. doi: 10.1158/1541-7786. MCR-19-0463. Epub 2019 Dec. 2. PMID: 31792079). For example, one or more cancerous cells can express an HBV oncoprotein. In some embodiments, the HBV-associated cancer is a hepatic cancer (e.g., hepatocellular carcinoma). In some embodiments, the HBV-associated cancer is cervical cancer.

In some embodiments, pure 2-S rimantadine or pure 2-R rimantadine, or pharmaceutically acceptable salt thereof (e.g., a salt of 2-S rimantadine), as described herein can be used to treat a human papillomavirus (HPV)-associated cancer in a subject. An “HPV-associated cancer” as used herein is a cancer in which one or more of the cancerous cells express at least one HPV protein. For example, one or more of the cancerous cells can express a HPV oncoprotein. Human papillomavirus (HPV) can cause malignant transformation by, for example, targeting the critical tumor suppressors p53 and Rb (see, e.g., Conway and Meyers. J Dent Res. 2009 April; 88(4): 307-17; and Hoppe-Seyler. Trends Microbiol. 2018 February; 26(2): 158-168). HPV genes can also help HPV-infected cells evade immune responses (see, e.g., Senba. Oncol Rev. 5 2012 Oct. 5; 6(2): e17). For example, HPV genes and proteins can target the antigen processing and antigen presentation required for effective adaptive immune responses (see, e.g., Senba. Oncol Rev. 2012 Oct. 5; 6(2): e17; and O'Brien and Saveria Campo. Virus Res. 2002 September; 88(1-2): 103-17). There are many HPV oncoproteins including, but not limited to, HPV16 E5, E6, and E7. For example, HPV E5 is protein that has been reported to have multiple functions including regulation of tumor cell differentiation and apoptosis, modulation of H+ ATPase responsible for acidification of late endosomes, and immune modulation including direct binding and downregulation of major histocompatibility complex (MHC) class I and MHC class II (see e.g., Venuti. Mol Cancer. 2011, 10:140), which can affect antigen processing and presentation.

In some embodiments, one or more cancer cells from the subject express an HPV protein. In some embodiments, the HPV protein is one or more of an HPV E5, E6, or E7 protein. In some embodiments, the HPV E5, E6, or E7 protein is from one or more HPV subtypes selected from the group consisting of: HPV 6, HPV 11, HPV 16, HPV 18, HPV 31, HPV 33, HPV 35, HPV 39, HPV 45, HPV 51, HPV 52, HPV 56, HPV 58, HPV 66, and HPV 69. In some embodiments, the HPV protein is HPV16 E5. In some embodiments, the subject has a cancer selected from the group consisting of: head and neck cancer, a mucosal squamous cell carcinoma, a cutaneous squamous cell carcinoma, cervical cancer, vaginal cancer, vulvar cancer, penile cancer, and anal cancer.

In some embodiments, the cancer is HPV-associated cancer. In some embodiments, the HPV-associated cancer is HPV-associated head and neck squamous cell carcinoma (HNSCC).

In some embodiments pure 2-S rimantadine, or pure 2-R rimantadine or pharmaceutically acceptable salt thereof (e.g., a salt of 2-S rimantadine), as described herein can be used to treat a human papillomavirus precancerous lesion such as those associated with, without limitation, proliferative verrucous Leukoplakia (PVI), oral leukoplakia, nicotine palatinus in reverse smokers, oral erythroplakia, laryngeal keratosis, actinic cheilosis, smooth thick leukoplakia, smooth, red tongue of plummer-vinson, smokeless tobacco keratosis, syndrome oral submucous fibrosis, erythroleukoplakia, granular leukoplakia, oral lichen planus (erosive forms), smooth thin leukoplakia, nicotine stomatitis, and tobacco pouch keratosis, cervix (cervical dysplasia); and penile intraepithelial neoplasia (PeIN lesions). In the oral cavity, 24 types of HPV (1, 2, 3, 4, 6, 7, 10, 11, 13, 16, 18, 30, 31, 32, 33, 35, 45, 52, 55, 57, 59, 69, 72 and 73) have been associated with benign lesions and 12 types (2, 3, 6, 11, 13, 16, 18, 31, 33, 35, 52 and 57) with malignant lesions. Approximately 40% of invasive penile carcinomas are attributable to HPV 16, 18 and 6/11. HPV types associated with cervical oncogenicity are 15 high risk types (HPV 16, 18, 31, 33, 35, 39, 45, 51, 52, 56, 58, 59, 68, 73 and 82) and 3 “possibly high-risk types” (HPV 26, 53 and 66).

Testing for HPV is known in the art for example see Coultlee, F., et. al., 2005, Can J Infect Dis Med Microbiol 16(2): 83-91; careHPV Test Kit (QIAGEN, Redwood City, CA); Tang, K. D., 2019, Unlocking the Potential of Saliva-Based Test to Detect HPV-16-Driven Oropharyngeal Cancer, Cancers (Basel), 11 (4): 473; HPV probes (BIOCARE MEDICAL, Pacheo, CA)

In some embodiments, of the methods described herein, pure 2-S rimantidine, or pure 2-R rimantadine or pharmaceutically acceptable salt thereof (e.g., a salt of 2-S rimantadine), is administered in combination with a therapeutically effective amount of at least one additional therapeutic agent selected from one or more additional anti-cancer therapies or therapeutic agents (e.g., chemotherapeutic agents). Using a combination of different forms of treatment to treat a subject with cancer is a common practice in medical oncology. These other form(s) of conjoint treatment or therapy, in addition to 2-S rimantadine, or pharmaceutically acceptable salt thereof described herein, can include, for example, surgery, radiotherapy, and additional anti-cancer agents, such as kinase inhibitors, signal transduction inhibitors, platinum-based chemotherapy, and/or monoclonal antibodies. In some embodiments, the method further comprises administering an additional anti-cancer agent.

Non-limiting examples of additional anti-cancer agents include: carboplatin, cisplatin, gemcitabine, methotrexate, paclitaxel, pemetrexed, lomustine, temozolomide, and dacarbazine.

In some embodiments, the additional anti-cancer agent is an immunotherapy. Many types of immunotherapies can be used in combination with pure 2-S rimantadine, or pure 2-R rimantadine or pharmaceutically acceptable salts thereof, described herein. Non-limiting examples of an immunotherapy include: immune checkpoint inhibitors, antibody therapy, cellular immunotherapy, antibody-drug conjugates, cytokine therapy, mRNA-based immunotherapy, and cancer vaccines.

In some embodiments, the immunotherapy is one or more immune checkpoint inhibitors. In some embodiments, the immune checkpoint inhibitor targets one or more of: CTLA-4, PD-1, PD-L1, BTLA, LAG-3, A2AR, TIM-3, B7-H3, VISTA, and IDO. In some embodiments, the checkpoint inhibitor is selected form the group consisting of: ipilimumab, nivolumab, pembrolizumab, atezolizumab, avelumab, durvalumab, cemiplimab, tremelimumab, and a combination thereof.

In some embodiments, the immune checkpoint inhibitor is a CTLA-4 inhibitor, a PD-1 inhibitor or a PD-L1 inhibitor. In some embodiments, the CTLA-4 inhibitor is ipilimumab (YERVOY®) or tremelimumab (CP-675,206). In some embodiments, the PD-1 inhibitor is pembrolizumab (KEYTRUDA®), cemiplimab (LIBTAYO®), or nivolumab (OPDIVO®). In some embodiments, the PD-L1 inhibitor is atezolizumab (TECENTRIQ®), avelumab (BAVENCIO®) or durvalumab (IMFINZI™).

In some embodiments, the antibody therapy is bevacizumab (MVASTI™, AVASTIN®), trastuzumab (HERCEPTIN®), avelumab (BAVENCIO®), rituximab (MABTHERA™, RITUXAN®), edrecolomab (Panorex), daratumuab (DARZALEX®), olaratumab (LARTRUVO™), ofatumumab (ARZERRAR), alemtuzumab (CAMPATH®), cetuximab (ERBITUX®), oregovomab, pembrolizumab (KEYTRUDA®), dinutiximab (UNITUXIN®), obinutuzumab (GAZYVAR), tremelimumab (CP-675,206), ramucirumab (CYRAMZA®), ublituximab (TG-1101), panitumumab (VECTIBIX®), elotuzumab (EMPLICITI™), avelumab (BAVENCIO®), necitumumab (PORTRAZZA™), cirmtuzumab (UC-961), ibritumomab (ZEVALIN®), isatuximab (SAR650984), nimotuzumab, fresolimumab (GC1008), lirilumab (INN), 5 mogamulizumab (POTELIGEO®), ficlatuzumab (AV-299), denosumab (XGEVA®), ganitumab, urelumab, pidilizumab or amatuximab.

In some embodiments, the immunotherapy is a cellular immunotherapy (e.g., adoptive T-cell therapy, dendritic cell therapy, natural killer cell therapy).

In some embodiments, the immunotherapy is an antibody-drug conjugate. In some embodiments, the antibody-drug conjugate is gemtuzumab ozogamicin (MYLOTARG™), inotuzumab ozogamicin (BESPONSA®), brentuximab vedotin (ADCETRIS®), ado-trastuzumab emtansine (TDM-1; KADCYLA®), moxetumomab pasudotox (LUMOXITI®), polatuzumab vedotin-piiq (POLIVY®), mirvetuximab soravtansine (IMGN853), or anetumab ravtansine.

In some embodiments, the immunotherapy is a cytokine therapy. In some embodiments, the cytokine therapy is an interleukin 2 (IL-2) therapy, an interleukin (IL-15) therapy, an interleukin 7 (IL-7) therapy, an interferon alpha (IFNα) therapy, agranulocyte colony stimulating factor (G-CSF) therapy, an interleukin 12 (IL-12) therapy, or an erythropoietin-alpha (EPO) therapy. In some embodiments, the IL-2 therapy is aldesleukin (Proleukin®). In some embodiments, the IFNα therapy is interferon alfa-2b (e.g., IntronAR) or interferon alfa-2a (e.g., Roferon-AR). In some embodiments, the G-CSF therapy is filgrastim (Neupogen®).

In some embodiments, the immunotherapy is mRNA-based immunotherapy. In some embodiments, the mRNA-based immunotherapy is CV9104 (see, e.g., Rausch et al. (2014) Human Vaccin Immunother 10(11): 3146-52; and Kubler et al. (2015) J. Immunother Cancer 3:26). See also, Pardi et al. Nat Rev Drug Discov. 2018 April; 17(4): 261-279, which are incorporated by reference herein in their entirety.

In some embodiments, the method comprises subjecting the subject to radiation therapy, surgery, or a combination thereof. For example, a surgery can be open surgery or minimally invasive surgery.

In some embodiments, the subject is refractory to standard therapy (e.g., standard of care). In some embodiments, the subject has no standard therapy option. In some embodiments, the subject relapsed or progressed after standard therapy. In some embodiments, the methods provided herein are useful for treating locally advanced or metastatic solid tumors refractory to standard therapies. For example, an HPV associated cancer can be refractory to immune checkpoint inhibitors such as those described herein.

In some embodiments, the subject has a cancer that is 5 refractory or intolerant to standard therapy (e.g., administration of a chemotherapeutic agent, an immunotherapy, or radiation). In some embodiments, the subject has a cancer (e.g., a locally advanced or metastatic tumor) that is refractory or intolerant to prior therapy (e.g., administration of a chemotherapeutic agent, immunotherapy (e.g., an immune checkpoint inhibitor), or radiation). In some embodiments, the cancer that is refractory or intolerant to standard therapy is an HPV-associated cancer. In some embodiments, the subject has a cancer (e.g., a locally advanced or metastatic tumor) that has no standard therapy.

In some embodiments, the subject has undergone prior therapy. In some embodiments, the subject received treatment with a platinum-based chemotherapy, immune checkpoint inhibitor (e.g., PD-1/PDL1 immunotherapy), radiation therapy, or a combination thereof, prior to treatment with 2-S rimantadine, or pharmaceutically acceptable salt thereof.

Optimal dosages of pure 2-S rimantadine, or pure 2-R rimantadine or pharmaceutically acceptable salt thereof (e.g., a salt of 2-S rimantadine), to be administered to a subject can be determined by those skilled in the art, and will vary with the mode of administration, the strength of the preparation, the mode of administration, and the advancement of the disease condition. In some embodiments, a subject can be administered a dosage of 2-S rimantadine, or pharmaceutically acceptable salt thereof, of about 0.01 to 10,000 mg per 25 adult human per day. For example, a pharmaceutical composition comprising pure 2-S rimantadine, or pure 2-R rimantadine, or racemic rimantadine, or pharmaceutically acceptable salt thereof (e.g., a salt of 2-S rimantadine), can be formulated to provide a dosage of about 0.01, about 0.05, about 0.1, about 0.5, about 1, about 2.5, about 5, about 10, about 15, about 25, about 50, about 100, about 150, about 200, about 250 or about 500 milligrams of rimantadine, or pharmaceutically acceptable salt thereof. In some embodiments, an effective amount of pure 2-S rimantadine, pure 2-R rimantadine or pharmaceutically acceptable salt thereof, can be provided at a dosage level of about 0.1 mg/kg to about 1000 mg/kg of body weight per day, or any range therein. For example, about 0.5 to about 500 mg/kg of body weight per day, about 1.0 to about 250 mg/kg of body weight per day, about 0.1 to about 100 mg/kg of body weight per day, 0.1 to about 50.0 mg/kg of body weight per day, 15.0 mg/kg of body weight per day, or about 0.5 to about 7.5 mg/kg of body weight per day. Pure 2-S rimantadine, or pure 2-R rimantadine or pharmaceutically acceptable salt thereof, can be administered to a subject on a regimen of 1 to 5 times per day or in a single daily dose.

In one aspect, the compounds disclosed herein are used in the preparation of medicaments for the treatment of diseases or conditions described herein. In addition, a method for treating any of the diseases or conditions described herein in a subject in need of such treatment, involves administration of pharmaceutical compositions that include at least one compound disclosed herein or a pharmaceutically acceptable salt, active metabolite, prodrug, or solvate thereof, in therapeutically effective amounts to said subject.

In certain embodiments, the compositions containing the compounds disclosed herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation clinical trial.

In prophylactic applications, compositions containing the compounds disclosed herein are administered to a patient susceptible to or otherwise at risk of a particular disease, disorder or condition.

In certain embodiments, the dose of drug being administered may be temporarily reduced or temporarily suspended for a certain length of time (i.e., a “drug holiday”).

### Methods of Detection of HPV

Another aspect of the present disclosure comprises methods of treating cancer in a subject, the method comprising detecting in a sample from the subject a cancer cell that expresses a HPV protein and then administering to the subject a therapeutically effective amount of any one of the pharmaceutical compositions described herein (e.g., a pharmaceutical composition comprising a salt of 2-S rimantadine). The detection methods described herein are based on determining the presence or absence of an HPV protein or of a functionally equivalent variant thereof. In some embodiments, wherein the presence of an HPV protein or of a functionally equivalent variant thereof is detected in a sample from the subject, the expression level of the HPV protein is determined. In some embodiments, the HPV protein is HPV16 E5. In some embodiments, the pharmaceutical composition comprises at least one additional therapeutic agent selected from one or more additional anti-cancer therapies or therapeutic agents (e.g., chemotherapeutic agents).

Thus, in another aspect, the present disclosure relates to an in vitro method for the diagnosis of diseases associated the presence of an HPV protein in a subject or for determining the predisposition of a subject to suffer from said disease associated with the presence of an HPV protein, or for determining the stage or severity of said disease associated with the presence of an HPV protein in a subject, or for monitoring the effect of the therapy administered to a subject with said disease associated with the presence of an HPV protein, which comprises quantifying the expression levels of an HPV protein or of a functionally equivalent variant thereof in a biological sample from said subject, wherein an increase of the expression of the gene encoding an HPV protein or of a functionally equivalent variant thereof, with respect to the expression levels of the gene encoding an HPV protein or of a functionally equivalent variant thereof in a control sample, is indicative of a disease associated with the presence of an HPV protein, or of a greater predisposition of said subject to suffer from a disease associated with the presence of an HPV protein or of the non-response to the therapy administered to said subject. In some embodiments, the HPV protein is HPV16 E5. In some embodiments, the pharmaceutical composition comprises at least one additional therapeutic agent selected from one or more additional anti-cancer therapies or therapeutic agents (e.g., chemotherapeutic agents).

Therefore, as it is used herein the term “functionally equivalent variant” also includes any functionally equivalent fragment of said marker proteins. The term “fragment” relates to a peptide comprising a portion of said marker protein. In this case, a functionally equivalent fragment is a peptide or protein comprising a portion said marker protein and having essentially the same functions as said protein. “Marker protein” preferably refers to an HPV protein, without being limited thereto.

As will be understood by the persons skilled in the art, the detecting normally may not be correct for 100% of the subjects, although it is preferable. However, the term requires being able to identify a statistically significant part of the subjects as possessing enough quantity of the protein-of-interest such that the subject suffers from a disease associated with the presence of the protein-of-interest or has a predisposition to same. The person skilled in the art can determine if a part is statistically significant by simply using one or several well-known statistical evaluation tools, for example, determination of confidence intervals, determination of the p-value, Student's t-test, Mann-Whitney test, etc. The details are in Dowdy and Wearden, Statistics for Research, John Wiley & Sons, New York 1983. The preferred confidence intervals are at least 50%, at least 60%, at least 70%, at least 80%, at least 90%, at least 95%. The p-values are preferably 0.2, 0.1, 0.05.

As used herein, the term “predisposition” means that a subject has still not developed the disease or any of the symptoms of the disease mentioned above or other diagnostic criteria but will, however, develop the disease in the future with a certain probability. Said probability will be significantly different from the statistical probability of onset of a disease associated with the presence of an HPV protein. It is preferably diagnosed that the probability of developing a disease associated with the presence of an HPV protein is at least 30%, at least 40%, at least 50%, at least 60%, at least 70%, at least 80%, at least 90% or 100% of a predisposition. The diagnosis of a predisposition can sometimes be referred to as prognosis or prediction of the probability of a subject developing the disease.

In the context of the present disclosure, “control sample” is understood as the reference sample which is used to determine the variation of the expression levels of the genes and proteins used in the present disclosure. In an embodiment, the reference value is obtained from the provided signal using a sample of tissue obtained from a healthy individual. Preferably, samples are taken from the same tissue of several healthy individuals and combined, such that the amount of polypeptides in the sample reflects the mean value of said molecules in the population.

Thus, in a particular embodiment of the present disclosure, the expression levels of an HPV protein can be quantified. In some embodiments, the HPV protein is HPV16 E5.

As is understood by the person skilled in the art, the expression level of a protein can be quantified by means of any conventional method. By way of non-limiting illustration, the levels of protein can be quantified, for example, by means of the use of antibodies with the capacity to bind to said proteins (or to fragments thereof containing an antigenic determinant) and the subsequent quantification of the complexes formed. The antibodies which are used in these assays may or may not be labeled. Illustrative examples of markers which can be used include radioactive isotopes, enzymes, fluorophores, chemiluminescent reagents, enzyme substrates or cofactors, enzyme inhibitors, particles, dyes, etc. There is a large variety of known assays which can be used in the present disclosure which use non-labeled antibodies (primary antibody) and labeled antibodies (secondary antibody); these techniques include Western-blot, ELISA (enzyme-linked immunosorbent assay), RIA (radioimmunoassay), competitive EIA (competitive enzyme immunoassay), DAS-ELISA (double-antibody sandwich ELISA), immunocytochemical and immunohistochemical techniques, techniques based on the use of biochips or microarrays of proteins which include specific antibodies or assays based on colloidal precipitation in formats such as dipsticks. In another particular embodiment, the quantification of the levels of protein is performed by means of an immoanalytical method, such as Western blot, immunohistochemistry or ELISA. In some embodiments, said immunoanalytical method comprises the antibody specific to HPV16 E5.

Likewise, the detection method of the present disclosure can be applied to any of the diseases associated with the presence of an HPV protein defined above. In a preferred embodiment, the disease associated with the presence of an HPV protein is a cancer, preferably a cancer having high levels of an HPV protein. In some embodiments, the HPV protein is HPV16 E5.

Putting the method of the present disclosure into practice comprises obtaining a biological sample from the subject to be studied. Illustrative non-limiting examples of said samples include different types of biological fluids, such as blood, serum, plasma, cerebrospinal fluid, peritoneal fluid, feces, urine and saliva, as well as samples of tissues. The samples of biological fluids can be obtained by any conventional method like the samples of tissues; by way of illustration said samples of tissues can be samples of biopsies obtained by surgical resection.

In another aspect, the present disclosure relates to a kit comprising reagents for the quantification of the expression levels of an HPV protein or of a functionally equivalent variant thereof, for the diagnosis of cancer in a subject or for determining the predisposition of a subject to suffer from said cancer, or for determining the stage or severity of said cancer in a subject, or for monitoring the effect of the therapy administered to a subject with said cancer, in which if the reagents detect an increase in the expression of said gene or said protein or functionally equivalent variant thereof with respect to a control sample, then said subject can suffer from a disease associated with the presence of an HPV protein, or present a greater predisposition to suffer from said disease associated with the presence of an HPV protein, or present a greater severity of said disease, or the administered therapy is not being effective. In some embodiments, the HPV protein is HPV16 E5. In some embodiments, the pharmaceutical composition comprises at least one additional therapeutic agent selected from one or more additional anti-cancer therapies or therapeutic agents (e.g., chemotherapeutic agents).

The present disclosure also relates to the use of said kit.

All the terms and expressions used in the definition of the use of the kit have been described above and explained for other inventive aspects and particular embodiments of the present disclosure, and are also applicable to the use of the kit described herein.

Methods for Designing Customized Therapies and for Selecting Patients who can Benefit from Administration of 2-S Rimantadine (e.g., a salt of 2-S rimantadine) or 2-R Rimantadine

In another aspect, the present disclosure relates to an in vitro method for designing a customized therapy for a patient suffering from a disease associated with the presence of an HPV protein comprising:
(a) quantifying the expression levels of an HPV protein in said patient, and
(b) comparing said expression levels with control levels,
wherein if the expression levels of an HPV protein in said patient are greater than the control values, then a therapeutically effective amount of 2-S rimantadine or a pharmaceutically acceptable salt thereof, or 2-R rimantadine or pharmaceutically acceptable salt thereof (e.g., a salt of 2-S rimantadine), is administered to said patient.

In some embodiments, the HPV protein is HPV16 E5. In some embodiments, at least one additional therapeutic agent selected from one or more additional anti-cancer therapies or therapeutic agents (e.g., chemotherapeutic agents) is administered to the patient.

In another aspect, the present disclosure relates to an in vitro method for selecting patients suffering from a disease associated with the presence of an HPV protein, to be treated with a therapeutically effective amount of 2-S rimantadine or a pharmaceutically acceptable salt thereof, or 2-R rimantadine or pharmaceutically acceptable salt (e.g., a salt of 2-S rimantadine) thereof comprising
a) quantifying the expression levels of an HPV protein in said patient, and
b) comparing said expression levels with control levels, wherein if the expression levels of an HPV protein in said patient are greater than the control values, then said patient is selected to receive a therapeutically effective amount of 2-S rimantadine or a pharmaceutically acceptable salt thereof (e.g., a salt of 2-S rimantadine), or 2-R rimantadine or pharmaceutically acceptable salt thereof.

In some embodiments, the HPV protein is HPV16 E5. In some embodiments, at least one additional therapeutic agent selected from one or more additional anti-cancer therapies or therapeutic agents (e.g., chemotherapeutic agents) is administered to the patient.

For further description of the components and methods disclosed herein, see, for example, U.S. Prov. App. No. 63/150,027, which is incorporated herein by reference in its entirety.

### Examples

The present disclosure is further illustrated by the following examples, which should not be construed as limiting in any way. The experimental procedures to generate the data shown are discussed in more detail below. The disclosure has been described in an illustrative manner, and it is to be understood that the terminology used is intended to be in the nature of description rather than of limitation.

### General Experimental, Instrument, and Methodology Details

### X-Ray Powder Diffraction (XRPD)

For XRPD analysis, a Malvern-Panalytical Aeris X-ray powder diffractometer was used. The XRPD parameters used are as follows; All samples were placed directly on the sample holder without external force. The detector used is a PIXcel detector. The scan mode used is continuous scan. The radiation source is Cu (ka). No monochromator was used. The X-ray generator power was 45 kV, 15 mA and the Goniometer diameter was 290 mm. The step size was 0.0109 and time per step was 0.0795 seconds. The scan range was 3-40. The slits were a fixed Fixed 1/8° Divergence slit. The sample was measured on a zero background holder and calibrated using a Panalytical Si reference standard disc.

Differential Scanning calorimetry (DSC) and Thermo-Gravimetric Analysis (TGA)

TGA data were collected using a TA Discovery 550 using a Nickel calibration standard and platinum sample pan. Upon measurement of mass, samples were placed in the measurement chamber and equilibrated in a stream of nitrogen gas.

DSC data were collected using a TA 2500 DSC instrument and calibrated using an indium standard. Samples were weighed, purged with nitrogen, and underwent heating while analyzed for mass change.

### Example-1. ScreenPatch® Assay on NR1/NR2A and NR1/NR2B

### ScreenPatch

NR1/NR2A ionotropic receptor encoded by the human GRIN1/GRIN2A genes, expressed in HEK293 cells.
NR1/NR2B ionotropic receptor encoded by the human GRIN1/GRIN2B genes, expressed in HEK293 cells

### Formulation

2-S rimantadine, 2-R rimantadine, racemic rimantadine and amantadine solutions were prepared daily and prepared by diluting stock solutions into an appropriate HEPES-buffered physiological saline (HB-PS) solution. Because 0.6% DMSO does not affect channel current, all test and control solutions contained 0.6% DMSO. Test article formulations were sonicated (Model 2510/5510, Branson Ultrasonics, Danbury, CT), at room temperature for at least 20 minutes to facilitate dissolution.

Test article effects were evaluated in 8-point concentration-response format (4 replicate wells/concentration). All test and control solutions contained 0.6% DMSO. The test article formulations were loaded in a 384-well compound plate using an automated liquid handling system (Assist Plus, INTEGRA).

### Positive Control Treatment Groups

Stock solutions of positive control articles were prepared in batches, aliquoted for individual use, stored frozen, and were used within six months. The positive control test solutions were prepared fresh daily. The final DMSO concentration was 0.6% (v/v). The NMDA receptors agonists, L-glutamate and glycine were used as a reference agonist in this study. The NMDA receptors antagonist, amantadine, was used as a reference antagonist in this study.

### Testing and Concentrations:

Test articles were evaluated for functional effects on ion channels. Test concentrations are shown in Table 1 below.

TABLE 1
Test Articles Concentrations

| | | | Purity | Volume (μL) | |
|---|---|---|---|---|---|
| # | Test Article ID | MW | (%) | 100 mM stock | Test concentrations, μM |
| 1 | racemic rimantadine | 215.77 | 99.9 | 340 | 0.2, 0.6, 2, 6, 20, 60, 200, 600 |
| 2 | amantadine | 187.72 | 99.9 | 340 | 0.2, 0.6, 2, 6, 20, 60, 200, 600 |
| 3 | 2-R rimantadine | 215.77 | 95 | 285 | 0.2, 0.6, 2, 6, 20, 60, 200, 600 |
| 4 | 2-S rimantadine | 215.77 | 98 | 295 | 0.2, 0.6, 2, 6, 20, 60, 200, 600 |

### Cloned Test Systems

Cells were maintained in tissue culture incubators. Stocks were maintained in cryogenic storage. Cells used for electrophysiology were plated in plastic culture dishes.

### HEK293 Culture Procedures

HEK293 cells were transfected with the appropriate ion channel or receptor cDNA(s) encoding NR1 and NR2A-B. Stable transfectants were selected using the G418 and Zeocin-resistance genes incorporated into the expression plasmid. Selection pressure was maintained with 0418 and Zeocin in the culture medium. Cells were cultured in Dulbecco's Modified Eagle Medium/Nutrient Mixture F-12 (D-MEM/F-12) supplemented with 10% fetal bovine serum, 100 U/mL penicillin O sodium, 100 μg/mL streptomycin sulfate, 100 μg/mL Zeocin, 5 μg/mL blasticidin and 500 μg/mL 0418.

### ScreenPatch Test Methods:

All experiments were performed at ambient temperature. Target-specific test procedures are described below. The following procedures apply to all ScreenPatch assays.

Before testing, cells in culture dishes were washed twice with HBSS solution. Immediately before use in IonWorks Barracuda™, the cells were washed in HB-PS containing 6 mM CaCh to improve sealing.

Test articles were evaluated in 8-point concentration-response format (4 replicate wells/concentration, see Table 1). Previous results have shown that 0.6% DMSO does not affect channels currents; thus, unless specified otherwise, all test and control solutions contained 0.6% DMSO. The test article formulations were loaded in a 384-well compound plate and placed in the IonWorks Barracuda™ plate well.

Positive control articles were prepared in batches, aliquoted for individual use, stored frozen, and used within six months. The positive control test solutions were prepared fresh daily. The final DMSO concentration was 0.6%.

2× concentration of test article (as specified in Table 2) was pre-applied 2 minutes before application of L-glutamate (Sigma-Aldrich)/glycine (Sigma-Aldrich (SuM L-glutamate and 50 μM glycine) mixed with 1× concentration of test article.

To monitor the sensitivity of the assay, the antagonist positive control, amantadine hydrocholoride (Sigma-Aldrich) was applied at 8 half log concentrations (range 0.3-1000 M); n=4, where n=the number of replicates per concentration. The agonist positive control (L-glutamate) was applied at eight (8) concentrations (0.03-100 μM; n=4, where n=the number of replicates) together with 50 μM glycine.

Inhibitory effects of compounds on the channels were calculated as:

$$Response = Base + \frac{Max - Base}{1 + {\{ \frac{xhalf}{x} \}}^{rate}}$$

where Base is the response at low concentrations of test article, Max is the maximum response at high concentrations, xhalf is the EC₅₀, or IC₅₀, the concentration of test article producing either half-maximal activation or inhibition, and rate is the Hill coefficient. Nonlinear least squares fits were made assuming a simple binding model. If appropriate, fits were weighted by the standard deviation. No assumptions about the fit parameters were made; the fit parameters were determined by the algorithm.

Nonlinear least squares fits were solved with the XLfit add-in for Excel 2016 (Boston, MA).

Effects of the compounds were evaluated with two types of measurements:
1 Peak current amplitude (PCA) measurements at current maximum.
2. Steady state current amplitude (SSC) measurements, as mean between 4 and 5 seconds following stimulation of receptors with agonist.

### Procedures

### Electrophysiological Procedures:

a) Intracellular solution (mM): 50 mM CsCl, 90 mM CsF, 2 mM MgCl₂, 5 mM EGTA, 10 mM HEPES. Adjusted to pH 7.2 with CsOH. This solution was prepared in batches and stored refrigerated. In preparation for a recording session, the intracellular solution was loaded into the intracellular compartment of the PPC planar electrode.
b) Extracellular solution, HB-PS (composition in mM): NaCl, 137; KCl, 1.0; CaCl₂), 5; HEPES, 10; Glucose, 10; pH adjusted to 7.4 with NaOH (refrigerated until use).
c) Holding potential: −70 mV, potential during test articles application: −70 mV.

### Recording Procedure:

a) Extracellular buffer was loaded into the PPC plate wells (11 μL per well). Cell suspension was pipetted into the wells (9 μL per well) of the PPC planar electrode.
b) Whole-cell recording configuration was established via patch perforation with membrane currents recorded by on-board patch clamp amplifiers.
c) Two recordings (scans) were performed. First scan, during test articles addition at 2× concentration to detect potential agonist effect and for preincubation of test articles with cells (for 2 minutes). Second, during agonist stimulation of receptors (5 μML-glutamate and 50 μM glycine) co-applied with 1× concentration of test articles to detect antagonist effects of the test articles.

Test article administration: The application consisted of the addition of 20 μL of 1× concentrated test article solution and agonist at 10 μL/s (2 second total application time).

Positive control agonist: 0.03-100 AM L-glutamate (8 concentration dose-response, half log scale) and 50 μM glycine

Positive control antagonist: 0.3-1000 AM amantadine (8 concentration-response, half log scale) co-applied with 5 μM glutamate and 50 μM glycine.

Evaluation of effects were based on peak current measurements.

### Results

Agonist and antagonist properties of four (4) compounds were examined using an HTS electrophysiology-based approach, Ion Work Barracuda™ (IWB). A two-application protocol was employed.

Agonist Format: The potential agonist effect of the test articles and the positive control antagonist, amantadine, were examined during first application. Neither test articles nor amantadine had produced significant activation of NMDA receptors (data not shown)

Antagonist Format: The antagonist activity of test articles was examined during second application of compounds after stimulation of receptors with 5 μM L-glutamate and 50 μM glycine. It was found that all four test articles produced significant concentration dependent inhibition of NMDA receptors function. To access open channel block type of inhibition, peak and steady state current amplitudes (between 4^{th} and 5^{th} seconds after agonist application) were measured (PCA and SSC respectively). Table 2 shows an average of compounds' IC₅₀ at NR1/NR2A and NR1/NR2B receptors with these two types of measurements.

Amantadine produced inhibition of NR1/NR2A receptors with IC_{50_PCA}=97.8 μM and IC_{50_SSC}=48.9 μM for peak and steady state currents amplitudes respectively. NR1/NR2B receptors were inhibited by amantadine with IC_{50_PCA}=22.0 μM and IC_{50_SSC}=17.9 μM for peak and steady state currents amplitudes respectively. Leftward shift in amantadine potency for steady state currents measurements suggests, at least in part, open channel block mechanism of inhibition for NR1/NR2A NMDA receptors.

TABLE 2
Summary of inhibition IC₅₀ produced by test articles
and reference antagonist, amantadine.

| | | Peak Current | | Steady State Current | |
|---|---|---|---|---|---|
| | | NR2A | NR2B | NR2A | NR2B |
| # | Compounds ID | IC₅₀, μM | IC₅₀, μM | IC₅₀, μM | IC₅₀, μM |
| 1 | racemic rimantadine | 54.02 | 32.20 | 40.14 | 30.39 |
| 3 | 2-S rimantadine | 57.16 | 34.82 | 33.89 | 35.39 |
| 4 | 2-R rimantadine | 61.83 | 23.61 | 38.31 | 19.64 |
| 5 | Amantadine | 97.81 | 14.25 | 46.76 | 11.55 |
| 6 | Glutamate (EC₅₀) | 3.18 | 3.70 | 1.20 | 0.71 |

Amantadine produced inhibition of NR1/NR2A receptors with IC_{50_PCA}=97.8 μM and IC_{50_SSC}=48.9 μM for peak and steady state currents amplitudes respectively. NR1/NR2B receptors were inhibited by amantadine with IC_{50_PCA}=22.0 μM and IC_{50_SSC}=17.9 μM for peak and steady state currents amplitudes respectively. Leftward shift in amantadine potency for steady state currents measurements suggests, at least in part, open channel block mechanism of inhibition for NR1/NR2A NMDA receptors.

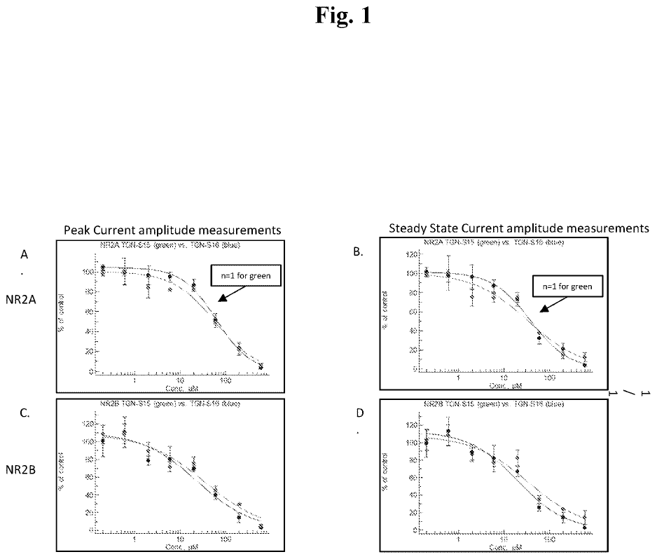

The results of these assays are further exemplified in FIG. 1A-D.

### Example 2. Effect of Pure 2-S Rimantadine or Pure 2-R Rimantadine in Mouse Cancer Models

### Methods

### Cell Lines

AT-84-E7 and B16-OVA are grown in RPMI 1640 containing 10% FBS, 1% L-flutamine, 1% penicillin/streptomycin, 1% sodium pyruvate, and 200 μg/ml G418. DC2.4, RAW264.7, B3Z, 4T1, B16 and MC38 are grown in RPMI 1640 20 containing 10% FBS, 1% L-glutamine, 1% penicillin/streptomycin, and 1% sodium pyruvate. HEK293T is grown in DMEM containing 10% FBS, 1% L-glutamine, and 1% penicillin/streptomycin. 4MOSC1 is cultured in collagen-coated dish with KSFM media (Invitrogen, Carlsbad, 20 CA) supplemented 1% penicillin/streptomycin, 5 ng/ml EGF (Invitrogen), and 2×10⁻¹¹ M cholera toxin (Sigma, St. Louis, MO) (27). CAL-27, CAL-33, and SCC-47 are grown in DMEM containing 10% FBS, 22 1% L-glutamine, and 1% penicillin/streptomycin. Routine monitoring for Mycoplasma contamination is performed using the MycoAlert PLUS Detection Kit (Lonza, Basel, Switzerland). All cell lines are used within ten passages after thawing.

### Mouse Studies

Mice are injected subcutaneously with 1.0 to 5.0×10⁵ AT-84-E7, 1.5×10⁵ B16-OVA, or 5.0×10^{5 4}T1 cells are resuspended in 100 μl of PBS in the right flank. For orthotopic models, 1.0×10⁵ AT-84-E7 or 1.0×10^{6 4}MOSC1 in 30 μl of PBS are injected into tongue. Tumor diameter is measured every 2 to 3 days with an electronic caliper and reported as volume using the formula; tumor volume (mm³)=(length×width²)/2. Once tumors are palpable, mice are treated with 200 μg of anti-PDL1 antibody (BioXcell, West Lebanon, NH) via IP injection every 3 days for a total of three or four injections per mouse, or 5 mice are treated with 10-20 mg/kg body weight of pure 2-S rimantadine or pure 2-R rimantadine via IP injection daily for 7 days. For adoptive transfer experiments, first, single-cell suspension of spleen from OT-1 mice are cultured in media containing 10 ng/ml OVA SIINFEKL peptide (InvivoGen, San Diego, CA) and 2 ng/ml recombinant IL-2 (PeproTech, Rocky Hill, NJ) for days, and then 4.0×10⁶ cells are intravenously injected into B16-OVA-bearing mice.

### Flow Cytometry

Single-cell suspensions are prepared from, lung, liver, tumor-draining lymph nodes, and tumors by mechanical dissociation and are filtered using a 70 μm filter. AT-84-E7 and MOC2 tumors are incubated in collagenase D (Roche, Basel, Switzerland) at 37 °C for 1 hour prior to mechanical dissociation. Density gradient centrifugation on 40%/80% Percoll (GE Healthcare, Chicago, IL) gradient is performed for single-cell suspension from tumors. After obtaining single-cell suspensions, each sample is incubated with an Fc blocking reagent (anti-CD16/32 antibody; BioLegend, San Diego, CA). Following Fc blockade, cells are stained with fluorescent-labeled antibodies [BioLegend, BD Bioscience (San Jose, CA), or eBiosciences (Thermo Fisher Scientific, Waltham, MA)]. LIVE/DEAD Fixable Cell Staining Kit (Invitrogen) is used for viability staining. For intracellular staining, cells were processed with Foxp3/Transcription Factor Fixation/Permeabilization Concentrate and Diluent (Invitrogen). Cells are analyzed using a BD FACS Aria II or LSR II flow cytometer (BD). Data is analyzed on FlowJo (FlowJo, LLC, Ashland, OR). For each antibody, the following clones are used: CD45.2 (104), CD3e (145-2C11), CD4 (RM4-5), CD8a (5H10), CD25 (3C7, PC61), CD44 (IM7), CD62L (MEL-14), IFN-γ (XMG1.2), Foxp3 (MF23), H-2Kb (AF6-88.5), H-2Kk (36-7-5), H-2Kd (SF1-1.1), H-2Kb/SIINFEKL (eBio25-D1.16), I-A/I-E (2G9), CD49b (DX5), CD11b (M1/70), FLAG (L5), CD31 (MEC13.3), NK-T/NK Cell Antigen (U5A2-13), CD102 (3C4 (MIC2/4)), CD62P (RMP-1), CD105 (MJ7/18), CD106 (429 (MVCAM.A)), and CD162 (2PH1). H-2Kb/SIINFEKL tetramer was purchased from MBL International (Woburn, MA).

### Cell Cycle and Proliferation Assays

Cell cycle progression was analyzed on the basis of BrdU incorporation following 1 cell staining with BrdU-APC and 7-AAD using BD Pharmingen BrdU Flow Kit. (BD, Franklin Lakes, NJ) according to the manufacture's protocol. Cells are analyzed using flow cytometry. Cell proliferation is assessed by using MTT [3-(4,5-dimethylthiazol-2-yl)-2,5-diphenyltetrazolium bromide]. First, cells are seeded in 96-well plate and cultured for 2-3 days. Next, culture media is replaced with fresh media containing 0.5 mg/ml of MTT (Sigma) and the plates are incubated for 4 hours at 37 °C. Then, purple formazan crystals are dissolved in lysis buffer (4 mM HCl and 0.1% NP-40 in isopropanol) and the absorbance is recorded on a TECAN infiniteM200 microplate reader (Tecan, Männedorf, Switzerland) at a wavelength of 570 nm with absorbance at 650 nm as reference.

### B3Z Activation Assay

B16-OVA cells are seeded into a 96-well plate and treated with 100 μM pure 2-R rimantadine, pure 2-R rimantadine or racemic rimantadine for 24 hours, prior to addition of B3Z cells. After 24 hours of co-culture, medium is removed and 100 μl of lysis buffer [0.155 mM chlorophenol red β-D-galactopyranoside (CPRG) (Roche), 0.125% Nonidet P-40 Alternative (EMDCalbiochem), and 9 mM MgCl₂ (Sigma) in PBS] are added. After incubation for 4 hours at 37 °C, the absorbance at 570 nm is determined on a TECAN infinite M200 microplate reader.

### Reverse Transcription and Quantitative PCR

Total RNA is extracted using TRIzol Reagent (Invitrogen) and reverse transcribed with qScript cDNA Synthesis Kit (Quanta BioSciences, Beverly, MA) according to the manufacturer's instructions. Quantitative PCR analysis is conducted by using KAPA SYBR 1 FAST (KAPA Biosystems, Wilmington, MA) on the 7900HT Fast Real-Time PCR System (Applied Biosystems, Foster City, CA).

### Results

Pure 2-S Rimantadine and pure 2-R rimantadine have anti-tumor activity alone and significantly decreased tumor growth. Six mice are inoculated with 5×10⁵ AT84-E7/E5 tumor cells and treated with intraperitoneal (IP) injections of 10 mg/kg body weight pure 2-S rimantadine or pure 2-R rimantadine once daily for a total of 7 injections starting on day 8. The tumor volumes are measured over the course of the experiment. Mice that receive pure 2-S rimantadine or pure 2-R rimantadine show statistically significant decreases in tumor size compared to control groups. Six mice are inoculated with 1.5×10⁵ B16-OVA tumor cells and treated with IP injections of 10 mg/kg body weight pure 2-S rimantadine or pure 2-R rimantadine once daily for a total of 7 injections starting on day 10. The tumor volumes are measured over the course of the experiment. Mice that receive pure 2-S rimantadine or pure 2-R rimantadine show statistically significant decreases in tumor sizes compared to control groups. This experiment is repeated three times with similar results. Five mice are inoculated with 5×10^{5 4}T1 tumor cells and treated with IP injections of 10 mg/kg body weight pure 2-S rimantadine or pure 2-R rimantadine once daily for a total of 7 injections starting on day 6. The tumor volumes are measured over the course of the experiment. Mice that receive 2-S rimantadine or pure 2-R rimantadine show statistically significant decreases in tumor sizes compared to control groups. The anti-tumor effect of pure 2-S rimantadine or pure 2-R rimantadine is decreased in AT-84-E7 tumors which do not express E5. Significant increases in surface expression of MHC is observed in multiple cell lines. Cell surface expression of MHC I on E5-positive AT-84-E7 is restored with pure 2-S rimantadine or pure 2-R rimantadine treatment.

To test the ability of 2-S rimantadine to enhance functional antigen presentation on tumor cells, B16 cells expressing OVA are used as a model tumor antigen and coculture with B3Z cells which respond to OVA SINNFKL peptide. Treatment of B16-OVA cells with pure 2-S rimantadine or pure 2-R rimantadine results in a significant 3-fold increase in recognition of this model tumor antigen by B3Z cells. Pure 2-S rimantadine or pure 2-R rimantadine with anti-PDL1 immunotherapy results in a significant improvement in survival in mice harboring B16-OVA tumors.

The ability for pure 2-S rimantadine or pure 2-R rimantadine to increase expression of MHC on antigen presenting cells using the RAW264.7 cell line is tested and significant increases in both MHC class I and MHC class II surface expression is observed. These findings demonstrate that pure 2-S rimantadine or pure 2-R rimantadine has novel anti-tumor activity in multiple pre-clinical tumor models and functions to enhance antigen presentation by upregulating MHC.

To study the direct cytotoxic activity of pure 2-S rimantadine or pure 2-R rimantadine, in vitro BrdU incorporation assays are performed to quantify the effects of pure 2-S rimantadine or pure 2-R rimantadine on cell cycling in human HNSCC cell lines. Pure 2-S rimantadine or pure 2-R rimantadine alone result in significant increases in G0/G1 cell cycle arrest and significant decreases in S phase in both AT-84-E7 and B16-OVA models. Suppression of cell proliferation is also observed. Analysis of the effect of pure 2-S rimantadine or pure 2-R rimantadine on proliferation of T cells is tested, but there are no significant effects.

Changes in gene expression of cell cycle proteins caused by pure 2-S rimantadine or pure 2-R rimantadine are screened using RTqPCR and significant decreases in microtubule and cell cycle regulatory molecule Stathmin is seen. Decreases in microtubule associated molecule Tau is also observed.

To confirm pure 2-S rimantadine or pure 2-R rimantadine has activity against human head and neck tumor lines, BrdU incorporation assays and proliferation assays are performed. Significant cell cycle arrest and decreased proliferation with rimantadine alone is observed in the human CAL-27, CAL-33, and SCC-47 squamous cell carcinoma cell lines. Finally, pure 2-S rimantadine or pure 2-R rimantadine induce cell cycle arrest in murine and human cell lines engineered to express HPV16 E5, indicating that pure 2-S rimantadine or pure 2-R rimantadine is able to functionally reverse effects of HPV E5.

### Example 3. HPV Genotyping

HPV genotyping is known in the art, for example, see Sichero et al., 2017, Cancer Epidemiol Biomarkers, 26 (8): 1312-132θ. For example, DNA is extracted from exfoliated cervical cells by spin-column chromatography. Mucosal alpha-HPVs are tested using PCR amplification with primers such as MY09/11 and PGMY09/11 (see Table 3) followed by genotyping via hybridization with HPV type-specific oligonucleotide probes and restriction fragment length polymorphism analysis. Negative and positive controls are used to ascertain the quality of template DNA.

TABLE 3
primer sequences

| | Sequence | |
|---|---|---|
| | Name | Sequence (5′-3′) |
| PGMY09/ | PGMY11-A | GCA CAG GGA CAT AAC AAT GG |
| 11 | | SEQ ID NO: 1 |
| | PGMY11-B | GCG CAG GGC CAC AAT AAT GG |
| | | SEQ ID NO: 2 |
| | PGMY11-C | GCA CAG GGA CAT AAT AAT GG |
| | | SEQ ID NO: 3 |
| | PGMY11-D | GCC CAG GGC CAC AAC AAT GG |
| | | SEQ ID NO: 4 |
| | PGMY11-E | GCT CAG GGT TTA AAC AAT GG |
| | | SEQ ID NO: 5 |
| | PGMY09-F | CGT CCC AAA GGA AAC TGA TC |
| | | SEQ ID NO: 6 |
| | PGMY09-G | CGA CCT AAA GGA AAC TGA TC |
| | | SEQ ID NO: 7 |
| | PGMY09-H | CGT CCA AAA GGA AAC TGA TC |
| | | SEQ ID NO: 8 |
| | PGMY09-I | G CCA AGG GGA AAC TGA TC |
| | | SEQ ID NO: 9 |
| | PGMY09-J | CGT CCC AAA GGA TAC TGA TC |
| | | SEQ ID NO: 10 |
| | PGMY09-K | CGT CCA AGG GGA TAC TGA TC |
| | | SEQ ID NO: 11 |
| | PGMY09-L | CGA CCT AAA GGG AAT TGA TC |
| | | SEQ ID NO: 12 |
| | PGMY09-M | CGA CCT AGT GGA AAT TGA TC |
| | | SEQ ID NO: 13 |
| | PGMY09-N | CGA CCA AGG GGA TAT TGA TC |
| | | SEQ ID NO: 14 |
| | PGMY09-P | G CCC AAC GGA AAC TGA TC |
| | | SEQ ID NO: 15 |
| | PGMY09-Q | CGA CCC AAG GGA AAC TGG TC |
| | | SEQ ID NO: 16 |
| | PGMY09-R | CGT CCT AAA GGA AAC TGG TC |
| | | SEQ ID NO: 17 |
| | HMB01 | GCG ACC CAA TGC AAA TTG GT |
| | | SEQ ID NO: 18 |
| | MY09 | CGT CCM ARR GGA WAC TGA TC |
| | | SEQ ID NO: 19 |
| | MY11 | GCM CAG GGW CAT AAY AAT GG |
| | | SEQ ID NO: 20 |

The degenerate base code is as follows: M = A or C, W = A or T, Y = C or T, and R = A or G.

### Example 4. 2-S Rimantadine and 2-R Rimantadine Toxicology in Vivo

A series of in-vivo experiments are performed to determine whether 2-S rimantadine or 2-R rimantadine have higher binding selectivity for any one of glutamate, GABA, dopamine receptors, or any combination thereof. Enhanced selectivity of any one of glutamate, GABA, dopamine receptors, or any combination thereof by 2-S rimantadine or 2-R rimantadine compared to racemic rimantadine results in the absence of central nervous system adverse effects including nausea, upset stomach, vomiting, anorexia, dry mouth, abdominal pain, asthenia, nervousness, tiredness, lightheadedness, dizziness, headache, trouble sleeping, difficulty concentrating, confusion and anxiety, commonly associated with racemic rimantadine.

To test the above, mice are treated with 10-20 mg/kg body weight of pure 2-S rimantadine, pure 2-R rimantadine, racemic rimantadine (control), or amantadine (control) via IP injection daily for 7 days. Next, a series of SPECT analyses as described in Schramm, N., et al. (2000). Compact high resolution detector for small animal SPECT, are conducted for each of glutamate, GABA, dopamine receptors to assess the binding selectivity of 2-S rimantadine and 2-R rimantadine.

The SPECT analyses comprise of treatment of the mice with radioligands specific to each receptor. For example, IBZM has been documented to have a high affinity for the D2/3 dopamine receptor. Radioligands specific to glutamate and GABA receptors are known to those skilled in the art. The appropriate amount of the respective radioligands for each of glutamate, GABA, dopamine receptors are injected into the lateral tail vein of the mice and SPECT measurements commence 45 mins after radioligand administration.

Surprisingly, 2-R rimantadine has significantly higher binding selectivity or agonistic behavior to glutamate, GABA, dopamine receptors or pathways, or any combination thereof as compared to 2-S rimantadine. Thus, 2-R rimantadine results in a higher incidence of central nervous system adverse effects including nausea, upset stomach, vomiting, anorexia, dry mouth, abdominal pain, asthenia, nervousness, tiredness, lightheadedness, dizziness, headache, trouble sleeping, difficulty concentrating, confusion and anxiety, as compared to 2-S rimantadine. Taken together with Example 2, 2-S rimantadine is significantly less toxic, while still effective, as a treatment for cancer as compared to 2-R rimantadine.

### Example 5. 2-S Rimantadine and 2-R Rimantadine Toxicology in Vivo

A series of in-vivo experiments are performed to determine whether 2-S rimantadine or 2-R rimantadine have higher binding selectivity for any one of glutamate, GABA, dopamine receptors, or any combination thereof. Enhanced selectivity of any one of glutamate, GABA, dopamine receptors, or any combination thereof by 2-S rimantadine or 2-R rimantadine compared to racemic rimantadine results in the absence of central nervous system adverse effects including nausea, upset stomach, vomiting, anorexia, dry mouth, abdominal pain, asthenia, nervousness, tiredness, lightheadedness, dizziness, headache, trouble sleeping, difficulty concentrating, confusion and anxiety, commonly associated with racemic rimantadine.

To test the above, mice are treated with 10-20 mg/kg body weight of pure 2-S rimantadine, pure 2-R rimantadine, racemic rimantadine (control), or amantadine (control) via IP injection daily for 7 days. Next, a series of SPECT analyses as described in Schramm, N., et al. (2000). Compact high-resolution detector for small animal SPECT, are conducted for each of glutamate, GABA, dopamine receptors to assess the binding selectivity of 2-S rimantadine and 2-R rimantadine.

The SPECT analyses comprise of treatment of the mice with radioligands specific to each receptor. For example, [¹²³I] IBZM has been documented to have a high affinity for the D2/3 dopamine receptor. Radioligands specific to glutamate and GABA receptors are known to those skilled in the art. The appropriate amount of the respective radioligands for each of glutamate, GABA, dopamine receptors are injected into the lateral tail vein of the mice and SPECT measurements commence 45 mins after radioligand administration.

Surprisingly, 2-S rimantadine has significantly higher binding selectivity or agonistic behavior to glutamate, GABA, dopamine receptors or pathways, or any combination thereof as compared to 2-R rimantadine. Thus, 2-S rimantadine results in a higher incidence of central nervous system adverse effects including nausea, upset stomach, vomiting, anorexia, dry mouth, abdominal pain, asthenia, nervousness, tiredness, lightheadedness, dizziness, headache, trouble sleeping, difficulty concentrating, confusion and anxiety, as compared to 2-R rimantadine. Taken together with Example 2, 2-S rimantadine is significantly less toxic, while still effective, as a treatment for cancer as compared to 2-R rimantadine.

### Example 6. 2-S Rimantadine and 2-R Rimantadine Proliferation in Vitro

Experiments were performed to determine the ability of 2-S rimantadine (also referred to as “S-rimantadine” throughout the application), 2-R rimantadine (also referred to as “R-rimantadine” throughout the application), racemic (RS) rimantadine, and memantine to effect proliferation in CAL-27 cells. S-rimantadine results in enhanced or equivalent cancer cell proliferation as compared to R-rimantadine or racemic rimantadine.

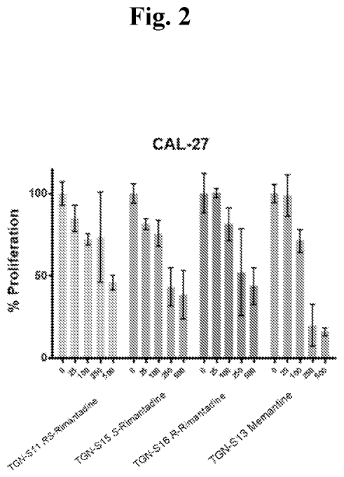

On day 1, CAL-27 cells were seeded in a 96-well plate (2-4×10³ cells/well, medium 100 μl/well) and left overnight to allow the cells to attach to the plate. On day 2, varying concentrations of rimantadine (0 μM, 100 μM, 250 μM, or 500 μM) were added to the cells and allowed to incubate for 24 hours or 48 hours. On either day 3 or 4, the culture media was aspirated, and 100 μl/well of MTT solution (comprising an MTT concentration of 0.5 mg/ml; formed by dilution of thiazolyl blue tetrazolium bromide solution (SIGMA, Cat #M2128) with stock solution (5 mg/ml in PBS (−20 °C)) was added to the culture media. The cells were incubated in a CO₂ incubator at 37 °C for 3 hours, and the MTT solution was aspirated. 100 μl/well of DMSO was then added and the cells were incubated for about 5 minutes. A OD570 nm (Ref 650 nm) was then read. Results of the experiment are shown in FIG. 2.

### Example 7. In-Vivo Tumor Model/Anti-Tumor Activity Methods

The activity of 2-S rimantadine, 2-R rimantadine, and racemic rimantadine will be tested against HPV associated tumors using in-vivo murine syngeneic tumor models. S-rimantadine will demonstrate equivalent or increased anti-tumor activity as compared to racemic rimantadine and/or R-rimantadine.

### Plasmid Construction and HPV16 E5-Expressing Stable Cell Line

Codon-optimized HPV16 E5 will be amplified. Either C-terminal or N-terminal FLAG-tagged full-length HPV16 E5 and deletion mutants will be cloned into MIP (MSCV-IRES-Puro) or pMSCV-Blasticidin vectors. All the constructs will be confirmed by DNA sequencing. For establishing HPV16 E5-expressing cell line, HEK293T cells will be cotransfected with MIP-HPV16 E5 and Ecopac (pIK6.1MCV.ecopac.UTd) using PEI reagent (Sigma-Aldrich). Retroviruses from the culture medium of these cells will then be used to infect AT-84-E7, MOC2, and CAL-27 cells, and the infected cells will be selected by puromycin. pMSCVBlasticidin-HPV16 E5 will be used for MEER cells.

### Mouse Studies

Female 6- to 8-week-old mice will be used for experiments. C3H/HeN mice and C57BL/6 and BALB/c will be used. Mice will be injected subcutaneously with 1.0 to 5.0×10⁵ AT-84-E7, 1.5×10⁵ B16-OVA, 5.0×10^{5 4}T1, or 1.0×10⁵ MOC2 cells resuspended in 100 mL of PBS in the right flank. For orthotopic models, 1.0 105 AT-84-E7 or 1.0 106 4MOSC1 in 30 mL of PBS will be injected into tongue. Once tumors become palpable, mice will be treated with 200 mg of anti-PD-L1 antibody (Bio X Cell) via i.p. injection every 3 days for a total of three or four injections per mouse, or mice will be treated with 10 mg/kg body weight of R-rimantadine, S-rimantadine, and/or racemic rimantadine via i.p. injection daily for 7 days. For adoptive transfer experiments, single-cell suspension of spleen from OT-1 mice will be cultured in media containing 10 ng/mL OVA SIINFEKL peptide (InvivoGen) and 2 ng/ml recombinant IL2 (PeproTech) for 5 days, and then 4.0 10⁶ cells will be intravenously injected into B16-OVA-bearing mice. Tumor diameter will be measured every 2 to 3 days with an electronic caliper and reported as volume using the formula; tumor volume (mm³) (length width²)/2.

The information and procedures used and disclosed in Miyauchi S., et al., Cancer Res. 2020 Feb. 15; 80 (4): 732-746 are hereby incorporated by reference in their entirety. The information and procedures (e.g., protocols) disclosed will be implemented for the study of S-rimantadine, R-rimantadine, and/or racemic rimantadine.

### Example 8. In-Vitro Anti-Viral Methods

The direct anti-viral activity of enantiomers of rimantadine (e.g., S-rimantadine) will be tested against HPV viral replication using in-vitro HPV viral replication assays. S-rimantadine will demonstrate equivalent or increased direct HPV anti-viral activity compared to racemic rimantadine or R-rimantadine.

### Plasmid

Snls-Cre expression plasmid pCAGGS-nlsCre will be used. pNeo-loxP HPV-18 and pNeo-loxP HPV-18 E6*I plasmids will be used. For both plasmids, the 34-bp loxP sites will flank the linear HPV-18 sequence upstream of nucleotide 7474 and downstream from nucleotide 7473. The vector will carry the Neomycin resistance marker gene selectable in bacteria and in mammalian cells. In the HPV-18 E6*I mutant, the intron coding sequence (nucleotides 234-415) in the predominant E6*I mRNA will be deleted. For trans-complementation experiments, the empty vector-only retrovirus pLC and pLJ HPV-18 URR-E6 or URR-E6/E7 retro-viruses will be used. Each expresses the Neomycin resistance gene (Cheng et al. 1995. Differentiation-dependent up-regulation of the human papillomavirus E7 gene reactivates cellular DNA replication in suprabasal differentiated keratinocytes. Genes & Dev. 9:2335-2349; Chien et al. 2002. Alternative fates of keratinocytes transduced by human papillomavirus type 18 E7 during squamous differentiation. J. Virol. 76:2964-2972). All plasmids will be purified by banding (e.g., in CsCl-ethidium bromide equilibrium density gradients).

### HPV-18 Virion Recovery and Titer Determination

HPV-18 virions will be recovered from day-14 or day-16 epithelia as described (Favre, M. 1975. Structural polypeptides of rabbit, bovine, and human papillomaviruses. J. Virol. 15:1239-1247). To titer the virus, aliquots of the virus stocks will be digested with DNase I (Invitrogen), which will then be inactivated by heating for 5 min at 100 °C. Packaged viral DNA will then be purified by digestion with Proteinase K and phenol/chloroform extractions. Serial dilutions of viral DNA will be analyzed by real-time quantitative PCR using, for example, SYBR GreenER qPCR SuperMix (Invitrogen) and primers J and K, disclosed in Supplemental Table 1 of Wang HK. et al., Genes Dev. 2009 Jan. 15; 23 (2): 181-194. As standards, purified pNeo-LoxP HPV-18 plasmid DNA will be serially diluted to ~40 to 4×10⁸ copies per well. Forty cycle PCR amplification reactions in triplicate will be performed (e.g., in 384-well plates using the ABI 7900HT). Data will then be processed (e.g., with the use of SDS2.1 software (Applied Biosystems)).

### HPV-18 Infectivity Assays

Approximately 1×10⁵ primary human keratinocytes (PHKs) will be inoculated with various amounts of virus stock, corresponding to an MOI of 5200, 1040, 208, 42, 10, 2, 1, or 0 in 1 mL of K-SFM and incubated overnight. The medium will be changed and the cells will be cultured for four more days. Total RNA will then be extracted (e.g., with the use of Trizol (Invitrogen)). Reverse transcription will be conducted in a 50-mL reaction on 10 mg of RNA. One microliter of RT reaction will then then subjected to 30 cycles of PCR or nested PCR amplification (30 cycles each) in a 35-mL reaction mixture to generate a cDNA fragment of the spliced HPV-18 E6-E7-E1\hat{ }E4, RNA, or the b-actin mRNA, as described (Meyers et al. 2002. Infectious virions produced from a human papillomavirus type 18/16 genomic DNA chimera. J. Virol. 76:4723-4733). Fifteen micro-liters of each reaction will be resolved by electrophoresis in a 2% agarose gel and visualized by ethidium bromide staining. PHKs will also be infected with various MOIs in K-SFM overnight and developed into raft cultures, fixed on day 14, and processed as described.

The PHKs receiving varying amounts of virus stock will then be exposed to varying concentrations of R-rimantadine, S-rimantadine, and/or racemic rimantadine over a period of time (e.g., 1 day, 2 days, 3 days, 5 days, 7 days, and/or 10 days). The information and procedures (e.g., protocols) disclosed will be implemented for the study of S-rimantadine, R-rimantadine, and/or racemic rimantadine.

The information and procedures used and disclosed in Wang HK. et al., Genes Dev. 2009 Jan. 15; 23 (2): 181-194 are hereby incorporated by reference in their entirety. The information and procedures (e.g., protocols) disclosed will be implemented for the study of S-rimantadine, R-rimantadine, and/or racemic rimantadine.

### Example 9. In Vivo Central Nervous System (“Cns”) Assays

Studies will be conducted to determine the effects of R-rimantadine, S-rimantadine, and racemic rimantadine on the CNS of living animals (e.g., mice and/or rats). Varying doses of R-rimantadine, S-rimantadine, and racemic rimantadine will be studied and the following tests. Animals receiving S-rimantadine will demonstrate less CNS toxicity at similar doses of R-rimantadine and racemic rimantadine. Additionally, animals receiving S-rimantadine will be capable of receiving higher doses of the respective agent as compared to animals receiving R-rimantadine or racemic rimantadine before exhibiting signs and/or symptoms of CNS toxicity. Additionally, mice receiving S-rimantadine will better tolerate signs and symptoms of CNS toxicity as compared to mice receiving similar doses of R-rimantadine and racemic rimantadine.

### a) Rotarod

CNS toxicity associated with the use of R-rimantadine, S-rimantadine, and racemic rimantadine will be studied with the use of the rotarod system (e.g., Rotor Rod System, San Diego Instruments). Use of the Rotor Rod system will allow study of the CNS toxicity potentially caused by R-rimantadine, S-rimantadine, and racemic rimantadine by allowing observation of motor coordination in animals (e.g., mice or rats).

Animals will receive doses (e.g., varying doses) of R-rimantadine, S-rimantadine, or racemic rimantadine. After a period of time (e.g., 1 hour, 2 hours, 3 hours, 5 hours, 10 hours, 1 day, 2 days, 3 days, 5 days, 7 days, and/or 10 days) after receiving a dose, the potential CNS effects will be measured with the use of the rotarod system. Animals receiving S-rimantadine will demonstrate less adverse CNS effects and toxicity when compared to R-rimantadine and racemic rimantadine. In particular, animals (e.g., mice or rats) receiving S-rimantadine will demonstrate less abnormal motor coordination. The information and procedures used and disclosed in Rotor Rod, San Diego Instruments, available at https://sandiegoinstruments.com/product/rotor-rod/; ROTOR-ROD™ System, Biomedical and Obesity Research Core, College of Education and Human Sciences, University of Nebraska-Lincoln, available at https://cehs.unl.edu/borc/rotor-rod % E2%84% A2-system/; Castagne et al., CNS Safety Pharmacology, Reference Module in Biomedical Research, 2014; Dunham NW and Miya TS (1957) A note on a simple apparatus for detecting neurological deficit in rats and mice. Journal of the American Pharmaceutical Association, American Pharmaceutical Association (Baltimore) 46:208-209; Bohlen et al., Calibration of rotational acceleration for the rotarod test of rodent motor coordination, Journal of Neuroscience Methods (2009) 178:10-14; Shiotsuki et al., A rotarod test for evaluation of motor skill learning. J Neurosci Methods. 2010 Jun. 15; 189 (2): 180-5. doi: 10.1016/j.jneumeth.2010.03.026. Epub 2010 Mar. 30. PMID: 20359499; and Rustay N R, Wahlsten D, and Crabbe J C (2003) Influence of task parameters on rotarod performance and sensitivity to ethanol in mice. Behavioural Brain Research 141:237-249, are hereby incorporated by reference in their entirety. The information and procedures (e.g., protocols) disclosed will be implemented for the study of S-rimantadine, R-rimantadine, and/or racemic rimantadine.

### b) Photobeam Activity System-Home Cage

CNS toxicity associated with the use of R-rimantadine, S-rimantadine, and racemic rimantadine will be studied with the use of a Photobeam Activity System-Home Cage (San Diego Instruments). Use of the photobeam activity system-home cage will allow study of the animal's locomotive activity. Animals receiving R-rimantadine will demonstrate less CNS toxicity as evidenced by photobeam activity system-home cage testing.

Animals (e.g., mice or rats) will receive doses (e.g., varying doses) of R-rimantadine, S-rimantadine, or racemic rimantadine. After a period of time (e.g., 1 hour, 2 hours, 3 hours, 5 hours, 10 hours, 1 day, 2 days, 3 days, 5 days, 7 days, and/or 10 days) after receiving a dose, the potential CNS effects will be measured with the use of the photobeam activity system-home cage. Animals receiving S-rimantadine will demonstrate less adverse CNS effects and toxicity when compared to R-rimantadine and racemic rimantadine. In particular, animals (e.g., mice or rats) receiving S-rimantadine will demonstrate less abnormal locomotor activity. The information and procedures used and disclosed in Photobeam Activity System-Home Cage, San Diego Instruments, available at https://sandiegoinstruments.com/product/pas-homecage/, and Tatem et al., Behavioral and locomotor measurements using an open field activity monitoring system for skeletal muscle diseases. J Vis Exp. 2014 Sep. 29; (91): 51785. doi: 10.3791/51785. PMID: 25286313; PMCID: PMC4672952 are hereby incorporated by reference in its entirety. The information and protocols set forth in these disclosures will be used for the study of R-rimantadine, S-rimantadine, and racemic rimantadine. The study will be used and potentially modified to analyze aspects of the animal's physiological responses related to the CNS, such as circadian rhythm and anxiety.

### c) Irwin Test/Functional Observation Battery (FOB)

CNS toxicity associated with the use of R-rimantadine, S-rimantadine, and racemic rimantadine will be studied with the use of an Irwin Test and FOB. Use of the Irwin test and FOB will allow study of the qualitative effects of R-rimantadine, S-rimantadine, and racemic rimantadine. Animals receiving R-rimantadine will demonstrate less CNS toxicity as evidenced by the Irwin Test/FOB test.

Animals (e.g., mice or rats) will receive doses (e.g., varying doses (e.g., four different doses)) of R-rimantadine, S-rimantadine, or racemic rimantadine. After a period of time (e.g., 1 hour, 2 hours, 3 hours, 5 hours, 10 hours, 1 day, 2 days, 3 days, 5 days, 7 days, and/or 10 days) after receiving a dose, the behavior and physiological functions of the animals (e.g., mice or rats), will be studied. Animals receiving S-rimantadine will demonstrate less adverse CNS effects and toxicity when compared to R-rimantadine and racemic rimantadine. In particular, animals receiving S-rimantadine are will demonstrate less abnormal behavior and physiological function and similar doses, and animals receiving R-rimantadine will tolerate higher doses before demonstrating either observable effects on behavior and physiological function and/or higher doses before demonstrating clear behavioral toxicity. The information and procedures used and disclosed in Castagne et al., CNS Safety Pharmacology, Reference Module in Biomedical Research, 2014, Irwin S (1968), Comprehensive observational assessment: Ia. A systematic, quantitative procedure for assessing the behavioral and physiologic state of the mouse, Psychopharmacologia 13:222-257, Esteve J, Farre A J, and Roser R (1988) Pharmacological profile of droxicam, General Pharmacology 19:49-54, Mattson et al., (1996) A performance standard for clinical and functional observational battery examination of rats. Journal of the American College of Toxicology, 15:239-250, and Roux et al., Primary observation (Irwin) test in rodents for assessing acute toxicity of a test agent and its effects on behavior and physiological function. Curr. Protoc. Pharmacol. 2005 Jan. 1; Chapter 10: Unit 10.10. doi: 10.1002/0471141755.ph1010s27. PMID: 22294127, are hereby incorporated by reference in their entirety. The information and protocols set forth in these disclosures will be used for the study of R-rimantadine, S-rimantadine, and racemic rimantadine.

### d) Morris Water Maze Test

CNS toxicity associated with the use of R-rimantadine, S-rimantadine, and racemic rimantadine will be studied with the use of a Morris Water Maze Test. The Morris Water Maze Test will allow the study of potential CNS toxicity experienced by animal (e.g., mouse or rat) by testing the animal's spatial learning ability. Animals receiving R-rimantadine will demonstrate less CNS toxicity as evidenced by Morris Water Maze testing.

Animals (e.g., mice or rats) will receive doses (e.g., varying doses (e.g., four different doses)) of R-rimantadine, S-rimantadine, or racemic rimantadine. After a period of time (e.g., 1 hour, 2 hours, 3 hours, 5 hours, 10 hours, 1 day, 2 days, 3 days, 5 days, 7 days, and/or 10 days) after a dose, animals will be put into the maze. Animals receiving S-rimantadine will demonstrate less adverse CNS effects and toxicity when compared to R-rimantadine and racemic rimantadine. In particular, animals receiving S-rimantadine will demonstrate less inhibition of their spatial learning ability. The information and procedures used and disclosed in Vorhees et al., Morris water maze: procedures for assessing spatial and related forms of learning and memory, Nat Protoc 1, 848-858 (2006). https://doi.org/10.1038/nprot.2006.116, and Castagne et al., CNS Safety Pharmacology, Reference Module in Biomedical Research, 2014, Morris RGM (1981) Spatial localization does not require the presence of local cues, Learning and Motivation 12:239-260, are hereby incorporated by reference in their entirety. The information and protocols set forth in these disclosures will be used for the study of R-rimantadine, S-rimantadine, and racemic rimantadine.

### e) Electroencephalogram (EEG) Scans

CNS toxicity associated with the use of R-rimantadine, S-rimantadine, and racemic rimantadine will be studied with the use of EEG scans. EEG scans will allow the study of an animal's (e.g., mouse or rate) electrical activity in the brain. Animals receiving R-rimantadine will demonstrate less CNS toxicity as evidenced by EEG testing.

Animals (e.g., mice or rats) will receive doses (e.g., varying doses (e.g., four different doses)) of R-rimantadine, S-rimantadine, or racemic rimantadine. After a period of time (e.g., 1 hour, 2 hours, 3 hours, 5 hours, 10 hours, 1 day, 2 days, 3 days, 5 days, 7 days, and/or 10 days) after receiving a dose, EEG signals from the animals will be recorded. Animals receiving S-rimantadine will demonstrate less adverse CNS effects and toxicity when compared to R-rimantadine and racemic rimantadine. In particular, animals receiving S-rimantadine will demonstrate less abnormal EEG signals as compared to animals receiving to R-rimantadine and racemic rimantadine. The information and procedures used and disclosed in Vogler et al., Low Cost Electrod Assembly for EEg Recordings in Mice, Front. Neurosci., 14 Nov. 2017, https://doi.org/10.3389/fnins.2017.00629; Danhof M and Visser S A (2002) Pharmaco-electroencephalography and pharmacokinetic-pharmacodynamic modeling in drug development: focus on preclinical steps. Methods & Findings in Experimental & Clinical Pharmacology 24 ((Suppl D): 127-128; Itil™ and Itil KZ (1995) Quantitative EEG Brain Mapping In Psychotropic Drug Development, Drug Treatment Selection, and Monitoring. American Journal of Therapy 2:359-367; and Protocol for Rat Sleep EEG, NeuroDetective International, available at https://www.ndineuroscience.com/userfiles/Rat_Sleep_EEG_Methods.pdf, are hereby incorporated by reference in their entirety. The information and protocols set forth in these disclosures will be used for the study of R-rimantadine, S-rimantadine, and racemic rimantadine.

### Example 10. In Vitro Central Nervous System (“Cns”) Assays

Studies will be conducted to determine the effects of R-rimantadine, S-rimantadine, and racemic rimantadine on the changes in anatomy and/or physiology associated with CNS toxicity. Tissues obtained from animals (e.g., living or deceased) (e.g., mice and/or rats)) receiving S-rimantadine will demonstrate less changes as compared to baseline or normal (e.g., within acceptable limits) tissues when compared against tissues obtained from animals receiving R-rimantadine or racemic rimantadine.

Animals receiving S-rimantadine will demonstrate less physiological and/or anatomical changes due to CNS toxicity when compared to animals receiving similar doses of R-rimantadine and racemic rimantadine. Animals receiving S-will be capable of receiving higher doses of the S-rimantadine than animals receiving R-rimantadine and racemic rimantadine before exhibiting physiological and/or anatomical changes associated with CNS toxicity. Varying doses of R-rimantadine, S-rimantadine, and racemic rimantadine will be studied and with the use of at least the following tests. The information and protocols set forth in these disclosures will be used for the study of R-rimantadine, S-rimantadine, and racemic rimantadine.

### Brain Slice/Whole-Cell Patch-Clamp

CNS toxicity associated with the use of R-rimantadine, S-rimantadine, and racemic rimantadine will be studied with the use of Brain Slice/Whole-Cell Patch-Clamp studies. Brain Slice/Whole-Cell Patch-Clamp electrophysiology will allow for analysis of the biophysical mechanism (e.g., ionic currents) of neural computation and pathology in neuronal cells. Animals receiving R-rimantadine will demonstrate less CNS toxicity (e.g., less anatomical and/or physiological changes) as evidenced by brain slice/whole-cell patch-clamp tests.

Animals (e.g., mice or rats) will receive doses (e.g., varying doses (e.g., four different doses)) of R-rimantadine, S-rimantadine, or racemic rimantadine. After a period of time (e.g., 1 hour, 2 hours, 3 hours, 5 hours, 10 hours, 1 day, 2 days, 3 days, 5 days, 7 days, and/or 10 days) after receiving a dose, the animals will be euthanized and brain slices will be obtained and analyzed. Alternatively, whole-cell patch claim may be conducted in vivo. In such a case, after the period of time after receiving a dose, the animal will be analyzed without being euthanized. Animals receiving S-rimantadine will demonstrate less adverse CNS effects and toxicity when compared to animals receiving R-rimantadine or racemic rimantadine. In particular, animals receiving S-rimantadine will demonstrate less abnormal biophysical mechanisms of neural computation and pathology (e.g., ionic currents) than animals receiving either R-rimantadine or racemic rimantadine. The information and procedures used and disclosed in Kodandaramaiah et al., Automated whole-cell patch-clamp electrophysiology of neurons in vivo, Nat Methods. 2012 June; 9(6): 585-7. doi: 10.1038/nmeth. 1993. Epub 2012 May 6. PMID: 22561988; PMCID: PMC3427788, is hereby incorporated by reference in its entirety. The information and protocols set forth in these disclosures will be used for the study of R-rimantadine, S-rimantadine, and racemic rimantadine.

### Example 11: X-Ray Powder Diffraction of the Crystalline Type A Fumarate Salt of 2-S-Rimantidine

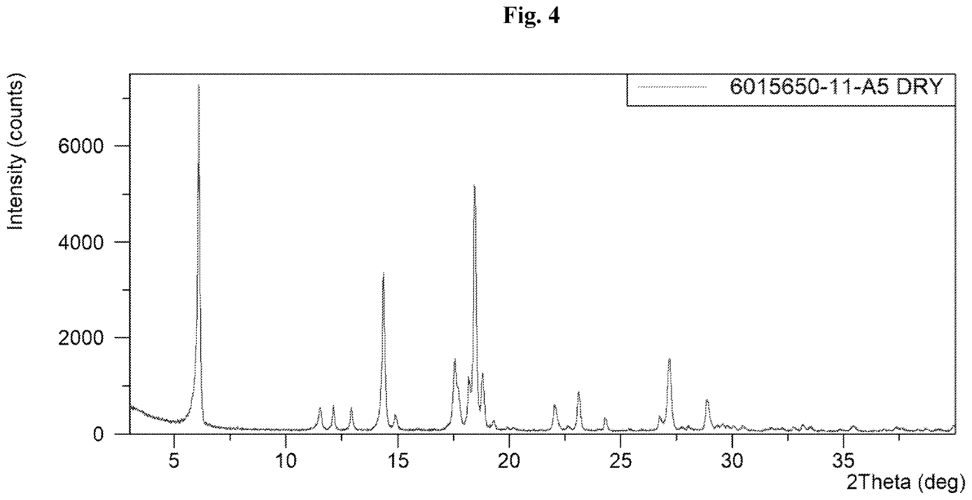

The crystalline type A fumarate salt of 2-S-Rimantidine was synthesized by methods described herein. The resulting crystalline sample was subject to X-ray powder diffraction for analysis. Table 4 shows the XRPD diffraction pattern observed when the crystalline Type A fumarate salt was analyzed. Additionally, FIG. 4 demonstrates the same data in diffractogram format.

TABLE 4

| | | | Relative |
|---|---|---|---|
| Pos. [°2Th.] | Height [cts] | d-spacing [Å] | Intensity [%] |
| 6.098394 | 6282.996000 | 14.49309 | 100.00 |
| 11.530040 | 434.340100 | 7.67492 | 6.91 |
| 12.137020 | 431.554100 | 7.29242 | 6.87 |
| 12.938090 | 443.979300 | 6.84265 | 7.07 |
| 14.365630 | 3192.905000 | 6.16574 | 50.82 |
| 14.919970 | 268.353900 | 5.93788 | 4.27 |
| 17.568310 | 1362.473000 | 5.04829 | 21.69 |
| 18.459990 | 5075.091000 | 4.80640 | 80.78 |
| 18.815160 | 1097.633000 | 4.71647 | 17.47 |
| 19.317420 | 132.631500 | 4.59496 | 2.11 |
| 19.886020 | 26.350830 | 4.46484 | 0.42 |
| 22.048430 | 514.263700 | 4.03159 | 8.19 |
| 22.661970 | 52.083530 | 3.92381 | 0.83 |
| 23.116720 | 793.254800 | 3.84764 | 12.63 |
| 24.299230 | 257.608200 | 3.66301 | 4.10 |
| 25.410320 | 29.414040 | 3.50531 | 0.47 |
| 25.999700 | 10.415790 | 3.42717 | 0.17 |
| 26.737080 | 255.656900 | 3.33430 | 4.07 |
| 27.176680 | 1459.346000 | 3.28136 | 23.23 |
| 27.766880 | 40.350610 | 3.21294 | 0.64 |
| 28.018830 | 56.761960 | 3.18462 | 0.90 |
| 28.851960 | 565.198100 | 3.09453 | 9.00 |
| 29.615770 | 50.827050 | 3.01644 | 0.81 |
| 30.042940 | 46.164570 | 2.97451 | 0.73 |
| 30.468260 | 73.641780 | 2.93395 | 1.17 |
| 31.647700 | 31.326230 | 2.82725 | 0.50 |
| 32.728050 | 67.101970 | 2.73635 | 1.07 |
| 33.171990 | 100.194600 | 2.70074 | 1.59 |
| 33.529790 | 74.051120 | 2.67273 | 1.18 |
| 34.049870 | 11.371280 | 2.63309 | 0.18 |
| 35.401370 | 82.708220 | 2.53561 | 1.32 |
| 37.449870 | 52.242390 | 2.40148 | 0.83 |
| 38.687800 | 33.108380 | 2.32745 | 0.53 |

### Example 12: X-Ray Powder Diffraction of the Crystalline Type A Tartrate Salt of 2-S-Rimantidine

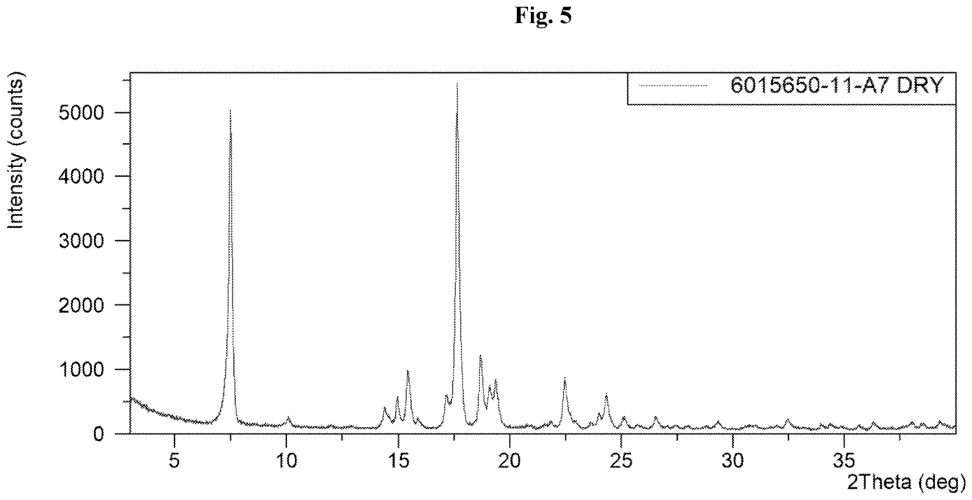

The crystalline Type A tartrate salt of 2-S-Rimantidine was synthesized by methods described herein. The resulting crystalline sample was subject to X-ray powder diffraction for analysis. Table 5 shows the XRPD diffraction pattern observed when the crystalline Type A tartrate salt was analyzed. Additionally, FIG. 5 demonstrates the same data in diffractogram format.

TABLE 5

| | | | Relative |
|---|---|---|---|
| Pos. [°2Th.] | Height [cts] | d-spacing [Å] | Intensity [%] |
| 7.500752 | 4715.048000 | 11.78628 | 89.83 |
| 10.104410 | 111.793100 | 8.75434 | 2.13 |
| 11.971530 | 34.719730 | 7.39285 | 0.66 |
| 12.881900 | 18.384860 | 6.87237 | 0.35 |
| 14.390700 | 283.136900 | 6.15505 | 5.39 |
| 14.962340 | 428.866700 | 5.92116 | 8.17 |
| 15.435140 | 822.433200 | 5.74083 | 15.67 |
| 15.923670 | 99.989520 | 5.56579 | 1.90 |
| 17.161790 | 468.955300 | 5.16694 | 8.93 |
| 17.649530 | 5249.105000 | 5.02524 | 100.00 |
| 18.682800 | 1039.974000 | 4.74958 | 19.81 |
| 19.092660 | 560.764200 | 4.64854 | 10.68 |
| 19.379290 | 711.242300 | 4.58042 | 13.55 |
| 20.896740 | 31.804290 | 4.25111 | 0.61 |
| 21.529510 | 42.948430 | 4.12758 | 0.82 |
| 21.848960 | 74.647790 | 4.06795 | 1.42 |
| 22.481110 | 742.757100 | 3.95497 | 14.15 |
| 23.640730 | 64.717700 | 3.76353 | 1.23 |
| 23.978380 | 186.073900 | 3.71130 | 3.54 |
| 24.327630 | 490.107600 | 3.65880 | 9.34 |
| 25.124300 | 144.945500 | 3.54456 | 2.76 |
| 25.711960 | 40.023390 | 3.46487 | 0.76 |
| 26.535340 | 168.953800 | 3.35919 | 3.22 |
| 27.474370 | 38.622270 | 3.24648 | 0.74 |
| 27.961400 | 31.583210 | 3.19103 | 0.60 |
| 28.804580 | 20.224110 | 3.09951 | 0.39 |
| 29.371890 | 84.335920 | 3.04092 | 1.61 |
| 31.969840 | 29.143360 | 2.79949 | 0.56 |
| 32.438450 | 111.068000 | 2.76012 | 2.12 |
| 33.983800 | 61.341840 | 2.63806 | 1.17 |
| 35.650280 | 55.794360 | 2.51847 | 1.06 |
| 36.298940 | 83.444670 | 2.47494 | 1.59 |
| 37.129010 | 12.288380 | 2.42150 | 0.23 |
| 38.078000 | 59.869450 | 2.36330 | 1.14 |
| 38.480980 | 42.180640 | 2.33948 | 0.80 |
| 39.260030 | 64.740260 | 2.29483 | 1.23 |

### Example 13: X-Ray Powder Diffraction of the Crystalline Type a Galactarate Salt of 2-S-Rimantidine

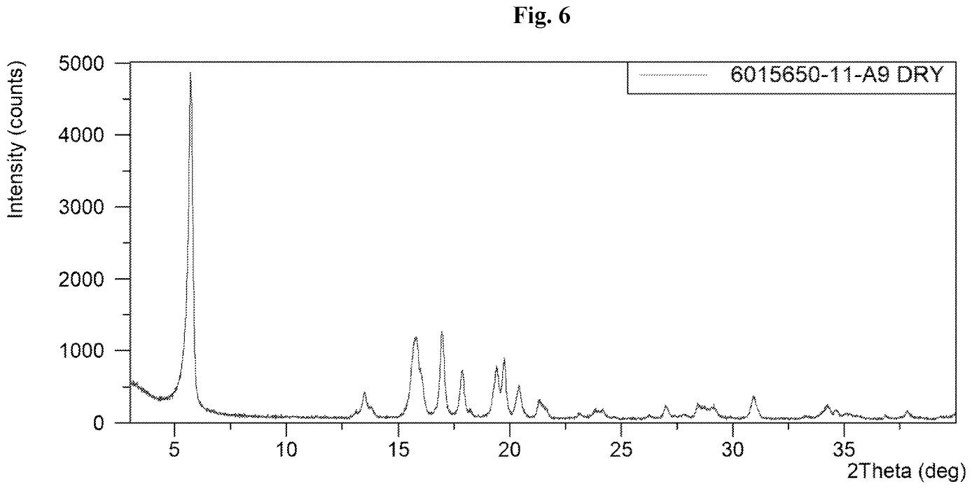

The crystalline Type A galactarate salt of 2-S-Rimantidine was synthesized by methods described herein. The resulting crystalline sample was subject to X-ray powder diffraction for analysis. Table 6 shows the XRPD diffraction pattern observed when the crystalline type A galactarate salt was analyzed. Additionally, FIG. 6 demonstrates the same data in diffractogram format.

TABLE 6

| | | | Relative |
|---|---|---|---|
| Pos. [°2Th.] | Height [cts] | d-spacing [Å] | Intensity [%] |
| 5.715397 | 4549.583000 | 15.46341 | 100.00 |
| 13.506820 | 333.720600 | 6.55578 | 7.34 |
| 15.857550 | 969.224300 | 5.58885 | 21.30 |
| 16.960960 | 1121.581000 | 5.22767 | 24.65 |
| 17.899260 | 591.546900 | 4.95569 | 13.00 |
| 19.433320 | 637.571200 | 4.56781 | 14.01 |
| 19.760180 | 754.831700 | 4.49299 | 16.59 |
| 20.429320 | 390.302300 | 4.34731 | 8.58 |
| 21.325020 | 221.577700 | 4.16669 | 4.87 |
| 23.133680 | 41.228080 | 3.84486 | 0.91 |
| 23.818840 | 78.084150 | 3.73579 | 1.72 |
| 26.301120 | 32.453140 | 3.38857 | 0.71 |
| 26.993780 | 147.393800 | 3.30318 | 3.24 |
| 27.838740 | 22.962460 | 3.20481 | 0.50 |
| 28.405050 | 137.762500 | 3.14219 | 3.03 |
| 29.181410 | 119.867600 | 3.06034 | 2.63 |
| 30.932300 | 285.131400 | 2.89099 | 6.27 |
| 33.297500 | 20.086610 | 2.69085 | 0.44 |
| 34.274250 | 129.335500 | 2.61636 | 2.84 |
| 36.860990 | 37.385440 | 2.43849 | 0.82 |
| 37.825650 | 84.841410 | 2.37849 | 1.86 |

### Example 14: X-Ray Powder Diffraction of the Crystalline Type B Galactarate Salt of 2-S-Rimantidine

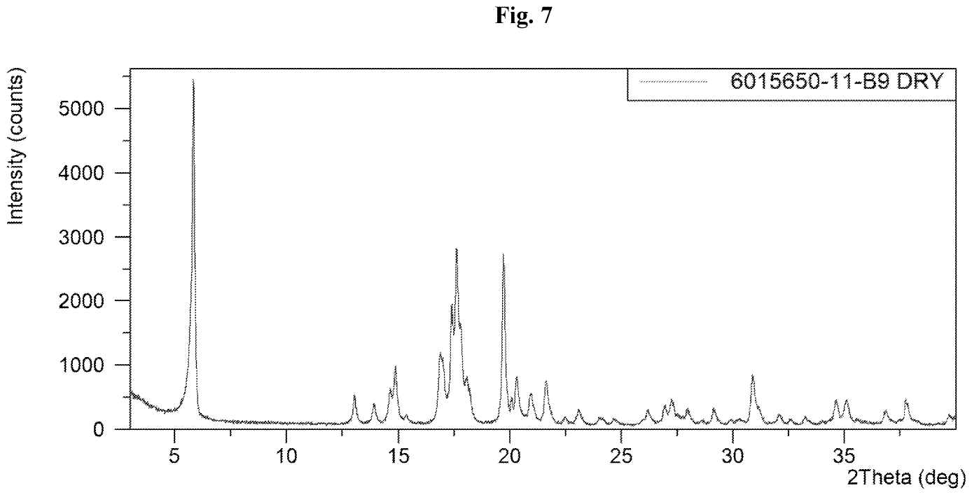

The crystalline Type B galactarate salt of 2-S-Rimantidine was synthesized by methods described herein. The resulting crystalline sample was subject to X-ray powder diffraction for analysis. Table 7 shows the XRPD diffraction pattern observed when the crystalline type B galactarate salt was analyzed. Additionally, FIG. 7 demonstrates the same data in diffractogram format.

TABLE 7

| | | | Relative |
|---|---|---|---|
| Pos. [°2Th.] | Height [cts] | d-spacing [Å] | Intensity [%] |
| 5.834619 | 5134.106000 | 15.14770 | 100.00 |
| 13.041820 | 422.334600 | 6.78846 | 8.23 |
| 13.918130 | 262.476600 | 6.36296 | 5.11 |
| 14.642410 | 482.753500 | 6.04981 | 9.40 |
| 14.893300 | 763.625700 | 5.94845 | 14.87 |
| 15.381230 | 114.292000 | 5.76083 | 2.23 |
| 16.870430 | 1027.347000 | 5.25551 | 20.01 |
| 17.622760 | 2665.918000 | 5.03281 | 51.93 |
| 18.136160 | 539.171800 | 4.89148 | 10.50 |
| 19.725200 | 2507.594000 | 4.50087 | 48.84 |
| 20.312130 | 632.889100 | 4.37213 | 12.33 |
| 20.931440 | 379.652000 | 4.24414 | 7.39 |
| 21.622340 | 597.688300 | 4.11007 | 11.64 |
| 22.489730 | 76.522670 | 3.95347 | 1.49 |
| 23.103330 | 203.933700 | 3.84984 | 3.97 |
| 24.005370 | 79.667050 | 3.70719 | 1.55 |
| 24.660840 | 70.561000 | 3.61012 | 1.37 |
| 26.178700 | 165.422700 | 3.40414 | 3.22 |
| 26.950270 | 219.118000 | 3.30841 | 4.27 |
| 27.244840 | 303.034100 | 3.27331 | 5.90 |
| 27.967100 | 180.961800 | 3.19039 | 3.52 |
| 28.604150 | 32.135640 | 3.12077 | 0.63 |
| 29.118880 | 210.872200 | 3.06677 | 4.11 |
| 29.898960 | 54.202620 | 2.98851 | 1.06 |
| 30.253720 | 45.618790 | 2.95427 | 0.89 |
| 30.870760 | 689.269700 | 2.89661 | 13.43 |
| 32.090500 | 126.768900 | 2.78924 | 2.47 |
| 32.601900 | 57.150120 | 2.74665 | 1.11 |
| 33.231380 | 98.809170 | 2.69605 | 1.92 |
| 34.609890 | 306.420800 | 2.59176 | 5.97 |
| 35.114090 | 314.042800 | 2.55569 | 6.12 |
| 35.590100 | 30.451060 | 2.52259 | 0.59 |
| 36.871600 | 174.865900 | 2.43781 | 3.41 |
| 37.745990 | 332.900100 | 2.38332 | 6.48 |
| 39.678890 | 83.951320 | 2.27157 | 1.64 |

### Example 15: X-Ray Powder Diffraction of the Crystalline Type A Benzoate Salt of 2-S-Rimantidine

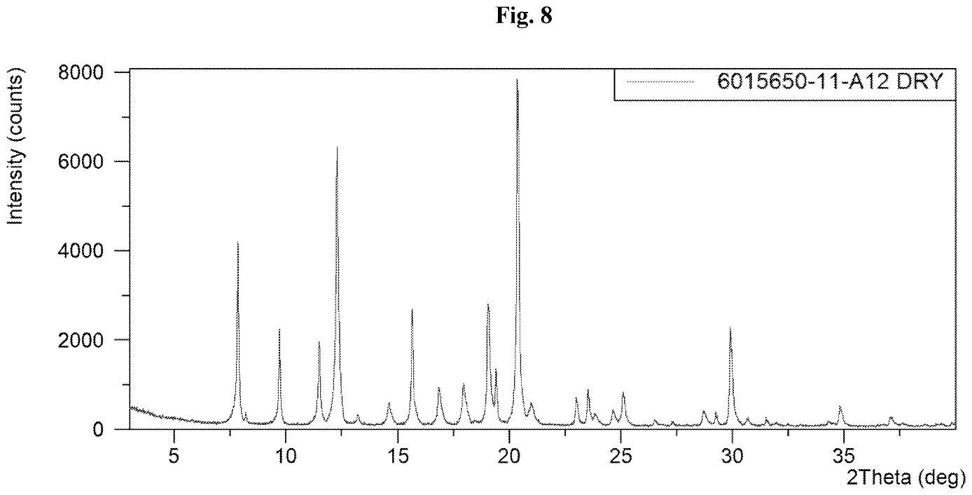

The crystalline Type A benzoate salt of 2-S-Rimantidine was synthesized by methods described herein. The resulting crystalline sample was subject to X-ray powder diffraction for analysis. Table 8 shows the XRPD diffraction pattern observed when the crystalline Type A benzoate salt was analyzed. Additionally, FIG. 8 demonstrates the same data in diffractogram format.

TABLE 8

| | | | Relative |
|---|---|---|---|
| Pos. [°2Th.] | Height [cts] | d-spacing [Å] | Intensity [%] |
| 7.852941 | 3433.361000 | 11.25847 | 47.45 |
| 8.218088 | 135.235800 | 10.75903 | 1.87 |
| 9.720021 | 1699.248000 | 9.09966 | 23.49 |
| 11.495610 | 1672.255000 | 7.69783 | 23.11 |
| 12.298320 | 6022.935000 | 7.19713 | 83.24 |
| 13.227150 | 182.560900 | 6.69376 | 2.52 |
| 14.608820 | 460.614900 | 6.06364 | 6.37 |
| 15.662350 | 2292.676000 | 5.65806 | 31.69 |
| 16.842700 | 773.202800 | 5.26410 | 10.69 |
| 17.949170 | 840.452200 | 4.94202 | 11.62 |
| 19.065890 | 2661.561000 | 4.65500 | 36.79 |
| 19.434000 | 934.324200 | 4.56765 | 12.91 |
| 20.382370 | 7235.354000 | 4.35722 | 100.00 |
| 20.989710 | 423.362300 | 4.23249 | 5.85 |
| 23.018920 | 537.346200 | 3.86377 | 7.43 |
| 23.534780 | 715.143600 | 3.78024 | 9.88 |
| 23.863080 | 222.859700 | 3.72897 | 3.08 |
| 24.639460 | 317.033300 | 3.61320 | 4.38 |
| 25.108660 | 664.581800 | 3.54674 | 9.19 |
| 26.541250 | 106.784200 | 3.35846 | 1.48 |
| 27.330590 | 69.250640 | 3.26323 | 0.96 |
| 28.709980 | 299.528100 | 3.10951 | 4.14 |
| 29.247310 | 253.109800 | 3.05359 | 3.50 |
| 29.912310 | 2155.655000 | 2.98720 | 29.79 |
| 30.667620 | 101.039400 | 2.91533 | 1.40 |
| 31.503720 | 131.164500 | 2.83984 | 1.81 |
| 34.290650 | 67.917000 | 2.61515 | 0.94 |
| 34.821090 | 384.582800 | 2.57652 | 5.32 |
| 37.098720 | 165.992200 | 2.42340 | 2.29 |
| 37.614560 | 39.496280 | 2.39135 | 0.55 |
| 38.641060 | 35.848500 | 2.33015 | 0.50 |
| 39.397340 | 28.900610 | 2.28715 | 0.40 |

### Example 16: X-Ray Powder Diffraction of the Crystalline Type A Benzenesulfonate Salt of 2-S-Rimantidine

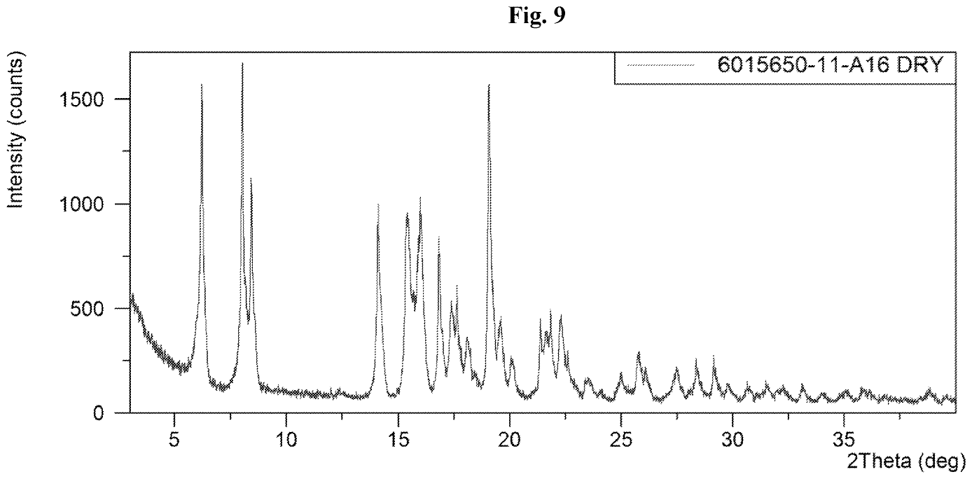

The crystalline benzenesulfonate salt of 2-S-Rimantidine was synthesized by methods described herein. The resulting crystalline sample was subject to X-ray powder diffraction for analysis. Table X shows the XRPD diffraction pattern observed when the crystalline Type A benzenesulfonate salt was analyzed. Additionally, FIG. 9 demonstrates the same data in diffractogram format.

TABLE 9

| | | | Relative |
|---|---|---|---|
| Pos. [°2Th.] | Height [cts] | d-spacing [Å] | Intensity [%] |
| 6.216367 | 1252.018000 | 14.21831 | 93.66 |
| 8.025520 | 1336.748000 | 11.01675 | 100.00 |
| 8.447595 | 843.597200 | 10.46723 | 63.11 |
| 14.119920 | 796.117100 | 6.27247 | 59.56 |
| 15.398860 | 826.688800 | 5.75428 | 61.84 |
| 16.012240 | 844.345500 | 5.53520 | 63.16 |
| 16.843030 | 633.982200 | 5.26400 | 47.43 |
| 17.353390 | 330.426500 | 5.11032 | 24.72 |
| 18.115630 | 224.311500 | 4.89698 | 16.78 |
| 19.081860 | 1295.366000 | 4.65114 | 96.90 |
| 19.638210 | 294.222100 | 4.52061 | 22.01 |
| 20.124240 | 139.686900 | 4.41252 | 10.45 |
| 21.384070 | 300.006900 | 4.15532 | 22.44 |
| 22.271890 | 335.889600 | 3.99165 | 25.13 |
| 23.440850 | 52.434730 | 3.79517 | 3.92 |
| 25.004000 | 103.617800 | 3.56134 | 7.75 |
| 25.739630 | 186.477800 | 3.46121 | 13.95 |
| 27.511690 | 134.510100 | 3.24216 | 10.06 |
| 28.388960 | 133.060700 | 3.14394 | 9.95 |
| 29.155170 | 145.656800 | 3.06303 | 10.90 |
| 29.791720 | 54.769070 | 2.99902 | 4.10 |
| 30.821950 | 30.303740 | 2.90109 | 2.27 |
| 31.539370 | 52.830390 | 2.83671 | 3.95 |
| 32.270400 | 45.119050 | 2.77410 | 3.38 |
| 33.167430 | 54.893690 | 2.70110 | 4.11 |
| 33.968690 | 24.294880 | 2.63920 | 1.82 |
| 35.128450 | 38.124390 | 2.55468 | 2.85 |
| 35.761520 | 36.580720 | 2.51089 | 2.74 |
| 38.832470 | 43.207560 | 2.31911 | 3.23 |
| 39.517230 | 14.623900 | 2.28049 | 1.09 |

### Example 17: X-Ray Powder Diffraction of the Crystalline Type B Benzenesulfonate Salt of 2-S-Rimantidine

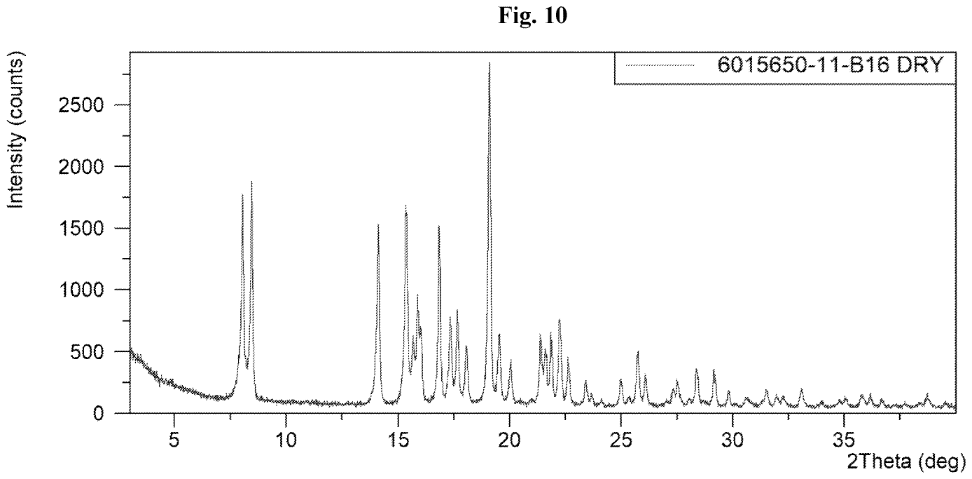

The crystalline Type B salt of 2-S-Rimantidine was synthesized by methods described herein. The resulting crystalline sample was subject to X-ray powder diffraction for analysis. Table 10 shows the XRPD diffraction pattern observed when the crystalline Type B benzenesulfonate salt was analyzed. Additionally, FIG. 10 demonstrates the same data in diffractogram format.

TABLE 10

| | | | Relative |
|---|---|---|---|
| Pos. [°2Th.] | Height [cts] | d-spacing [Å] | Intensity [%] |
| 8.044667 | 1436.713000 | 10.99057 | 54.21 |
| 8.453488 | 1565.351000 | 10.45995 | 59.07 |
| 14.139090 | 1357.615000 | 6.26401 | 51.23 |
| 15.373830 | 1573.853000 | 5.76359 | 59.39 |
| 15.931690 | 691.868500 | 5.56301 | 26.11 |
| 16.862220 | 1291.466000 | 5.25806 | 48.73 |
| 17.347400 | 628.045500 | 5.11208 | 23.70 |
| 17.678230 | 639.976100 | 5.01715 | 24.15 |
| 18.073330 | 414.128500 | 4.90835 | 15.63 |
| 19.108330 | 2650.177000 | 4.64476 | 100.00 |
| 19.541540 | 538.989500 | 4.54276 | 20.34 |
| 20.052510 | 296.282000 | 4.42814 | 11.18 |
| 21.392710 | 506.733400 | 4.15366 | 19.12 |
| 21.625820 | 405.685600 | 4.10942 | 15.31 |
| 21.870380 | 545.528100 | 4.06401 | 20.58 |
| 22.257080 | 661.085200 | 3.99427 | 24.94 |
| 22.648780 | 323.777300 | 3.92607 | 12.22 |
| 23.404940 | 178.392000 | 3.80091 | 6.73 |
| 23.689890 | 81.871800 | 3.75583 | 3.09 |
| 24.137730 | 41.689460 | 3.68716 | 1.57 |
| 24.993490 | 195.709300 | 3.56282 | 7.38 |
| 25.394210 | 43.873360 | 3.50750 | 1.66 |
| 25.741770 | 398.816600 | 3.46092 | 15.05 |
| 26.088590 | 218.161800 | 3.41570 | 8.23 |
| 27.299650 | 95.843500 | 3.26686 | 3.62 |
| 27.556210 | 162.570200 | 3.23702 | 6.13 |
| 28.376120 | 290.610700 | 3.14533 | 10.97 |
| 29.158980 | 269.575800 | 3.06264 | 10.17 |
| 29.824910 | 104.891200 | 2.99576 | 3.96 |
| 30.633200 | 56.263810 | 2.91853 | 2.12 |
| 31.541060 | 104.259600 | 2.83657 | 3.93 |
| 31.961360 | 73.437880 | 2.80022 | 2.77 |
| 33.073490 | 139.682600 | 2.70856 | 5.27 |
| 35.071560 | 59.661800 | 2.55869 | 2.25 |
| 35.794710 | 79.943050 | 2.50864 | 3.02 |
| 36.199550 | 68.861400 | 2.48151 | 2.60 |
| 36.707910 | 41.300770 | 2.44830 | 1.56 |
| 37.731800 | 21.669940 | 2.38419 | 0.82 |
| 38.721220 | 82.476750 | 2.32551 | 3.11 |
| 39.529390 | 33.657260 | 2.27981 | 1.27 |

### Other Embodiments

It is to be understood that while the invention has been described in conjunction with the detailed description thereof, the foregoing description is intended to illustrate and not limit the scope of the invention which is defined by the scope of the appended claims. Other aspects, advantages, and modification are within the scope of the following claims.

1. A salt comprising 2-S-Rimantidine and fumaric acid, tartaric acid, galactaric acid, benzoic acid, or benzenesulfonic acid salt.

2. The salt of claim 1, wherein the salt is anhydrous.

3. The salt of claim 1, wherein the salt is crystalline.

4. The salt of claim 3, wherein
(i) when the salt is, fumaric acid, the salt comprises an x-ray powder diffraction pattern comprising a peak at about 6.09 2θ;
(ii) when the salt is tartaric acid, the salt comprises an x-ray powder diffraction pattern comprising a peal at about 7.50 2θ;
(iii) when the salt is galactaric acid, the salt comprises an x-ray powder diffraction pattern comprising a peal at about 5.71 2θ;
(iv) when the salt is benzoic acid, the salt comprises an x-ray powder diffraction pattern comprising a peal at about 7.85 2θ; and
(v) when the salt is benzenesulfonic acid salt, the salt comprises an x-ray powder diffraction pattern comprising a peal at about 6.21 2θ.

5. The salt of claim 4, wherein when the salt is (i) the salt further comprises peaks at about 14.36 2θ, about 17.56 2θ, about 18.45 2θ, about 18.81 2θ, and/or about 27.17 2θ; when the salt is (ii) the salt further comprises peaks at about 17.64 2θ, about 18.68 2θ, about 15.43 2θ, about 19.37 2θ, and/or about 22.48 2θ; when the salt is (iii) the salt further comprises peaks at about 15.85 2θ, about 16.96 2θ, about 19.76 2θ, and/or about 19.43 2θ; when the salt is (iv) the salt further comprises peaks at about 9.72 2θ, about 11.49 2θ, about 12.29 2θ, about 15.66 2θ, about 19.96 2θ, about 29.38 2θ, and/or about 29.91 2θ; and when the salt is (v) the salt further comprises peaks at about 8.9229, about 8.4429, about 14.11 2θ, about 15.39 2θ, about 16.91 2θ, and/or about 19.98 2θ.

6-29. (canceled)

30. A crystalline form of 2-S-Rimantidine, wherein the XRPD peaks are about 14.36 2θ, about 17.56 2θ, about 18.45 2θ, about 18.81 2θ, and/or about 27.17 2θ.

31. A composition comprising a salt of 2-S rimantadine of claim 1.

32. The composition of claim 31, wherein the salt is a fumarate salt, tartrate salt, galactarate salt, benzoate salt, benzenesulfonate salt, or a combination thereof.

33. (canceled)

34. The composition of claim 32, wherein the fumarate salt is fumarate salt type A.

35. The composition of claim 34, characterized by one or more of:
a. a peak in an x-ray powder diffraction pattern (“XRPD”) diffractogram at about 6.99° 2θ;
b. a peak in an XRPD diffractogram at about 14.36° 2θ;
c. a peak in an XRPD diffractogram at about 17.56° 2θ;
d. a peak in an XRPD diffractogram at about 18.45° 2θ;
e. a peak in an XRPD diffractogram at about 18.81° 2θ; or
f. a peak in an XRPD diffractogram at about 27.17° 2θ.

36. (canceled)

37. The composition of claim 32, wherein the tartrate salt is tartrate salt type A.

38. The composition of claim 37, characterized by one or more of:
a. a peak in an x-ray powder diffraction pattern (“XRPD”) diffractogram at about 6.99° 2θ;
b. a peak in an XRPD diffractogram at about 7.59° 2θ;
c. a peak in an XRPD diffractogram at about 17.64° 2θ;
d. a peak in an XRPD diffractogram at about 18.68° 2θ;
e. a peak in an XRPD diffractogram at about 15.43° 2θ;
f. a peak in an XRPD diffractogram at about 19.37° 2θ; or
g. a peak in an XRPD diffractogram at about 22.48° 2θ.

39. (canceled)

40. The composition of claim 32, wherein the galactarate salt is a galactarate salt type A.

41. The composition of claim 40, characterized by one or more of:
a. a peak in an XRPD diffractogram at about 5.71° 2θ;
b. a peak in an XRPD diffractogram at about 15.85° 2θ;
c. a peak in an XRPD diffractogram at about 16.96° 2θ;
d. a peak in an XRPD diffractogram at about 19.76° 2θ; or
e. a peak in an XRPD diffractogram at about 19.43° 2θ.

42. The composition of claim 32, wherein the galactarate salt is a galactarate salt type B.

43. The composition of claim 42, characterized by one or more of:
a. a peak in an XRPD diffractogram at about 5.83° 2θ;
b. a peak in an XRPD diffractogram at about 14.89° 2θ;
c. a peak in an XRPD diffractogram at about 16.87° 2θ;
d. a peak in an XRPD diffractogram at about 17.62° 2θ;
e. a peak in an XRPD diffractogram at about 39.87° 2θ; or
f. a peak in an XRPD diffractogram at about 19.72° 2θ.

44. (canceled)

45. The composition of claim 32, wherein the benzoate salt is a benzoate salt type A.

46. The composition of claim 45, characterized by one or more of:
a. a peak in an XRPD diffractogram at about 7.85° 2θ;
b. a peak in an XRPD diffractogram at about 9.72° 2θ;
c. a peak in an XRPD diffractogram at about 11.49° 2θ;
d. a peak in an XRPD diffractogram at about 12.29° 2θ;
e. a peak in an XRPD diffractogram at about 15.66° 2θ;
f. a peak in an XRPD diffractogram at about 19.96° 2θ;
g. a peak in an XRPD diffractogram at about 29.38° 2θ; or
h. a peak in an XRPD diffractogram at about 29.91° 2θ.

47. (canceled)

48. The composition of claim 32, wherein the benzenesulfonate salt is a benzenesulfonate salt type A.

49. The composition of claim 48, characterized by one or more of:
a. a peak in an XRPD diffractogram at about 6.21° 2θ;
b. a peak in an XRPD diffractogram at about 8.92° 2θ;
c. a peak in an XRPD diffractogram at about 8.44° 2θ;
d. a peak in an XRPD diffractogram at about 14.11° 2θ;
e. a peak in an XRPD diffractogram at about 15.39° 2θ;
f. a peak in an XRPD diffractogram at about 16.91° 2θ; or
g. a peak in an XRPD diffractogram at about 19.98° 2θ.

50. The composition of claim 32, wherein the benzenesulfonate salt is a benzenesulfonate salt type B.

51. The composition of claim 31, characterized by one or more of:
a. a peak in an x-ray powder diffraction pattern (“XRPD”) diffractogram at about 8.04° 2θ;
b. a peak in an XRPD diffractogram at about 8.45° 2θ;
c. a peak in an XRPD diffractogram at about 14.13° 2θ;
d. a peak in an XRPD diffractogram at about 15.37° 2θ;
e. a peak in an XRPD diffractogram at about 16.86° 2θ; or
f. a peak in an XRPD diffractogram at about 19.10° 2θ.

52. The composition of claim 31, wherein the composition is crystalline.

53-107. (canceled)

108. The salt of 2-S-Rimantidine of claim 1, characterized by crystal particle size between 1-50 μm.

109-120. (canceled)

121. A method of treating cancer in a subject in need thereof, the method comprising administering to the subject a therapeutically effective amount of the salt of claim 1.

122. The method of claim 121, wherein the cancer is selected from one or more of melanoma, head and neck cancer, lung cancer, colon cancer, breast cancer, esophageal cancer, pancreatic cancer, prostate cancer, cervical cancer, and stomach cancer.

123. The method of claim 121, wherein the cancer is a sarcoma, carcinoma, lymphoma, or leukemia.

124-126. (canceled)

127. The method of claim 121, wherein the cancer is an HPV-associated cancer.

128. (canceled)

129. The method of claim 121, wherein one or more cancer cells from the subject express a human papilloma virus (HPV) protein.

130-133. (canceled)

134. A method of treating cancer in a subject, the method comprising:
a. detecting in a sample from the subject a cancer cell that expresses a human papilloma virus (HPV) protein; and
b. administering to the subject a therapeutically effective amount of the salt of claim 1.

135. (canceled)

136. The method of claim 134, wherein the HPV protein is one or more of an E5, E6, or E7 HPV protein.

137. (canceled)

138. The method of claim 134, wherein the cancer is selected from the group consisting of head and neck cancer, mucosal squamous cell carcinomas, cutaneous squamous cell carcinomas, hepatic cancer, cervical cancer, vaginal cancer, vulvar cancer, penile cancer, and anal cancer.

139. The method of claim 121, wherein the method further comprises administering an additional anti-cancer agent.

140. (canceled)

141. The method of claim 139, wherein the additional anti-cancer agent is an immunotherapy agent.

142. The method of claim 141, wherein the immunotherapy agent is an immune checkpoint inhibitor.

143. The method of claim 142, wherein the immune checkpoint inhibitor targets one or more of: CTLA-4, PD-1, PD-L1, BTLA, LAG-3, A2AR, TIM-3, B7-H3, VISTA, and IDO.

144. The method of claim 142, wherein the immune checkpoint inhibitor is selected form the group consisting of: ipilimumab, nivolumab, pembrolizumab, atezolizumab, avelumab, durvalumab, tremelimumab, cemiplimab, and a combination thereof.

145-146. (canceled)

147. A method of treating a precancerous HPV lesion in a subject needing treatment comprising administering a therapeutically effective amount of the salt of claim 1.

148. (canceled)

149. The method of claim 147, wherein the HPV precancerous lesion is a lesion of the cervix, skin, urethra, nasal cavity, paranasal sinus, larynx, tracheobronchial mucosa or oral cavity.

150. The method of claim 147, wherein the HPV precancerous lesion expresses one or more HPV proteins selected from one or more of E5, E6, or E7 HPV protein.

151-155. (canceled)

156. A composition comprising: a salt of claim 1, and one or more immune checkpoint inhibitors.

157. (canceled)

158. The salt of claim 1, wherein salt comprises enantiomerically pure 2-S-rimantadine.

159-160. (canceled)

161. A method of preparing a salt of 2-S-Rimantidine comprising
i. dissolving 2-S-Rimantidine in a first solvent to form a first solution;
ii. adding a second solvent to the solution to form a second solution;
iii. cooling the second solution; and
iv. evaporating the second solution.

162. The method of claim 161, wherein the first solvent is selected from a group consisting of methanol, ethanol, chloroform, and water.


SEQUENCE LISTING (20 sequences)

SEQ ID NO: 1 - DNA, 20 bp, synthetic construct
  source 1..20: mol_type = other DNA
gcacagggacataacaatgg

SEQ ID NO: 2 - DNA, 20 bp, synthetic construct
  source 1..20: mol_type = other DNA
gcgcagggccacaataatgg

SEQ ID NO: 3 - DNA, 20 bp, synthetic construct
  source 1..20: mol_type = other DNA
gcacagggacataataatgg

SEQ ID NO: 4 - DNA, 20 bp, synthetic construct
  source 1..20: mol_type = other DNA
gcccagggccacaacaatgg

SEQ ID NO: 5 - DNA, 20 bp, synthetic construct
  source 1..20: mol_type = other DNA
gctcagggtttaaacaatgg

SEQ ID NO: 6 - DNA, 20 bp, synthetic construct
  source 1..20: mol_type = other DNA
cgtcccaaaggaaactgatc

SEQ ID NO: 7 - DNA, 20 bp, synthetic construct
  source 1..20: mol_type = other DNA
cgacctaaaggaaactgatc

SEQ ID NO: 8 - DNA, 20 bp, synthetic construct
  source 1..20: mol_type = other DNA
cgtccaaaaggaaactgatc

SEQ ID NO: 9 - DNA, 18 bp, synthetic construct
  source 1..18: mol_type = other DNA
gccaaggggaaactgatc

SEQ ID NO: 10 - DNA, 20 bp, synthetic construct
  source 1..20: mol_type = other DNA
cgtcccaaaggatactgatc

SEQ ID NO: 11 - DNA, 20 bp, synthetic construct
  source 1..20: mol_type = other DNA
cgtccaaggggatactgatc

SEQ ID NO: 12 - DNA, 20 bp, synthetic construct
  source 1..20: mol_type = other DNA
cgacctaaagggaattgatc

SEQ ID NO: 13 - DNA, 20 bp, synthetic construct
  source 1..20: mol_type = other DNA
cgacctagtggaaattgatc

SEQ ID NO: 14 - DNA, 20 bp, synthetic construct
  source 1..20: mol_type = other DNA
cgaccaaggggatattgatc

SEQ ID NO: 15 - DNA, 18 bp, synthetic construct
  source 1..18: mol_type = other DNA
gcccaacggaaactgatc

SEQ ID NO: 16 - DNA, 20 bp, synthetic construct
  source 1..20: mol_type = other DNA
cgacccaagggaaactggtc

SEQ ID NO: 17 - DNA, 20 bp, synthetic construct
  source 1..20: mol_type = other DNA
cgtcctaaaggaaactggtc

SEQ ID NO: 18 - DNA, 20 bp, synthetic construct
  source 1..20: mol_type = other DNA
gcgacccaatgcaaattggt

SEQ ID NO: 19 - DNA, 20 bp, synthetic construct
  source 1..20: mol_type = other DNA
cgtccmarrggawactgatc

SEQ ID NO: 20 - DNA, 20 bp, synthetic construct
  source 1..20: mol_type = other DNA
gcmcagggwcataayaatgg